(19) United States
(12) Patent Application Publication
(10) Pub. No.: US 2009/0156540 A1
(43) Pub. Date: Jun. 18, 2009

(54) METHOD FOR DIAGNOSING AND/OR PREDICTING THE DEVELOPMENT OF AN ALLERGIC DISORDER AND AGENTS FOR TREATING AND/OR PREVENTING SAME

(76) Inventors: Katherine McKenna, Carine (AU); Patrick Holt, Nedlands (AU); Anthony Bosco, Coogee (AU); Peter Sly, East Perth (AU)

Correspondence Address: STITES & HARBISON PLLC, 1199 NORTH FAIRFAX STREET, SUITE 900, ALEXANDRIA, VA, 22314, US

(21) Appl. No.: 12/281,519

(22) PCT Filed: Mar. 7, 2007
(86) PCT No.: PCT/AU2007/000287

§ 371 Date: Dec. 19, 2008

(30) Foreign Application Priority Data
Mar. 7, 2006 (AU) 2006901142
Mar. 7, 2006 (AU) 2006901143

(51) Int. Cl. A61K 31/7088; C12Q 1/68; C07H 21/00; A61P 37/08

(52) U.S. Cl. 514/44; 435/6; 536/23.1

(57) ABSTRACT

The present invention relates to genes whose level of expression is different in allergic animals compared with non-allergic animals. In particular, the present invention relates to a method for predicting the development of an allergic disorder in a mammal by determining the gene expression pattern of a panel of specific sequences comprising CAMK2D and CDH1 within a nucleic acid pool that have been predetermined to either increase or decrease in response to allergy.

### FIELD

The present invention relates to genes whose level of expression is different in allergic animals compared with non-allergic animals. Thus, the present invention also relates to methods of diagnosis and/or prediction of allergic disorders. It also provides agents capable of treating or preventing allergic disorders, methods of monitoring the progress of therapy and/or methods of determining the potential responsiveness of individual mammals to particular forms of therapy. The present invention further relates to methods for treating or preventing allergy and methods of screening for agents capable of treating or preventing allergic disorders.

### BACKGROUND

Allergic disorders such as asthma, atopic dermatitis, hyper-IgE syndrome, Omenn's syndrome, and allergic rhinitis represent some of the most common and well characterised immune disorders in humans. Allergic disorders affect roughly 20 percent of all individuals in the United States. However, while there are a number of clinical test procedures for assessing allergies, the methods available for early diagnosis of allergy or for predicting whether an individual will develop allergy or determining which subtype of allergy an individual-patient has are imprecise and subject to high levels of patient-to-patient variability. The underlying reason for this variability is that allergic disorders are multifactorial in origin and involve the operation within different patients of different combinations of inflammatory mechanisms driven by the products of a large number of different genes. However the currently available tests measure the products of a very restricted range of genes. In other words, current immunological tests for allergy only provide superficial information about an individual's current immunological status.

Current treatment of allergic disorders includes allergen avoidance, pharmaceutical-based therapy and immunotherapy. Completely avoiding allergen exposure is the most logical approach, but this is very difficult or impossible to achieve in the vast majority of cases. Pharmaceuticals such as anti-histamines, steroids, beta-agonists and adrenaline are useful, but they only alleviate the symptoms of allergy without influencing its cause. In addition, pharmaceutical treatment is usually limited by undesirable side effects, particularly in the case of steroids.

Current immunotherapeutic approaches include desensitisation, allergen alteration aimed at reducing recognition by specific antibodies, and the use of allergen-derived peptides, which interfere in the cognate interaction between specific B and T cells. Desensitisation therapy involves repeated injections with increasing dosages of a crude allergen extract of the offending allergen. Although treatment with allergen extracts has been proven reasonably effective in the clinic for alleviating allergen-related symptoms, and is a common therapy used widely in allergy clinics today, the mechanism of desensitisation remains unclear. Furthermore, desensitisation therapy must be undertaken with extreme caution, as anaphylactic side effects may be significant or even fatal.

Accordingly, there is a need for more precise non-invasive methods for diagnosing and/or predicting the development of an allergic disorder in a mammal such as a human.

Furthermore, there is still a need in the art for more effective treatments for allergic disorders. Unfortunately, the development of these treatments has been hampered by the lack of understanding about the aetiology of allergy, which has still to be elucidated.

The inventors believe that they have developed a greater understanding of the mechanisms underlying allergy, which has enabled them to develop a more effective method of therapy and method of screening for agents capable of preventing and/or treating allergic disorders in mammals such as humans.

### SUMMARY

Accordingly, in a first aspect, the present invention provides a method for predicting the development of an allergic disorder in a mammal comprising the steps of: (a) contacting a cell of the mammal with an allergen; (b) contacting a cell of a non-allergic mammal with the same allergen used in step (a); (c) obtaining a sample of nucleic acid isolated from the cells in steps (a) and (b), wherein the nucleic acid is RNA or a cDNA copy of RNA; (d) determine the gene expression pattern of a panel of specific sequences comprising CAMK2D and CDH1 within each nucleic acid pool described in (c) that have been predetermined to either increase or decrease in response to allergy, where the gene expression pattern comprises the relative level of mRNA or cDNA abundance for the panel of specific sequences; and (e) compare the expression patterns in step (d), wherein the difference in the levels of expression is predictive of whether the mammal in step (a) will develop allergy.

In a second aspect, the present invention provides a method for diagnosing an allergic disorder in a mammal comprising the steps of: (a) contacting a cell of the mammal with an allergen; (b) contacting a cell of a non-allergic mammal with the same allergen used in step (a); (c) obtaining a sample of nucleic acid isolated from the cells in steps (a) and (b), wherein the nucleic acid is RNA or a cDNA copy of RNA; (d) determine the gene expression pattern of a panel of specific sequences comprising CAMK2D and CDH1 within each nucleic acid pool described in (c) have been predetermined to either increase or decrease in response to allergy, where the gene expression pattern comprises the relative level of mRNA or cDNA abundance for the panel of specific sequences; and (e) compare the expression patterns in step (d), wherein the difference in the levels of expression is diagnostic that the mammal in step (a) is allergic.

In a third aspect, the present invention provides a method for preventing or treating an allergic disorder in a mammal comprising the steps of:
(a) obtaining a pool of nucleic acid molecules isolated from the mammal's organ, tissue or cell, wherein the nucleic acid is RNA or a cDNA copy of RNA;
(b) determining the gene expression pattern of a panel of specific sequences within the pool of nucleic acid molecules described in (a) that have been predetermined to either increase or decrease in response to allergy, where the gene expression pattern comprises the relative level of mRNA or cDNA abundance for the panel of specific sequences and wherein said panel includes CAMK2D and CDH1;
(c) identify a gene expression pattern for one or more of the panel of specific sequences which is different when compared with the predetermined level of expression; and
(d) administering an agent capable of bringing the gene expression pattern to the predetermined level of expression.

In a fourth aspect the invention provides a method of selecting an agent for the treatment of a mammal having an allergic disorder, comprising:
(a) contacting a cell of an allergic mammal with a test agent;
(b) contacting a cell of a non-allergic mammal with the same test agent used in step (a);
(c) obtaining a sample of nucleic acid isolated from the cells in steps (a) and (b), wherein the nucleic acid is RNA or a cDNA copy of RNA;
(d) determine the gene expression pattern of a panel of specific sequences comprising CAMK2D and CDH1 within each nucleic acid pool described in (c) that have been predetermined to either increase or decrease in response to allergy, where the gene expression pattern comprises the relative level of mRNA or cDNA abundance for the panel of specific sequences; and
(e) compare the expression patterns in step (d), and if the levels of expression of said panel are similar then the test agent is useful in the treatment of a mammal with an allergy.

In a fifth aspect the invention provides a method of selecting a prophylactic agent for a mammal in which an allergic disorder is to be prevented, comprising:
(a) contacting a cell of suspected allergic mammal with a test agent;
(b) contacting a cell of a non-allergic mammal with the same test agent used in step (a);
(c) obtaining a sample of nucleic acid isolated from the cells in steps (a) and (b), wherein the nucleic acid is RNA or a cDNA copy of RNA;
(d) determine the gene expression pattern of a panel of specific sequences comprising CAMK2D and CDH1 within each nucleic acid pool described in (c) that have been predetermined to either increase or decrease in response to allergy, where the gene expression pattern comprises the relative level of mRNA or cDNA abundance for the panel of specific sequences; and
(e) compare the expression patterns in step (d), and if the levels of expression of said panel are similar then the test agent is useful as a prophylactic agent in the prevention of an allergy in the mammal.

In a sixth aspect the invention provides a method of screening for an agent capable of modulating the expression of a gene associated with an allergic disorder:
(a) contacting a cell of an allergic mammal with a test agent;
(b) contacting a cell of a non-allergic mammal with the same test agent used in step (a);
(c) obtaining a sample of nucleic acid isolated from the cells in steps (a) and (b), wherein the nucleic acid is RNA or a cDNA copy of RNA;
(d) determine the gene expression pattern of a panel of specific sequences comprising CAMK2D and CDH1 within each nucleic acid pool described in (c) that have been predetermined to either increase or decrease in response to allergy, where the gene expression pattern comprises the relative level of mRNA or cDNA abundance for the panel of specific sequences; and
(e) compare the expression patterns in step (d), and if the levels of expression of said panel are different in the presence of the test agent this indicates that the agent is capable of modulating the expression of CAMK2D and CDH1.

In a seventh aspect the invention provides a method of monitoring a mammal during therapy for an allergic disorder, comprising:
(a) contacting a cell of the mammal before therapy with an allergen;
(b) contacting a cell of the mammal under therapy with the same allergen used in step (a);
(c) contacting a cell of a non-allergic mammal with the same allergen used in step (a);
(d) obtaining a sample of nucleic acid isolated from the cells in steps (a), (b) and (c), wherein the nucleic acid is RNA or a cDNA copy of RNA;
(e) determine the gene expression pattern of a panel of specific sequences comprising CAMK2D and CDH1 within each nucleic acid pool described in (d) that have been predetermined to either increase or decrease in response to allergy, where the gene expression pattern comprises the relative level of mRNA or cDNA abundance for the panel of specific sequences; and
(f) compare the expression patterns in step (e) and determine whether the level of expression has changed during therapy,
   wherein a change in the level of expression during therapy is an indication of the progress of the therapy.

In an eighth aspect the invention provides a method of determining the potential responsiveness of an animal suffering from an allergic disorder to treatment for the allergic disorder, comprising:
(a) contacting a cell of an allergic mammal with an allergen;
(b) contacting a cell of a non-allergic mammal with the same allergen used in step (a);
(c) obtaining a sample of nucleic acid isolated from the cells in steps (a) and (b), wherein the nucleic acid is RNA or a cDNA copy of RNA;
(d) determine the gene expression pattern of a panel of specific sequences comprising CAMK2D and CDH1 within each nucleic acid pool described in (c) that have been predetermined to either increase or decrease in response to allergy, where the gene expression pattern comprises the relative level of mRNA or cDNA abundance for the panel of specific sequences; and
(e) compare the expression patterns in step (d), wherein a difference in the levels of expression is indicative of the potential responsiveness of the animal to the therapy.

In a ninth aspect the invention provides a method of predicting the risk of an animal suffering from an allergic disorder progressing to a more severe and/or persistent form of the allergic disorder, comprising:
(a) contacting a cell of an allergic mammal with an allergen;
(b) contacting a cell of a non-allergic mammal with the same allergen used in step (a);
(c) obtaining a sample of nucleic acid isolated from the cells in steps (a) and (b), wherein the nucleic acid is RNA or a cDNA copy of RNA;
(d) determine the gene expression pattern of a panel of specific sequences comprising CAMK2D and CDH1 within each nucleic acid pool described in (c) that have been predetermined to either increase or decrease in response to allergy, where the gene expression pattern comprises the relative level of mRNA or cDNA abundance for the panel of specific sequences; and
(e) compare the expression patterns in step (d), wherein any difference in the level of expression between the allergic mammal and non-allergic mammal is predictive of the risk of the allergic mammal developing a more severe and/or persistent form of the allergic disorder.

In a tenth aspect the invention provides a method of determining the immunological phenotype of an allergic disorder in an animal, comprising:
(a) contacting a cell of an allergic mammal with an allergen;
(b) contacting a cell of a non-allergic mammal with the same allergen used in step (a);
(c) obtaining a sample of nucleic acid isolated from the cells in steps (a) and (b), wherein the nucleic acid is RNA or a cDNA copy of RNA;
(d) determine the gene expression pattern of a panel of specific sequences comprising CAMK2D and CDH1 within each nucleic acid pool described in (c) that have been predetermined to either increase or decrease in response to allergy, where the gene expression pattern comprises the relative level of mRNA or cDNA abundance for the panel of specific sequences; and
(e) compare the expression patterns in step (d), wherein the level of expression is indicative of the immunological phenotype of the animal.

In some embodiments, the panel of specific sequences in any one of the first to tenth aspects, further comprises or consists of any one or more of SLC37A3, PALM2-AKAP2, NSMCE1, TSPAN13, SYTL3, SFRS8, FIP1L1, MAML3, TRIM4, SIAH1, ITPR1, ITSN2, CLCF1, CRLF1, CLIC5, IGJ, NFKBIZ, DLC1, GBP5, PEG10, HOMER2, ZBTB8, MOBKL2C, EDG3, MELK, PHC3, TTC3, KLK1, KCNV2, IL1F9, GBP1, SEL1, IL1R2, IFI44L or LIX1L.

In an eleventh aspect the invention provides an isolated molecule comprising one or more of:

a) the sequence of a nucleic acid selected from the group consisting of CAMK2D, CDH1, SLC37A3, PALM2-AKAP2, NSMCE1, TSPAN13, SYTL3, SFRS8, FIP1L1, MAML3, TRIM4, SIAH1, ITPR1, ITSN2, CLCF1, CRLF1, CLIC5, IGJ, NFKBIZ, DLC1, GBP5, PEG10, HOMER2, ZBTB8, MOBKL2C, EDG3, MELK, PHC3, TTC3, KLK1, KCNV2, IL1F9, GBP1, SEL1, IL1R2, IFI44L and LIX1L, or a biologically active fragment thereof;
b) an isolated nucleic acid molecule which is the complement of a sequence of a);
c) an isolated nucleic molecule which hybridises under stringent conditions to a nucleic acid molecule of a) or b); and/or
d) an isolated polypeptide encoded by a nucleic acid molecule of a), b) or c),
for use in the treatment or prevention of an allergic disorder.

In a twelfth aspect the invention provides a therapeutic or prophylactic agent, comprising one or more of:

a) an isolated nucleic acid molecule having the sequence of a nucleic acid selected from the group consisting of CAMK2D, CDH1, SLC37A3, PALM2-AKAP2, NSMCE1, TSPAN13, SYTL3, SFRS8, FIP1L1, MAML3, TRIM4, SIAH1, ITPR1, ITSN2, CLCF1, CRLF1, CLIC5, IGJ, NFKBIZ, DLC1, GBP5, PEG10, HOMER2, ZBTB8, MOBKL2C, EDG3, MELK, PHC3, TTC3, KLK1, KCNV2, IL1F9, GBP1, SEL1, IL1R2, IFI44L and LIX1L, or a biologically active fragment thereof;
b) an isolated nucleic acid molecule which is the complement of a sequence of a);
c) an isolated nucleic molecule which hybridises under stringent conditions to a nucleic acid molecule of a) or b); and/or
d) an isolated polypeptide encoded by a nucleic acid molecule of a), b) or c),
together with a pharmaceutically acceptable carrier.

The agent is for use in the treatment or prevention of an allergic disorder. The carrier may be selected from one or more of the group consisting of sterile water, sodium phosphate, mannitol, sorbitol, sodium chloride.

In a thirteenth aspect the invention provides a method of treating or preventing an allergic disorder, comprising the step of administering to a mammal one or more of:

a) an isolated nucleic acid molecule having the sequence of a gene selected from the group consisting of CAMK2D, CDH1, SLC37A3, PALM2-AKAP2, NSMCE1, TSPAN13, SYTL3, SFRS8, FIP1L1, MAML3, TRIM4, SIAH1, ITPR1, ITSN2, CLCF1, CRLF1, CLIC5, IGJ, NFKBIZ, DLC1, GBP5, PEG10, HOMER2, ZBTB8, MOBKL2C, EDG3, MELK, PHC3, TTC3, KLK1, KCNV2, IL1F9, GBP1, SEL1, IL1R2, IFI44L and LIX1L, or a biologically active fragment thereof;
b) an isolated nucleic acid molecule which is the complement of a sequence of a);
c) an isolated nucleic molecule which hybridises under stringent conditions to a nucleic acid molecule of a) or b);
d) an isolated polypeptide encoded by a nucleic acid molecule of a), b) or c); and/or
e) an agent capable of modulating the expression of a molecule of a), b), and/or c), or which specifically binds a polypeptide of d).

The agent may be a nucleic acid molecule which is antisense to the nucleic acid sequence of a gene selected from the group consisting of CAMK2D, CDH1, SLC37A3, PALM2-AKAP2, NSMCE1, TSPAN13, SYTL3, SFRS8, FIP1L1, MAML3, TRIM4, SIAH1, ITPR1, ITSN2, CLCF1, CRLF1, CLIC5, IGJ, NFKBIZ, DLC1, GBP5, PEG10, HOMER2, ZBTB8, MOBKL2C, EDG3, MELK, PHC3, TTC3, KLK1, KCNV2, IL1F9, GBP1, SEL1, IL1R2, IFI44L and LIX1L, or a biologically active fragment thereof. The agent may be a nucleic acid molecule which is antisense to the nucleic acid sequence of a gene selected from the group consisting of CAMK2D, SLC37A3, PALM2-AKAP2, NSMCE1, SFRS8, FIP1L1, MAML3, TRIM4, SIAH1, ITPR1, ITSN2, CLCF1, CRLF1, CLIC5, IGJ, NFKBIZ, DLC1, GBP5, PEG10, HOMER2, ZBTB8, MOBKL2C, EDG3, MELK, PHC3, TTC3, TSPAN13, and SYTL3, or a biologically active fragment thereof. Alternatively the agent that may specifically bind to a polypeptide of d) is a polyclonal or monoclonal antibody, or a biologically active fragment thereof.

In a fourteenth aspect the invention provides a kit for screening for an agent capable of treating or preventing an allergic disorder, comprising one or more of:

a) an isolated nucleic acid molecule having the sequence of a gene selected from the group consisting of CAMK2D, CDH1, SLC37A3, PALM2-AKAP2, NSMCE1, TSPAN13, SYTL3, SFRS8, FIP1L1, MAML3, TRIM4; SIAH1, ITPR1, ITSN2, CLCF1, CRLF1, CLIC5, IGJ, NFKBIZ, DLC1, GBP5, PEG10, HOMER2, ZBTB8, MOBKL2C, EDG3, MELK, PHC3, TTC3, KLK1, KCNV2, IL1F9, GBP1, SEL1, IL1R2, IFI44L and LIX1L, or a biologically active fragment thereof;
b) an isolated nucleic acid molecule which is the complement of a sequence of a);
c) an isolated nucleic molecule which hybridises under stringent conditions to a nucleic acid molecule of a) or b);
d) an isolated polypeptide encoded by a nucleic acid molecule of a), b) or c).

In a fifteenth aspect the invention provides a method of screening for an agent capable of treating or preventing an allergic disorder, comprising:

a) providing a panel of specific sequences comprising CAMK2D, CDH1, SLC37A3 and PALM2-AKAP2, or a biologically active fragment thereof, under conditions which allow expression of the specific sequences;
b) determining the level of expression of the specific sequences;
c) contacting the specific sequences with the agent;
d) determining whether the level of expression changes, wherein a change in the level of expression indicates that the agent is capable of treating or preventing an allergic disease.

In a sixteenth aspect the invention provides a microarray, comprising two or more allergy-associated genes selected from the group consisting of CAMK2D, CDH1, SLC37A3, PALM2-AKAP2, NSMCE1, TSPAN13, SYTL3, SFRS8, FIP1L1, MAML3, TRIM4, SIAH1, ITPR1, ITSN2, CLCF1, CRLF1, CLIC5, IGJ, NFKBIZ, DLC1, GBP5, PEG10, HOMER2, ZBTB8, MOBKL2C, EDG3, MELK, PHC3, TTC3, KLK1, KCNV2, IL1F9, GBP1, SEL1, IL1R2, IFI44L, and LIX1L, or a biologically active fragment thereof.

In a seventeenth aspect the invention provides a microfluidic device comprising two or more allergy-associated genes selected from the group consisting of CAMK2D, CDH1, SLC37A3, PALM2-AKAP2, NSMCE1, TSPAN13, SYTL3, SFRS8, FIP1L1, MAML3, TRIM4, SIAH1, ITPR1, ITSN2, CLCF1, CRLF1, CLIC5, IGJ, NFKBIZ, DLC1, GBP5, PEG10, HOMER2, ZBTB8, MOBKL2C, EDG3, MELK, PHC3, and TTC3, or a biologically active fragment thereof.

### BRIEF DESCRIPTION OF THE FIGURES

FIG. 1 shows the level of IL-4 mRNA expression in CD4+ cells after culture for 16 hrs. The level of expression is expressed as delta values (difference between unstimulated and HDM-stimulated cultures) for non-allergic (N) and allergic (A) individuals.

FIG. 2 shows the level of IL-4 mRNA expression in CD4+ cells after culture for 24 hrs. The level of expression is expressed as delta values (difference between unstimulated and HDM-stimulated cultures) for non-allergic (N) and allergic (A) individuals.

FIG. 3 shows the level of CAMK2D mRNA expression in CD4+ cells after culture for 24 hrs. The level of expression is expressed as delta values (difference between unstimulated and HDM-stimulated cultures) for non-allergic (N) and allergic (A) individuals.

FIG. 4 shows the level of CAMK2D mRNA expression in CD8+ cells after culture for 24 hrs. The level of expression is expressed as delta values (difference between unstimulated and HDM-stimulated cultures) for non-allergic (N) and allergic (A) individuals.

FIG. 5 shows the level of CAMK2D mRNA expression in CD4+ cells after culture for 24 hrs. The level of expression is expressed as delta values (difference between unstimulated and HDM-stimulated cultures) for non-allergic (N) and allergic (A) individuals.

FIG. 6 shows the level of CAMK2D mRNA expression in CD8+ cells after culture for 24 hrs. The level of expression is expressed as delta values (difference between unstimulated and HDM-stimulated cultures) for non-allergic (N) and allergic (A) individuals.

FIG. 7 shows the level of NSMCE1 mRNA expression in CD4+ cells after culture for 24 hrs. The level of expression is expressed as delta values (difference between unstimulated and HDM-stimulated cultures) for non-allergic (N) and allergic (A) individuals.

FIG. 8 shows the level of NSMCE1 mRNA expression in CD4+ cells after culture for 24 hrs. The level of expression is expressed as delta values (difference between unstimulated and HDM-stimulated cultures) for non-allergic (N) and allergic (A) individuals.

FIG. 9 shows the level of NSMCE1 mRNA expression in CD8+ cells after culture for 24 hrs. The level of expression is expressed as delta values (difference between unstimulated and HDM-stimulated cultures) for non-allergic (N) and allergic (A) individuals.

FIG. 10 shows the level of TSPAN13 mRNA expression in CD4+ cells after culture for 24 hrs. The level of expression is expressed as delta values (difference between unstimulated and HDM-stimulated cultures) for non-allergic (N) and allergic (A) individuals.

FIG. 11 shows the level of STYL3 mRNA expression in CD4+ cells after culture for 24 hrs. The level of expression is expressed as delta values (difference between unstimulated and HDM-stimulated cultures) for non-allergic (N) and allergic (A) individuals.

FIG. 12 shows the level of STYL3 mRNA expression in CD4+ cells after culture for 24 hrs. The level of expression is expressed as delta values (difference between unstimulated and HDM-stimulated cultures) for non-allergic (N) and allergic (A) individuals.

FIG. 13 shows the level of STYL3 mRNA expression in CD8+ cells after culture for 24 hrs. The level of expression is expressed as delta values (difference between unstimulated and HDM-stimulated cultures) for non-allergic (N) and allergic (A) individuals.

FIG. 14 shows a comparison of the level of CAMK2D mRNA expression 24 hours following HDM stimulation in purified CD4 T cells in an independent cohort of atopic. (n=10) and nonatopic (n=10) individuals as assessed by quantitative real-time PCR.

FIG. 15 shows a comparison of the level of CAMK2D mRNA expression 24 hours following HDM stimulation in purified CD4 T cells in an additional independent cohort of atopic (n=10) and nonatopic (n=10) individuals as assessed by quantitative real-time PCR.

FIG. 16 shows a comparison of the level of NSMCE1 mRNA expression 24 hours following HDM stimulation in purified CD4 T cells in an independent cohort of atopic (n=10) and nonatopic (n=10) individuals as assessed by quantitative real-time PCR.

FIG. 17 shows a comparison of the level of NSMCE1 mRNA expression 24 hours following HDM stimulation in purified CD4 T cells in an additional independent cohort of atopic (n=10) and nonatopic (n=10) individuals as assessed by quantitative real-time PCR.

FIG. 18 shows a comparison of the level of SYTL3 mRNA expression 24 hours following HDM stimulation in purified CD4 T cells in an independent cohort of atopic (n=10) and nonatopic (n=10) individuals as assessed by quantitative real-time PCR.

FIG. 19 shows a comparison of the level of SYTL3 mRNA expression 24 hours following HDM stimulation in purified CD4 T cells in an additional independent cohort of atopic (n=10) and nonatopic (n=10) individuals as assessed by quantitative real-time PCR.

FIG. 20 shows a comparison of the level of SLC37A3 mRNA expression 24 hours following HDM stimulation in purified CD4 T cells in an independent cohort of atopic (n=10) and nonatopic (n=10) individuals as assessed by quantitative real-time PCR.

FIG. 21 shows a comparison of the level of NFKBIZ mRNA expression 24 hours following HDM stimulation in purified CD4 T cells in an independent cohort of atopic (n=10) and nonatopic (n=10) individuals as assessed by quantitative real-time PCR.

FIG. 22A shows the coexpression network comprising the 16 functional modules, where the tree-like dendrogram connects genes together that have high interconnectivity (correlated expression levels), revealing separate branch-like structures of highly connected genes or network modules.

FIG. 22B shows a subset of the network shown in FIG. 22A in expanded form. Closer inspection of the coexpression network revealed that the principal genes mediating Th2-driven allergic inflammation (IL-4, IL-4R, IL-5, IL-9, IL-13) formed a “Th2 effector” module with 104 other genes.

### DETAILED DESCRIPTION OF THE PREFERRED EMBODIMENTS

Before describing the present invention in detail, it is to be understood that this invention is not limited to particularly exemplified methods of diagnosis and may, of course, vary. It is also to be understood that the terminology used herein is for the purpose of describing particular embodiments of the invention only, and is not intended to be limiting which will be limited only by the appended claims.

All publications, patents and patent applications cited herein, whether supra or infra, are hereby incorporated by reference in their entirety. However, publications mentioned herein are cited for the purpose of describing and disclosing the protocols and reagents which are reported in the publications and which might be used in connection with the invention. Nothing herein is to be construed as an admission that the invention is not entitled to antedate such disclosure by virtue of prior invention.

Furthermore, the practice of the present invention employs, unless otherwise indicated, conventional immunological techniques, chemistry and pharmacology within the skill of the art. Such techniques are well known to the skilled worker, and are explained fully in the literature. See, e.g., Coligan, Dunn, Ploegh, Speicher and Wingfield “Current protocols in Protein Science” (1999) Volume I and II (John Wiley & Sons Inc.); and Bailey, J. E. and Ollis, D. F., Biochemical Engineering Fundamentals, McGraw-Hill Book Company, NY, 1986.

It must be noted that as used herein and in the appended claims, the singular forms “a,” “an,” and “the” include plural reference unless the context clearly dictates otherwise. Thus, for example, a reference to “a gene” includes a plurality of such genes, and a reference to “an allergy” is a reference to one or more allergies, and so forth.

Unless defined otherwise, all technical and scientific terms used herein have the same meanings as commonly understood by one of ordinary skill in the art to which this invention belongs. Although any materials and methods similar or equivalent to those described herein can be used to practice or test the present invention, the preferred materials and methods are now described.

Without wishing to be bound by any particular theory or hypothesis, the inventors have observed and demonstrated that expression of one or more genes in allergen-stimulated cells, such as in peripheral blood mononuclear cells (PMBC) or T cells, occurs in mammals that are susceptible, pre-disposed or have an allergic disorder at a different level than in mammals that do not have the allergic disorder. For example, the inventors have noted that the level of expression from genes including CAMK2D, CDH1, SLC37A3, PALM2-AKAP2, NSMCE1, TSPAN13, SYTL3, SFRS8, FIP1L1, MAML3, TRIM4, SIAH1, ITPR1, ITSN2, CLCF1, CRLF1, CLIC5, IGJ, NFKBIZ, DLC1, GBP5, PEG10, HOMER2, ZBTB8, MOBKL2C, EDG3, MELK, PHC3, and TTC3, or combinations thereof (“genes of interest”), are higher in house dust mite (HDM)-stimulated PMBC or T cells from humans allergic to house dust mite than in subjects not allergic. In contrast, other genes may be actively down-regulated in HDM-stimulated PBMC from non-atopic individuals (normal individuals) but not down-regulated in corresponding PBMC samples from atopic (“allergic”) individuals. These genes are still considered indicative of the non-atopic phenotype, they are also considered to be representative of “protective” genes i.e. the product of these genes might in someway provide protection from the development of allergy. This observation can be used to distinguish allergic mammals from non-allergic, or less allergic mammals and thus has numerous applications, such as use diagnosis, prognosis, as well as methods of treating or preventing an allergic disorder in a mammal or selecting an agent for the treatment or prevention of an allergic disorder in a mammal.

By “propensity,” “pre-disposition” or “susceptibility” what is meant is that the level of expression of CAMK2D, CDH1, SLC37A3, PALM2-AKAP2, NSMCE1, TSPAN13, SYTL3, SFRS8, FIP1L1, MAML3, TRIM4, SIAH1, ITPR1, ITSN2, CLCF1, CRLF1, CLIC5, IGJ, NFKBIZ, DLC1, GBP5, PEG10, HOMER2, ZBTB8, MOBKL2C, EDG3, MELK, PHC3, and TTC3, or combinations thereof, are hereby “associated” with allergic disorders such that mammals that are pre-disposed or susceptible to allergic disorders have different amounts of the products of these genes than the amount of the products found in a “normal” or non-atopic mammal.

An “allergic disorder” or “allergic condition” refers to an abnormal biological function characterized by either an increased responsiveness of the trachea and bronchi to various stimuli or a disorder involving inflammation. The symptoms associated with these allergic disorders include, but are not limited to, cold, cold-like, and/or flu symptoms, cough, dermal irritation, dyspnea, lacrimation, rhinorrhea, sneezing and wheezing. Allergic disorders are also often associated with an increase in Th2 cytokines such as IL-4, IL-4R, IL-5, IL-9 and IL-13. Examples of allergic disorders include, but are not limited to, actinic dermatitis (or photodermatitis), allergic granulomatosis, allergic vasculitis, seborrheic dermatitis, symptomatic dermographism dermatitis, asthma, atopic dermatitis, bronchoconstriction, chronic airway inflammation, cosmetic dermatitis, Crohn's disease, dermatitis aestivalis, eczema, edema, eosinophilic gastroenteritis, eosinophilic granuloma, eosinophilic myocardial disease, eosinophilic chlorecystitis, episodic angioedema with eosinophilia, familial histiocytosis, food allergy, Grave's disease, hay fever, hypereosinophilic syndromes, hypersensitivity, hypertension, hyper-IgE syndrome, idiopathic pulmonary fibrosis, inflammatory bowel disease, mast cell degranulation, Omenn's syndrome, psoriasis, rhinitis, serum sickness, solar urticaria, ulcerative colitis and urticaria.

As used herein, the terms “allergic” or “atopic” refers to a mammal which has an allergic reaction generally caused by allergens such as, e.g., food, dander, or insect venom. Conversely a “non-allergic” or “non-atopic” mammal is one which does not have an allergic disorder caused by the allergen which causes the allergic disorder in the allergic mammal, or does not have an allergic disorder caused by any allergen.

As used herein, the term “mammal” or “mammalian” includes, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs. The terms do not denote a particular age, and thus both adult and immature individuals are intended to be covered. The methods described herein are intended for use in any of the above mammalian species, since the immune systems of all of these mammals operate similarly.

Thus, in some embodiments, the present invention encompasses a method for predicting the development of an allergic disorder in any mammal, including a human, as well as those mammals of economic and/or social importance to humans, including carnivores such as cats, dogs and larger felids and canids, swine such as pigs, hogs, and wild boars, ruminants such as cattle, oxen, sheep, giraffes, deer, goats, bison, and camels, and horses, and non-human primates such as apes and monkeys. Thus the invention encompasses the screening of livestock, including, but not limited to, domesticated swine, ruminants, horses, and the like, and zoo or endangered animals.

Primarily, the present invention is based on determining the level of expression of one or more nucleic acids or genes in a cell of a mammal. A “cell” may be any cell capable of being stimulated by an allergen, for example a peripheral blood mononuclear cell (PBMC) such as a T cell. PBMCs are cells present in the bloodstream and having one nucleus such as lymphocytes, macrophages, and monocytes. Lymphocytes are also present in lymph and lymph tissue. T cells are one type of lymphocyte and these cells can be further divided according to whether the CD4 or CD8 receptor is expressed on the surface of the cell.

The cell may be located in or isolated from any biological sample of a mammal. Accordingly, the term “biological sample” as used herein includes any biological material isolated from a mammal. Preferably, the biological sample is tissue or fluid isolated from bone marrow, plasma, serum, spinal fluid, lymph fluid, the external sections of the skin, respiratory, intestinal, and genitourinary tracts, tears, saliva, milk, whole blood, blood cells, tumours, organs, or in vivo cell culture constituents. More preferably, the biological sample is blood, lymph fluid or a blood component. Most preferably, the biological sample comprises bone marrow derived mononuclear cells from peripheral blood.

The biological sample may be tested using the techniques described herein directly after isolation or alternatively further processed in order to increase the quality of the data produced. In this regard, the inventors have noted from the literature that the selective expansion of allergen specific cells by initial stimulation with allergen is useful to induce proliferation and generates a “cell line” in which the frequency of the relevant cells are log scale greater than the same cells in a biological sample directly isolated from a mammal. The literature has also shown that, if required, the cells can be further concentrated and purified by cloning the specific cells.

In some embodiments, a biological sample such as peripheral blood is taken from a mammal that is suspected of, or susceptible to the development of an allergic disorder. The biological sample is then treated so as to substantially isolate leukocytes from the blood i.e. separate the leukocytes from (or otherwise substantially free from) other contaminant cells.

As used herein the term “isolated” means that a molecule of interest eg leukocyte is identified and separated and/or recovered from a component of its natural environment. Contaminant components of its natural environment are materials that would typically interfere with the use of the molecule.

Once isolated the biological sample is then exposed to an allergen such that a cell of the sample is contacted with the allergen. An “allergen” is an antigen which causes a hypersensitivity reaction in a mammal. Common allergens include pollen, house dust, animal dander, and various foods. The term “environmental allergen” as used herein refers to allergens that are specifically associated with the development of allergic disorders. For example, allergens might include those of animals, including the mite (e.g., Dermatophagoides pteronyssinus, Dermatophagoides farinae, Blomia tropicalis), such as the allergens der p1 (Scobie et al. (1994) Biochem. Soc. Trans. 22: 448S; Yssel et al. (1992) J. Immunol. 148: 738-745), der p2 (Chua et al. (1996) Clin. Exp. Allergy 26: 829-837), der p3 (Smith and Thomas (1996) Clin. Exp. Allergy 26: 571-579), der p5, der p V (Lin et al. (1994) J. Allergy Clin. Immunol. 94: 989-996), der p6 (Bennett and Thomas (1996) Clin. Exp. Allergy 26: 1150-1154), der p 7 (Shen et al. (1995) Clin. Exp. Allergy 25: 416-422), der f2 (Yuuki et al. (1997) Int. Arch. Allergy Immunol. 112: 44-48), der f3 (Nishiyama et al. (1995) FEBS Lett. 377: 62-66), der f7 (Shen et al. (1995) Clin. Exp. Allergy 25: 1000-1006); Mag 3 (Fujikawa et al. (1996) Mol. Immunol. 33: 311-319). Also of interest as allergens are the house dust mite allergens Tyr p2 (Eriksson et al. (1998) Eur. J. Biochem. 251: 443-447), Lep d1 (Schmidt et al. (1995) FEBS Lett. 370: 11-14), and glutathione S-transferase (O'Neill et al. (1995) Immunol Lett. 48: 103-107); the 25,589 Da, 219 amino acid polypeptide with homology with glutathione S-transferases (O'Neill et al. (1994) Biochim. Biophys. Acta. 1219: 521-528); Blo t 5 (Arruda et al. (1995) Int. Arch. Allergy Immunol. 107: 456-457); bee venom phospholipase A2 (Carballido et al. (1994) J. Allergy Clin. Immunol. 93: 758-767; Jutel et al. (1995) J. Immunol. 154: 4187-4194); bovine dermal/dander antigens BDA 11 (Rautiainen et al. (1995) J. Invest. Dermatol. 105: 660-663) and BDA20 (Mantyjarvi et al. (1996) J. Allergy Clin. Immunol. 97: 1297-1303); the major horse allergen Equ c1 (Gregoire et al. (1996) J. Biol. Chem. 271: 32951-32959); Jumper ant M. pilosula allergen Myr p I and its homologous allergenic polypeptides Myr p2 (Donovan et al. (1996) Biochem. Mol. Biol. Int. 39: 877-885); 1-13, 14, 16 kD allergens of the mite Blomia tropicalis (Caraballo et al. (1996) J. Allergy Clin. Immunol. 98: 573-579); the cockroach allergens Bla g Bd90K (Helm et al. (1996) J. Allergy Clin. Immunol. 98: 172-80) and Bla g 2 (Arruda et al. (1995) J. Biol. Chem. 270: 19563-19568); the cockroach Cr-PI allergens (Wu et al. (1996) J. Biol. Chem. 271: 17937-17943); fire ant venom allergen, Sol i 2 (Schmidt et al. (1996) J. Allergy Clin. Immunol. 98: 82-88); the insect Chironomus thummi major allergen Chi t 1-9 (Kipp et al. (1996) Int. Arch. Allergy Immunol. 110: 348-353); dog allergen Can f 1 or cat allergen Fel d 1 (Ingram et al. (1995) J. Allergy Clin. Immunol. 96: 449-456); albumin, derived, for example, from horse, dog or cat (Goubran Botros et al. (1996) Immunology 88: 340-347); deer allergens with the molecular mass of 22 kD, 25 kD or 60 kD (Spitzauer et al. (1997) Clin. Exp. Allergy 27: 196-200); and the 20 kd major allergen of cow (Ylonen et al. (1994) J. Allergy Clin. Immunol. 93: 851-858).

Pollen and grass allergens include, for example, Hor v9 (Astwood and Hill (1996) Gene 182: 53-62, Lig v1 (Batanero et al. (1996) Clin. Exp. Allergy 26: 1401-1410); Lol p 1 (Muller et al. (1996) Int. Arch. Allergy Immunol. 109: 352-355), Lol p II (Tamborini et al. (1995) Mol. Immunol. 32: 505-513), Lol pVA, Lol pVB (Ong et al. (1995) Mol. Immunol. 32: 295-302), Lol p 9 (Blaher et al. (1996) J. Allergy Clin. Immunol. 98: 124-132); Par J I (Costa et al. (1994) FEBS Lett. 341: 182-186; Sallusto et al. (1996) J. Allergy Clin. Immunol. 97: 627-637), Par j 2.0101 (Duro et al. (1996) FEBS Lett. 399: 295-298); Bet v1 (Faber et al. (1996) J. Biol. Chem. 271: 19243-19250), Bet v2 (Rihs et al. (1994) Int. Arch. Allergy Immunol. 105: 190-194); Dac g3 (Guerin-Marchand et al. (1996) Mol. Immunol. 33: 797-806); Phi p 1 (Petersen et al. (1995) J. Allergy Clin. Immunol. 95: 987-994), Phl p 5 (Muller et al. (1996) Int. Arch. Allergy Immunol. 109: 352-355), Phl p 6 (Petersen et al. (1995) Int. Arch. Allergy Immunol. 108: 55-59); Cry j I (Sone et al. (1994) Biochem. Biophys. Res. Commun. 199: 619-625), Cry j II (Namba et al. (1994) FEBS Lett. 353: 124-128); Cor a 1 (Schenk et al. (1994) Eur. J. Biochem. 224: 717-722); cyn d1 (Smith et al. (1996) J. Allergy Clin. Immunol. 98: 331-343), cyn d7 (Suphioglu et al. (1997) FEBS Lett. 402: 167-172); Pha a 1 and isoforms of Pha a 5 (Suphioglu and Singh (1995) Clin. Exp. Allergy 25: 853-865); Cha o 1 (Suzuki et al. (1996) Mol. Immunol. 33: 451-460); profilin derived, e.g, from timothy grass or birch pollen (Valenta et al. (1994) Biochem. Biophys. Res. Commun. 199: 106-118); P0149 (Wu et al. (1996) Plant Mol. Biol. 32: 1037-1042); Ory s1 (Xu et al. (1995) Gene 164: 255-259); and Amb a V and Amb t 5 (Kim et al. (1996) Mol. Immunol. 33: 873-880; Zhu et al. (1995) J. Immunol. 155: 5064-5073).

Fungal allergens include, but are not limited to, Cla h III of Cladosporium herbarum (Zhang et al. (1995) J. Immunol. 154: 710-717); Psi c 2, a fungal cyclophilin, from the basidiomycete Psilocybe cubensis (Homer et al. (1995) Int. Arch. Allergy Immunol. 107: 298-300); hsp 70 cloned from a cDNA library of Cladosporium herbarum (Zhang et al. (1996) Clin Exp Allergy 26: 88-95); the 68 kD allergen of Penicillium notatum (Shen et al. (1995) Clin. Exp. Allergy 26: 350-356); aldehyde dehydrogenase (ALDH) (Achatz et al. (1995) Mol. Immunol. 32: 213-227); enolase (Achatz et al. (1995) Mol. Immunol. 32: 213-227); YCP4 (Id.); acidic ribosomal protein P2 (Id.).

Suitable food allergens include, for example, profilin (Rihs et al. (1994) Int. Arch. Allergy Immunol. 105: 190-194); rice allergenic cDNAs belonging to the alpha-amylase/trypsin inhibitor gene family (Alvarez et al. (1995) Biochim Biophys Acta 1251: 201-204); the main olive allergen, Ole e I (Lombardero et al. (1994) Clin Exp Allergy 24: 765-770); Sin a 1, the major allergen from mustard (Gonzalez De La Pena et al. (1996) Eur J Biochem. 237: 827-832); parvalbumin, the major allergen of salmon (Lindstrom et al. (1996) Scand. J Immunol. 44: 335-344); apple allergens, such as the major allergen Mal d 1 (Vanek-Krebitz et al. (1995) Biochem. Biophys. Res. Commun. 214: 538-551); and peanut allergens, such as Ara h I (Burks et al. (1995) J Clin. Invest. 96: 1715-1721).

As used herein the term “contacting” includes both direct and indirect contacting. This step potentially constitutes the stimulation phase of the described method whereby the level of expression of one or more nucleic acids or genes of interest is modulated. A cell may be contacted with an allergen by any method known in the art, for example by adding the allergen to the fluid surrounding the cell in an amount sufficient to activate a gene of the cell. Alternatively, the cell may be added to a solution containing a suitable amount of an allergen. A suitable amount of allergen may be 1 μg/ml to 100 μg/ml. In some embodiments the amount of allergen may be 10 μg/ml to 100 μg/ml. In other embodiments to amount of allergen may be 30 μg/ml.

When the cell has been contacted with the allergen, a nucleic acid or gene of interest in the cell may be activated. As used herein the term “gene” means a length of DNA which encodes a particular protein or RNA molecule and may or may not include the 5′ and 3′ untranslated regions of the DNA. The terms “genes of the invention”, “genes of interest” and “allergy-associated genes” refer to genes which are shown to be associated with an allergic disorder in that an animal exhibiting clinical symptoms of an allergic disorder has a gene which is activated in the presence of an allergen at a different level to that of a non-allergic animal. Genes particularly suitable for use in the invention are CAMK2D, CDH1, SLC37A3, PALM2-AKAP2, NSMCE1, TSPAN13, SYTL3, SFRS8, FIP1L1, MAML3, TRIM4, SIAH1, ITPR1, ITSN2, CLCF1, CRLF1, CLIC5, IGJ, NFKBIZ, DLC1, GBP5, PEG10, HOMER2, ZBTB8, MOBKL2C, EDG3, MELK, PHC3, TTC3, KLK1, KCNV2, IL1F9, GBP1, SEL1, IL1R2, IFI44L, and LIX1L. Details of each of these genes are summarised in Table 1.

Nucleotide sequences of the invention may include sequences that differ by one or more nucleotide substitutions, additions or deletions, such as allelic variants, and will also include sequences that differ due to the degeneracy of the genetic code.

TABLE 1

| | Entrez | | | |
|---|---|---|---|---|
| Gene | ID | Source | Gene Name | Swiss Prot Function |
| CAMK2D | 817 | Early | calcium/calmodulin-dependent | CaM-kinase II (CAMK2) is a prominent kinase in the |
| | | cluster | protein kinase (CaM kinase) II | central nervous system that may function in long- |
| | | | delta | term potentiation and neurotransmitter release. |
| CDH1 | 999 | Early | cadherin 1, type 1, E-cadherin | Cadherins are calcium dependent cell adhesion |
| | | cluster | (epithelial) | proteins. They preferentially interact with |
| | | | | themselves in a homophilic manner in connecting |
| | | | | cells; cadherins may thus contribute to the |
| | | | | sorting of heterogeneous cell types. E-cadherin |
| | | | | has a potent invasive suppressor role. It is also |
| | | | | a ligand for integrin alpha-E/beta-7. |
| SLC37A3 | 84255 | Early | Solute carrier family 37 | |
| | | cluster | (glycerol-3-phosphate | |
| | | | transporter), member 3 | |
| PALM2-AKAP2 | 11217 | Early | A kinase (PRKA) anchor protein 2 | Binds to regulatory subunit (RII) of protein |
| | | cluster | | kinase A. May be involved in establishing polarity |
| | | | | in signaling systems or in integrating PKA-RII |
| | | | | isoforms with downstream effectors to capture, |
| | | | | amplify and focus diffuse, trans-cellular signals |
| | | | | carried by cAMP (By similarity). PALM2-AKAP2 mRNA |
| | | | | is a naturally occurring co-transcribed product of |
| | | | | the neighboring PALM2 and AKAP2 genes. The |
| | | | | significance of this co-transcribed mRNA and the |
| | | | | function of its protein product have not yet been |
| | | | | determined. |
| NSMCE1 | 197370 | Early | non-SMC element 1 homolog (S. cerevisiae) | |
| | | cluster | | |
| TSPAN13 | 27075 | 24 h CD4 | Tetraspanin 13 (transmembrane 4 | The protein encoded by this gene is a member of |
| | | | superfamily member 13) | the transmembrane 4 superfamily, also known as the |
| | | | | tetraspanin family. Most of these members are |
| | | | | cell-surface proteins that are characterized by |
| | | | | the presence of four hydrophobic domains. The |
| | | | | proteins mediate signal transduction events that |
| | | | | play a role in the regulation of cell development, |
| | | | | activation, growth and motility. The use of |
| | | | | alternate polyadenylation sites has been found for |
| | | | | this gene. |
| SYTL3 | 94120 | 24 h CD4 | synaptotagmin-like 3 | |
| SFRS8 | 6433 | 6 hr | splicing factor; | This gene encodes a human homolog of Drosophila |
| | | | arginine/serine-rich 8 | splicing regulatory protein. This gene |
| | | | (suppressor-of-white-apricot | autoregulates its expression by control of |
| | | | homolog, Drosophila) | splicing of its first two introns. In addition, it |
| | | | | also regulates the splicing of fibronectin and |
| | | | | CD45 genes. Multiple alternatively spliced |
| | | | | variants have been identified. Two alternatively |
| | | | | spliced variants have been characterized to date. |
| FIP1L1 | 81608 | 6 hr | FIP1 like 1 (S. cerevisiae) | Component of the cleavage and polyadenylation |
| | | | | specificity factor (CPSF) complex that plays a key |
| | | | | role in pre-mRNA 3′-end formation, recognizing the |
| | | | | AAUAAA signal sequence and interacting with |
| | | | | poly(A) polymerase and other factors to bring |
| | | | | about cleavage and poly(A) addition. FIP1L1 |
| | | | | contributes to poly(A) site recognition and |
| | | | | stimulates poly(A) addition. Binds to U-rich RNA |
| | | | | sequence elements surrounding the poly(A) site. |
| | | | | May act to tether poly(A) polymerase to the CPSF |
| | | | | complex. |
| MAML3 | 55534 | 6 hr | mastermind-like 3 (Drosophila) | Acts as a transcriptional coactivator for NOTCH |
| | | | | proteins. Has been shown to amplify NOTCH-induced |
| | | | | transcription of HES1. |
| TRIM4 | 89122 | 6 hr | tripartite motif-containing 4 | The protein encoded by this gene is a member of |
| | | | | the tripartite motif (TRIM) family. The TRIM motif |
| | | | | includes three zinc-binding domains, a RING, a B- |
| | | | | box type 1 and a B-box type 2, and a coiled-coil |
| | | | | region. The protein localizes to cytoplasmic |
| | | | | bodies. Its function has not been identified. |
| | | | | Alternative splicing of this gene generates two |
| | | | | transcript variants. |
| SIAH1 | 6477 | 6 hr | Seven in absentia homolog 1 | E3 ubiquitin ligase protein that mediates |
| | | | (Drosophila) | ubiquitination and subsequent proteasomal |
| | | | | degradation of target proteins. E3 ubiquitin |
| | | | | ligases accept ubiquitin from an E2 ubiquitin- |
| | | | | conjugating enzyme in the form of a thioester and |
| | | | | then directly transfers the ubiquitin to targeted |
| | | | | substrates. Mediates E3 ubiquitin ligase activity |
| | | | | either through direct binding to substrates or by |
| | | | | functioning as the essential RING domain subunit |
| | | | | of larger E3 complexes. Triggers the ubiquitin- |
| | | | | mediated degradation of many substrates, including |
| | | | | proteins involved in transcription regulation |
| | | | | (MYB, POU2AF1, PML and RBBP8), a cell surface |
| | | | | receptor (DCC), cytoplasmic signal transduction |
| | | | | molecules (TIEG1 and NUMB), an antiapoptotic |
| | | | | protein (BAG1), a microtubule motor protein |
| | | | | (KIF22), a protein involved in synaptic vesicle |
| | | | | function in neurons (SYP), a structural protein |
| | | | | (CTNNB1) and SNCAIP. It is thereby involved in |
| | | | | many cellular processes such as apoptosis, tumor |
| | | | | suppression, cell cycle, axon guidance, |
| | | | | transcription regulation, spermatogenesis and TNF- |
| | | | | alpha signaling. Has some overlapping function |
| | | | | with SIAH2. |
| ITPR1 | 3708 | 6 hr | inositol 1,4,5-triphosphate | Intracellular channel that mediates calcium |
| | | | receptor, type 1 | release from the endoplasmic reticulum following |
| | | | | stimulation by inositol 1,4,5-trisphosphate. |
| ITSN2 | 50618 | 12 h 3rep | Intersectin 2 | Adapter protein that may provide indirect link |
| | | | | between the endocytic membrane traffic and the |
| | | | | actin assembly machinery. May regulate the |
| | | | | formation of clathrin-coated vesicles. |
| CLCF1 | 23529 | 12 h 3rep | Cardiotrophin-like cytokine | Cytokine with B-cell stimulating capability. Binds |
| | | | factor 1 (Novel neurotrophin- | to and activates the ILST/gp130 receptor. |
| | | | 1/B-cell stimulating factor-3) | |
| CRLF1 | 9244 | 12 h 3rep | cytokine receptor-like factor 1 | Cytokine receptor subunit, possibly playing a |
| | | | | regulatory role in the immune system and during |
| | | | | fetal development. May be involved in nervous |
| | | | | system development. SUBUNIT: Forms covalently |
| | | | | linked di- and tetramers. Forms a heteromeric |
| | | | | complex with cardiotrophin-like cytokine (CLC); |
| | | | | the CRLF1/CLC complex is a ligand for the ciliary |
| | | | | neurotrophic factor receptor (CNTFR). |
| CLIC5 | 53405 | 24 h 3rep | chloride intracellular channel 5 | Possible chloride ion channel. |
| IGJ | 3512 | 24 h CD4 | Immunoglobulin J polypeptide, | Serves to link two monomer units of either IgM or |
| | | | linker protein for | IgA. In the case of IgM, the J chain-joined dimer |
| | | | immunoglobulin alpha and mu | is a nucleating unit for the IgM pentamer, and in |
| | | | polypeptides | the case of IgA it induces larger polymers. It |
| | | | | also help to bind these immunoglobulins to |
| | | | | secretory component. |
| NFKBIZ | 64332 | 24 h CD4 | nuclear factor of kappa light | This gene is a member of the ankyrin-repeat family |
| | | | polypeptide gene enhancer in B- | and is induced by lipopolysaccharide (LPS). The C- |
| | | | cells inhibitor, zeta | terminal portion of the encoded product which |
| | | | | contains the ankyrin repeats, shares high sequence |
| | | | | similarity with the I kappa B family of proteins. |
| | | | | The latter are known to play a role in |
| | | | | inflammatory responses to LPS by their interaction |
| | | | | with NF-B proteins through ankyrin-repeat domains. |
| | | | | Studies in mouse indicate that this gene product |
| | | | | is one of the nuclear I kappa B proteins and an |
| | | | | activator of IL-6 production. |
| DLC1 | 10395 | 24 h CD4 | deleted in liver cancer 1 | Functions as a GTPase-activating protein specific |
| | | | | for Rho and an activator of PLCD1 in vivo and |
| | | | | induces morphological changes and detachment |
| | | | | through cytoskeletal reorganization (By |
| | | | | similarity). |
| GBP5 | 115362 | 24 h CD8 | Guanylate binding protein 5 | |
| PEG10 | 23089 | 24 h non | paternally expressed 10 | |
| | | T-cells | | |
| HOMER2 | 9455 | 24 h non | homer homolog 2 (Drosophila) | Postsynaptic density scaffolding protein. Binds |
| | | T-cells | | and cross-links cytoplasmic regions of GRM1, GRM5, |
| | | | | ITPR1, DNM3, RYR1, RYR2, SHANK1 and SHANK3. By |
| | | | | physically linking GRM1 and GRM5 with ER- |
| | | | | associated ITPR1 receptors it aids the coupling of |
| | | | | surface receptors to intracellular calcium |
| | | | | release. Isoforms can be differently regulated and |
| | | | | may play an important role in maintaining the |
| | | | | plasticity at glutamatergic synapses. |
| ZBTB8 | 127557 | 24 h non | zinc finger and BTB domain | May be involved in transcriptional regulation. |
| | | T-cells | containing 8 | |
| MOBKL2C | 148932 | 24 h non | MOB1, Mps One Binder kinase | May regulate the activity of kinases (By |
| | | T-cells | activator-like 2C (yeast) | similarity). |
| EDG3 | 1903 | Terry | endothelial differentiation, | Receptor for the lysosphingolipid sphingosine 1- |
| | | Speed | sphingolipid G-protein-coupled | phosphate (S1P). S1P is a bioactive |
| | | | receptor, 3 | lysophospholipid that elicits diverse |
| | | | | physiological effect on most types of cells and |
| | | | | tissues. When expressed in rat HTC4 hepatoma |
| | | | | cells, is capable of mediating S1P-induced cell |
| | | | | proliferation and suppression of apoptosis. |
| MELK | 9833 | Terry | maternal embryonic leucine | Phosphorylates ZNF622 and may contribute to its |
| | | Speed | zipper kinase | redirection to the nucleus. May be involved in the |
| | | | | inhibition of spliceosome assembly during mitosis. |
| PHC3 | 80012 | Late | polyhomeotic like 3 (Drosophila) | Polycomb group (PcG) protein. PcG proteins form |
| | | cluster | | multiprotein complexes, which are required to |
| | | | | maintain the transcriptional repressive state of |
| | | | | homeotic genes throughout development. |
| | | | | Transcriptional repressors that maintain the |
| | | | | silenced state of Hox genes in a stable and |
| | | | | heritable manner. |
| TTC3 | 7267 | Late | tetratricopeptide repeat domain 3 | |
| | | cluster | | |
| KLK1 | 3816 | Alternative | kallikrein 1, | Kallikreins are a subgroup of serine proteases |
| | | CDF | renal/pancreas/salivary | having diverse physiological functions. Growing |
| | | | | evidence suggests that many kallikreins are |
| | | | | implicated in carcinogenesis and some have |
| | | | | potential as novel cancer and other disease |
| | | | | biomarkers. This gene is one of the fifteen |
| | | | | kallikrein subfamily members located in a cluster |
| | | | | on chromosome 19. This protein is functionally |
| | | | | conserved in its capacity to release the |
| | | | | vasoactive peptide, Lys-bradykinin, from low |
| | | | | molecular weight kininogen. |
| KCNV2 | 169522 | Alternative | potassium channel, subfamily V, | Voltage-gated potassium (Kv) channels represent |
| | | CDF | member 2 | the most complex class of voltage-gated ion |
| | | | | channels from both functional and structural |
| | | | | standpoints. Their diverse functions include |
| | | | | regulating neurotransmitter release, heart rate, |
| | | | | insulin secretion, neuronal excitability, |
| | | | | epithelial electrolyte transport, smooth muscle |
| | | | | contraction, and cell volume. This gene encodes a |
| | | | | member of the potassium voltage-gated channel |
| | | | | subfamily V. This member is identified as a |
| | | | | ‘silent subunit’, and it does not form |
| | | | | homomultimers, but forms heteromultimers with |
| | | | | several other subfamily members. Through |
| | | | | obligatory heteromerization, it exerts a function- |
| | | | | altering effect on other potassium channel |
| | | | | subunits. This protein is strongly expressed in |
| | | | | pancreas and has a weaker expression in several |
| | | | | other tissues. |
| GBP1 | 2633 | Terry | guanylate binding protein 1 | Guanylate binding protein expression is induced by |
| | | Speed | | interferon. Guanylate binding proteins are |
| | | | | characterized by their ability to specifically |
| | | | | bind guanine nucleotides (GMP, GDP, and GTP) and |
| | | | | are distinguished from the GTP-binding proteins by |
| | | | | the presence of 2 binding motifs rather than 3 |
| IL1F9 | 56300 | Terry | interleukin 1 family, member 9 | The protein encoded by this gene is a member of |
| | | Speed | | the interleukin 1 cytokine family. The activity of |
| | | | | this cytokine is mediated by interleukin 1 |
| | | | | receptor-like 2 (IL1RL2/IL1R-rp2), and is |
| | | | | specifically inhibited by interleukin 1 family, |
| | | | | member 5 (IL1F5/IL-1 delta). Interferon-gamma, |
| | | | | tumor necrosis factor-alpha and interleukin 1, |
| | | | | beta (IL1B) are reported to stimulate the |
| | | | | expression of this cytokine in keratinocytes. The |
| | | | | expression of this cytokine in keratinocytes can |
| | | | | also be induced by a contact hypersensitivity |
| | | | | reaction or herpes simplex virus infection. This |
| | | | | gene and eight other interleukin 1 family genes |
| | | | | form a cytokine gene cluster on chromosome 2. |
| SEL1 | 85465 | Terry | selenoprotein I | The product of this gene belongs to the family of |
| | | Speed | | selenoproteins. Selenoproteins contain the rare |
| | | | | twenty-first amino acid, selenocysteine (sec). |
| | | | | These proteins lack common amino acid sequence |
| | | | | motifs, but the 3-prime untranslated regions of |
| | | | | selenoprotein genes have a common stem-loop |
| | | | | structure, the sec insertion sequence (SECIS), |
| | | | | that is necessary for the recognition of UGA as a |
| | | | | sec codon rather than as a stop signal. |
| | | | | Selenoproteins are thought to be responsible for |
| | | | | most biomedical effects of dietary selenium and |
| | | | | are essential to mammals. This gene encodes a |
| | | | | selenoprotein I. |
| IL1R2 | 7850 | Terry | interleukin 1 receptor, type II | The protein encoded by this gene is a cytokine |
| | | Speed | | receptor that belongs to the interleukin 1 |
| | | | | receptor family. This protein binds interleukin |
| | | | | alpha (IL1A), interleukin beta (IL1B), and |
| | | | | interleukin 1 receptor, type I(IL1R1/IL1RA), and |
| | | | | acts as a decoy receptor that inhibits the |
| | | | | activity of its ligands. Interleukin 4 (IL4) is |
| | | | | reported to antagonize the activity of interleukin |
| | | | | 1 by inducing the expression and release of this |
| | | | | cytokine. This gene and three other genes form a |
| | | | | cytokine receptor gene cluster on chromosome 2q12. |
| | | | | Two alternatively spliced transcript variants |
| | | | | encoding the same protein have been reported. |
| IFI44L | 10561 | Terry | interferon-induced protein 44 | |
| | | Speed | | |
| LIX1L | 128077 | Terry | Lix1 homolog (mouse) like | |
| | | Speed | | |

Biologically active fragments of a nucleic acid molecule or gene are also within the scope of the invention. The term “fragment” means a portion of the entire molecule. The size of the fragment is limited only in that it must retain a biological activity of the full-length molecule, such as the ability to be expressed in an allergic animal at a different level to that in a non-allergic animal.

As used herein, an “activated gene” means that the mRNA corresponding to the gene of interest is actively being transcribed in a mammal and/or that the protein encoded by the gene can be detected in the mammal. Thus, the term “level of expression” refers to the amount of mRNA being transcribed from the nucleic acid or gene or, in some embodiments described infra, the amount of protein which can be detected in the mammal.

It is known that disorders, such as allergic disorders, may be associated with the upregulation or down regulation of a gene. Whether the gene is upregulated or down-regulated will depend on factors such as the specific gene and the disorder. Some disorders are associated with a group of genes in which some of the genes are upregulated and others are down-regulated. Thus, genes of the invention may be upregulated, i.e. have a higher level of expression, in a cell of an allergic animal compared to the level in a cell of a non-atopic animal, which means that more of the mRNA and/or protein corresponding to the gene is present in a cell of an allergic animal compared to the level in a cell of a non-atopic animal. Alternatively, the genes of the invention may be down-regulated. Where more than one gene of the invention is associated with an allergic disorder, some of the genes may be upregulated while others are down-regulated.

It will be apparent to a person skilled in the art that many of the methods provided by the present invention require not only a measurement of the level of expression of the nucleic acids described herein (“genes of interest”) in a test subject, but also a comparison to the levels of expression in a healthy or normal subject. Accordingly, a cell or gene from a normal or non-allergic mammal is, in some embodiments, also contacted with the same allergen to produce a “known standard”. Thus, a known standard may be derived from an established data set that has been generated from healthy or normal subjects by the same methods described supra.

In the present context, the term “healthy subject” or “non-allergic mammal” shall be taken to mean a mammalian subject that is known not to suffer from an allergic disorder, such knowledge being derived from clinical data on the subject. The term “normal subject” shall be taken to mean a subject individual having a normal expression level or amount of the proteins encoded by the genes of interest in a particular sample derived from said subject. As will be known to those skilled in the art, data obtained from a sufficiently large sample of subjects will normalize, allowing the generation of a data set for determining the average level of a particular parameter. Accordingly, the “known standard” can be determined for any population of subjects, and for any sample derived from said subjects, for subsequent comparison to the relative amounts of the mRNA or protein in a sample being assayed i.e. from a test subject. Where such normalized data sets are relied upon, internal controls are preferably included in each assay conducted to control for variation.

The level of expression or expression pattern of a nucleic acid or gene may be determined by any method known in the art, including the determination of the level of mRNA and/or protein. “Differential expression,” or grammatical equivalents as used herein, refers to qualitative or quantitative differences in the temporal and/or cellular gene expression patterns within and among cells and tissue. The degree to which expression differs need only be large enough to measure via standard characterization techniques as outlined below, such as by use of Affymetrix GeneChip™ expression arrays, Lockhart, Nature Biotechnology 14:1675-1680 (1996), hereby expressly incorporated by reference. Other techniques include, but are not limited to, in situ hybridization, northern blotting techniques, RNase protection assays, quantitative reverse transcriptase PCR (RT-PCR) analysis (such as, for example, performed on laser capture microdissected samples), and microarray technology, such as, for example, using tissue microarrays probed with nucleic acid probes, or nucleic acid microarrays (ie. RNA microarrays or amplified DNA microarrays) microarrays probed with nucleic acid probes.

Although DNA or RNA encoding the genes of interest can be detected, of particular interest are methods wherein an mRNA expressed by the genes of interest are detected, measured or evaluated. Probes to detect mRNA are a nucleotide/deoxynucleotide probe that is complementary to and hybridizes with the mRNA and includes, but is not limited to, oligonucleotides, cDNA or RNA. Probes also should contain a detectable label, as defined herein. In one method the mRNA is detected after immobilizing the nucleic acid to be examined on a solid support such as nylon membranes and hybridizing the probe with the sample. Following washing to remove the non-specifically bound probe, the label is detected. In another method detection of the mRNA is performed in situ. In this method permeablized cells or tissue samples are contacted with a detectably labelled nucleic acid probe for sufficient time to allow the probe to hybridize with the target mRNA. Following washing to remove the non-specifically bound probe, the label is detected. For example a digoxygenin labelled riboprobe (RNA probe) that is complementary to the mRNA encoding a protein of interest is detected by binding the digoxygenin with an anti-digoxygenin secondary antibody and developed with nitro blue tetrazolium and 5-bromo-4-chloro-3-indoyl phosphate.

Whilst the probes may comprise double-stranded or single-stranded nucleic acid, single-stranded probes are preferred because they do not require melting prior to use in hybridizations. On the other hand, longer probes are also preferred because they can be used at higher hybridization stringency than shorter probes and may produce lower background hybridization than shorter probes.

Recommended pre-requisites for selecting oligonucleotide probes, particularly with respect to probes suitable for microarray technology, are described in detail by Lockhart et al., 1996, Nature Biotech, 14, 1675-1680.

The nucleic acid probe may comprise a nucleotide sequence that is within the coding strand of a gene of interest as listed in Table 1. Such “sense” probes are useful for detecting RNA by amplification procedures, such as, for example, polymerase chain reaction (PCR), and more preferably, quantitative PCR or reverse transcription polymerase chain reaction (RT-PCR). Alternatively, “sense” probes; may be expressed to produce polypeptides or immunologically active derivatives thereof that are useful for detecting the expressed protein in samples.

“Polymerase chain reaction,” or “PCR,” as used herein generally refers to a method for amplification of a desired nucleotide sequence in vitro, as described in U.S. Pat. No. 4,683,195. In general, the PCR method involves repeated cycles of primer extension synthesis in the presence of PCR reagents, using two oligonucleotide primers capable of hybridizing preferentially to a template nucleic acid. Typically, the primers used in the PCR method will be complementary to nucleotide sequences within the template at both ends of or flanking the nucleotide sequence to be amplified, although primers complementary to the nucleotide sequence to be amplified also may be used. See Wang, et al., in PCR Protocols, pp. 70-75 (Academic Press, 1990); Ochman, et al., in PCR Protocols, pp. 219-227; Triglia, et al., Nucl. Acids Res. 16:8186 (1988).

PCR may also be used to determine whether a specific sequence is present, by using a primer that will specifically bind to the desired sequence, where the presence of an amplification product is indicative that a specific binding complex was formed. Alternatively, the amplified sample can be fractionated by electrophoresis, e.g. capillary or gel electrophoresis, transferred to a suitable support, e.g. nitrocellulose, and then probed with a fragment of the template sequence. Detection of mRNA having the subject sequence is indicative of activation of the gene.

“Oligonucleotides” or “oligonucleotide probes” are short-length, single- or double-stranded polydeoxynucleotides that are chemically synthesised by known methods (involving, for example, triester, phosphoramidite, or phosphonate chemistry), such as described by Engels, et al., Agnew. Chem. Int. Ed. Engl. 28:716-734 (1989). Typically they are then purified, for example, by polyacrylamide gel electrophoresis. Oligonucleotide probes of the invention are DNA molecules that are sufficiently complementary to regions of contiguous nucleic acid residues within the allergy-associated gene nucleic acid to hybridise thereto, preferably under high stringency conditions. Defining appropriate hybridisation conditions is within the skill of the art. See e.g., Maniatis et al., DNA Cloning, vols. I and II. Nucleic Acid Hybridisation. However, briefly, “stringent conditions” for hybridisation or annealing of nucleic acid molecules are those that (1) employ low ionic strength and high temperature for washing, for example, 0.015M NaCl/0.0015M sodium citrate/0.1% sodium dodecyl sulfate (SDS) at 50° C., or (2) employ during hybridisation a denaturing agent such as formamide, for example, 50% (vol/vol) formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C. Another example is use of 50% formamide, 5×SSC (0.75N NaCl, 0.075M sodium citrate), 50 mM sodium phosphate (pH 6.8), 0.1% sodium pyrophosphate, 5×Denhardt's solution, sonicated salmon sperm DNA (50 μg/mL), 0.1% SDS, and 10% dextran sulfate at 42° C., with washes at 42° C. in 0.2×SSC and 0.1% SDS.

Exemplary probes include oligomers that are at least about 15 nucleic acid residues long and that are selected from any 15 or more contiguous residues of DNA of the present invention. Preferably, oligomeric probes used in the practice of the present invention are at least about 20 nucleic acid residues long. The present invention also contemplates oligomeric probes that are 150 nucleic acid residues long or longer. Those of ordinary skill in the art realise that nucleic hybridisation conditions for achieving the hybridisation of a probe of a particular length to polynucleotides of the present invention can readily be determined. Such manipulations to achieve optimal hybridisation conditions for probes of varying lengths are well known in the art. See, e.g., Sambrook et al., Molecular Cloning: A Laboratory Manual, Second Edition, Cold Spring Harbor (1989), incorporated herein by reference.

As used herein, the term “PCR reagents” refers to the chemicals, apart from the template nucleic acid sequence, needed to perform the PCR process. These chemicals generally consist of five classes of components: (i) an aqueous buffer, (ii) a water soluble magnesium salt, (iii) at least four deoxyribonucleotide triphosphates (dNTPs), (iv) oligonucleotide primers (normally two primers for each template sequence, the sequences defining the 5′ ends of the two complementary strands of the double-stranded template sequence), and (v) a polynucleotide polymerase, preferably a DNA polymerase, more preferably a thermostable DNA polymerase, ie a DNA polymerase which can tolerate temperatures between 90° C. and 100° C. for a total time of at least 10 minutes without losing more than about half its activity.

The four conventional dNTPs are thymidine triphosphate (dTTP), deoxyadenosine triphosphate (dATP), deoxycitidine triphosphate (dCTP), and deoxyguanosine triphosphate (dGTP). These conventional deoxyribonucleotide triphosphates may be supplemented or replaced by dNTPs containing base analogues which Watson-Crick base pair like the conventional four bases, e.g. deoxyuridine triphosphate (dUTP).

A detectable label may be included in an amplification reaction. Biotin-labelled nucleotides can be incorporated into DNA or RNA by such techniques as nick translation, chemical and enzymatic means, and the like. The biotinylated probes are detected after hybridisation, using indicating means such as avidin/streptavidin, fluorescent-labelling agents, enzymes, colloidal gold conjugates, and the like. Nucleic acids may also be labelled with other fluorescent compounds, with immunodetectable fluorescent derivatives, with biotin analogues, and the like. Nucleic acids may also be labelled by means of attachment to a protein. Nucleic acids cross-linked to radioactive or fluorescent histone single-stranded binding protein may also be used. Those of ordinary skill in the art will recognise that there are other suitable methods for detecting oligomeric probes and other suitable detectable labels that are available for use in the practice of the present invention. Moreover, fluorescent residues can be incorporated into oligonucleotides during chemical synthesis. Preferably, oligomeric probes of the present invention are labelled to render them readily detectable. Detectable labels may be any species or moiety that may be detected either visually or with the aid of an instrument.

Suitable labels include fluorochromes, e.g. fluorescein isothiocyanate (FITC), rhodamine, Texas Red, phycoerythrin, allophycocyanin, 6-carboxyfluorexcein (6-FAM), 2′,7′-dimethoxy-4′,5′-dichloro-6-carboxyfluorescein (JOE), 6-carboxy-X-rhodamine(ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, eg. ³²P, ³⁵S, ³H, as well as others. Another group of fluorescent compounds are the naphthylamines, having an amino group in the alpha or beta position. Included among such naphthylamino compounds are 1-dimethylaminonaphthyl-5-sulfonate, 1-anilino-8-naphthalene sulfonate and 2-p-touidinyl-6-naphthalene sulfonate. Other dyes include 3-phenyl-7-isocyanatocoumarin, acridines, such as 9-isothiocyanatoacridine acridine orange; N-(p-(2-benzoaxazolyl)phenyl)maleimide; benzoxadiazoles, stilbenes, pyrenes, and the like. Most preferably, the fluorescent compounds are selected from the group consisting of VIC, carboxy fluorescein (FAM), Lightcycler® 640 and Cy5.

The label may be a two stage system, where the amplified DNA is conjugated to biotin, haptens, or the like having a high affinity binding partner, e.g. avidin, specific antibodies, etc., where the binding partner is conjugated to a detectable label. The label may be conjugated to one or both of the primers. Alternatively, the pool of nucleotides used in the amplification is labelled, so as to incorporate the label into the amplification product.

RT-PCR is a form of PCR which can amplify a known mRNA sequence using a reverse transcriptase to convert the mRNA to cDNA prior to traditional PCR. In its simplest implementation, aliquots are removed from the PCR every couple of cycles beginning at a point where product is undetectable (typically about cycle 20) and extending through the entire exponential phase. Products are then resolved electrophoretically and quantitated by densitometry, fluorescence or phosphorimaging. Alternatively, a fluorescent signal can be used to report formation of PCR product as each cycle of the amplification proceeds, coupled with an automated PCR/fluorescent detection system (Heid C. A., Stevens J., Livak K. J., Williams P. M. Real time quantitative PCR. Genome Res. 1996; 6:986-994). Suitable detection systems for real-time RT-PCR include SYBR®Green (Molecular Beacons), Scorpions® (Molecular Probes), and TaqMan® (Applied Biosystems).

In a particularly preferred embodiment the present invention utilises a combined PCR and hybridisation probing system so as to make the most of the closed tube or homogenous assay systems such as the use of FRET probes as disclosed in U.S. Pat. Nos. 6,140,054; 6,174,670, the entirety of which are also incorporated herein by reference. In one of its simplest configurations, the FRET or “fluorescent resonance energy transfer” approach employs two oligonucleotides which bind to adjacent sites on the same strand of the nucleic acid being amplified. One oligonucleotide is labelled with a donor fluorophore which absorbs light at a first wavelength and emits light in response, and the second is labelled with an acceptor fluorophore which is capable of fluorescence in response to the emitted light of the first donor (but not substantially by the light source exciting the first donor, and whose emission can be distinguished from that of the first fluorophore). In this configuration, the second or acceptor fluorophore shows a substantial increase in fluorescence when it is in close proximity to the first or donor fluorophore, such as occurs when the two oligonucleotides come in close proximity when they hybridise to adjacent sites on the nucleic acid being amplified (for example in the annealing phase of PCR) forming a fluorogenic complex. As more of the nucleic acid being amplified accumulates, so more of the fluorogenic complex can be formed and there is an increase in the fluorescence from the acceptor probe, and this can be measured. Hence the method allows detection of the amount of product as it is being formed. In another simple embodiment, and as applies to use of FRET probes in PCR based assays, one of the labelled oligonucleotides may also be a primer used for PCR. In this configuration, the labelled PCR primer is part of the DNA strand to which the second labelled oligonucleotide hybridises, as described by Neoh et al (J Clin Path 1999; 52:766-769.), von Ahsen et al (Clin Chem 2000; 46:156-161), the entirety of which are encompassed by reference.

It will be appreciated by those of skill in the art that amplification and detection of amplification with hybridisation probes can be conducted in two separate phases, for example by carrying out PCR amplification first, and then adding hybridisation probes under such conditions as to measure the amount of nucleic acid which has been amplified. However, a preferred embodiment of the present invention utilises a combined PCR and hybridisation probing system so as to make the most of the closed tube or homogenous assay systems and is carried out on a Roche Lightcycler® or other similarly specified or appropriately configured instrument.

Such systems would also be adaptable to the detection methods described here. Those skilled in the art will appreciate that such probes can be used for allele discrimination if appropriately designed for the detection of point-mutation(s), in addition to deletion(s) and insertion(s). Alternatively or in addition, the unlabelled PCR primers may be designed for allele discrimination by methods well known to those skilled in the art (Ausubel 1989-1999).

It will also be appreciated by those skilled in the art that detection of amplification in homogenous and/or closed tubes can be carried out using numerous means in the art, for example using TaqMan® hybridisation probes in the PCR reaction and measurement of fluorescence specific for the target nucleic acids once sufficient amplification has taken place.

Although those skilled in the art will be aware that other similar quantitative “real-time” and homogenous nucleic acid amplification/detection systems exist such as those based on the TaqMan approach (U.S. Pat. Nos. 5,538,848 and 5,691,146), fluorescence polarisation assays (eg Gibson et al., Clin Chem, 1997; 43: 1336-1341), and the Invader assay (eg Agarwal P et al., Diagn Mol Pathol 2000 September; 9(3): 158-164; Ryan D et al, Mol. Diagn 1999 June; 4(2): 135-144). Such systems would also be adaptable to use the invention described, enabling real-time monitoring of nucleic acid amplification.

Northern blot analysis involves fractionating RNA species on the basis of size by denaturing gel electrophoresis followed by transfer of the RNA onto a membrane by capillary, vacuum or pressure blotting (Sambrook, J., Fritsch, E. F. & Maniatis, T. (1989) Molecular Cloning: A Laboratory Manual, 2nd ed. Cold Spring Harbor Laboratory, Cold Spring Harbor, N.Y.). The RNA may be bound to the membrane in an apparent noncovalent interaction via exposure to short wave ultraviolet light or by heating at 80° C. in a vacuum oven. RNA sequences of interest are detected on the blot by hybridization to an oligonucleotide probe. Probes for Northern blot detection generally contain full or partial cDNA sequences and may be labelled by enzymatic incorporation of radiolabeled (usually ³²P or ³³P) nucleotides or with nucleotides conjugated to haptens such as biotin for subsequent chemiluminescent detection. After probe hybridization and washing to remove non-specific label, the hybridization signal is generally detected by exposing blots to X-ray film or phosphor storage plates, after prior incubation with chemiluminescent substrates if necessary. The resulting band identified by the probe indicates the size of the mRNA, and the intensity of the band corresponds to the relative abundance. Autoradiograph band intensities may be quantitated by densitometry, by direct measurement of hybridized radiolabeled probe via storage phosphor imaging or by scintillation counting of excised bands.

The RNase protection assay (RPA) operates on the same principle as a Northern blot as it involves hybridization of a labeled probe to a target mRNA. However, in the RPA, hybridization takes place in a solution containing both a labeled antisense RNA probe and the target mRNA without prior gel fractionation or blotting (Azrolan N., Breslow J. L. A solution hybridization/RNase protection assay with riboprobes to determine absolute levels of apo B, apo A-I and apo E mRNA in human hepatoma cell lines. J. Lipid Res. 1990; 31:1141-1146; Sambrook, J., Fritsch, E. F. & Maniatis, T. (1989) Molecular Cloning: A Laboratory Manual, 2nd ed. Cold Spring Harbor Laboratory, Cold Spring Harbor, N.Y.). After incubation for several hours, unhybridized probe and sample RNA are enzymatically degraded and the remaining hybrids are electrophoresed through a denaturing polyacrylamide gel and visualized by autoradiography or phosphorimaging. Alternatively, the RNase-resistant hybrids may be precipitated and bound to filters for direct quantitation by scintillation counting (Melton D. A., Krieg P. A., Rebagliati M. R., Maniatis T., Zinn K., Green M. R. Efficient in vitro synthesis of biologically active RNA and DNA hybridization probes from plasmids containing a bacteriophage SP6 promoter. Nucleic Acids Res. 1984; 12:7035-7056). Furthermore, by performing titration reactions with unlabeled RNA transcripts corresponding to the mRNA sense strand, absolute RNA levels can be determined.

For high throughput screening of large numbers of samples, such as, for example, public health screening of subjects, particularly human subjects, having a higher risk of developing allergies, microarray technology is a preferred assay format.

In accordance with such high throughput formats, techniques for producing immobilised arrays of DNA molecules have been described in the art. Generally, most prior art methods describe how to synthesise single-stranded nucleic acid molecule arrays, using for example masking techniques to build up various permutations of sequences at the various discrete positions on the solid substrate. U.S. Pat. No. 5,837,832, the contents of which are incorporated herein by reference, describes an improved method for producing DNA arrays immobilised to silicon substrates based on very large scale integration technology. In particular, U.S. Pat. No. 5,837,832 describes a strategy called “tiling” to synthesize specific sets of probes at spatially-defined locations on a substrate Which are used to produce the immobilised DNA arrays. U.S. Pat. No. 5,837,832 also provides references for earlier techniques that may also be used.

Thus DNA are synthesised in situ on the surface of the substrate. However, DNA may also be printed directly onto the substrate using for example robotic devices equipped with either pins or piezo electric devices.

The plurality of polynucleotide sequences are typically immobilised onto or in discrete regions of a solid substrate. The substrate is porous to allow immobilisation within the substrate or substantially non-porous, in which case the library sequences are typically immobilised on the surface of the substrate. The solid substrate is made of any material to which polypeptides can bind, either directly or indirectly. Examples of suitable solid substrates include flat glass, silicon wafers, mica, ceramics and organic polymers such as plastics, including polystyrene and polymethacrylate. It may also be possible to use semi-permeable membranes such as nitrocellulose or nylon membranes, which are widely available. The semi-permeable membranes are mounted on a more robust solid surface such as glass. The surfaces may optionally be coated with a layer of metal, such as gold, platinum or other transition metal. A particular example of a suitable solid substrate is the commercially available BIACore™ chip (Pharmacia Biosensors).

For high throughput screening, the sample or probe will generally comprise an array of nucleic acids on glass or other solid matrix, such as, for example, as described in WO 96/17958. Techniques for producing high density arrays are described, for example, by Fodor et al., 1991, Science, 767-773 and in U.S. Pat. No. 5,143,854. Typical protocols for other assay formats can be found, for example in Current Protocols In Molecular Biology, Unit 2 (Northern Blotting), Unit 4 (Southern Blotting), and Unit 18 (PCR Analysis), Frederick M. Ausubul et al, (ed)., 1995.

The detection means according to this aspect of the invention may be any nucleic acid-based detection means such as, for example, nucleic acid hybridization or amplification reaction (eg. PCR), a nucleic acid sequence-based amplification (NASBA) system, inverse polymerase chain reaction (iPCR), in situ polymerase chain reaction, or RT-PCR, amongst others.

The probe can be labelled with a reporter molecule capable of producing an identifiable signal (eg., a radioisotope such as ³²P or ³⁵S, or a fluorescent or biotinylated molecule). According to this embodiment, those skilled in the art will be aware that the detection of said reporter molecule provides for identification of the probe and that, following the hybridization reaction, the detection of the corresponding nucleotide sequences in the sample is facilitated. Additional probes can be used to confirm the assay results obtained using a single probe.

Wherein the detection means is an amplification reaction such as, for example, a polymerase chain reaction or a nucleic acid sequence-based amplification (NASBA) system or a variant thereof, one or more nucleic acid probes molecules of at least about 20 contiguous nucleotides in length is hybridized to mRNA encoding a protein, or alternatively, hybridized to cDNA or cRNA produced from said mRNA, and nucleic acid copies of the template are enzymatically-amplified.

In one format, PCR provides for the hybridization of non-complementary probes to different strands of a double-stranded nucleic acid template molecule (ie. a DNA/RNA, RNA/RNA or DNA/DNA template), such that the hybridized probes are positioned to facilitate the 5′- to 3′ synthesis of nucleic acid in the intervening region, under the control of a thermostable DNA polymerase enzyme. In accordance with this embodiment, one sense probe and one antisense probe as described herein would be used to amplify DNA from the hybrid RNA/DNA template or cDNA.

In the present context, the cDNA would generally be produced by reverse transcription of mRNA present in the sample being tested (ie. RT-PCR). RT-PCR is particularly useful when it is desirable to determine expression of a gene of interest. It is also known to those skilled in the art to use mRNA/DNA hybrid molecules as a template for such amplification reactions, and, as a consequence, first strand cDNA synthesis is all that is required to be performed prior to the amplification reaction.

Variations of the embodiments described herein are described in detail by McPherson et al., PCR: A Practical Approach. (series eds, D. Rickwood and B. D. Hames), IRL Press Limited, Oxford. pp 1-253, 1991.

Another method of detecting the amount of mRNA transcribed from a gene involves using specific nucleic acid microarrays and microchip technology. A microarray is a tool for analysing gene expression and typically consists of a small membrane or glass slide onto which samples of many nucleic acids molecules have been arranged in a regular pattern. A nucleic acid microarray works by exploiting the ability of a given mRNA molecule to hybridise to the DNA template from which it originated. By using an array containing many nucleic acid samples, the expression levels of numerous genes can be determined by measuring the amount of mRNA bound to each site on the array. With the aid of a computer, the amount of mRNA bound to each site can be precisely measured. Various labels may be employed, most commonly radioisotopes, particularly ³²P. However, other techniques may also be employed, such as using biotin-modified nucleotides for introduction into a polynucleotide. The biotin then serves as the site for binding to avidin or antibodies, which may be labelled with a wide-variety of labels, such as radioisotopes, fluorophores, chromophores, or the like. Keller, et al., DNA Probes, pp. 149-213 (Stockton Press, 1989). Alternatively, antibodies may be employed that can recognise specific duplexes, including DNA duplexes, RNA duplexes, and DNA-RNA hybrid duplexes or DNA-protein duplexes. The antibodies in turn may be labelled and the assay may be carried out where the duplex is bound to a surface, so that upon the formation of duplex on the surface, the presence of antibody bound to the duplex can be detected.

In one embodiment of the invention an initial procedure involves the manufacture of the oligonucleotide matrice or microchip. These contain a selection of immobilized synthetic oligomers, said oligomers synthesized so as to contain complementary sequences for desired portions of transcription factor DNA. The oligomers are then hybridized with cloned or PCR amplified transcription factor nucleic acids, said hybridization occurring under stringent conditions, outlined above. The high stringency conditions ensure that only perfect or near perfect matches between the sequence embedded in the microchip and the target sequence will occur during hybridization.

After each initial hybridization, the chip is washed to remove most mismatched fragments. The reaction mixture is then denatured to remove the bound DNA fragments, which are subsequently labelled with a fluorescent marker. A second round of hybridization with the labelled DNA fragments is then carried out on sequence microchips containing a different set of immobilized oligonucleotides. These fragments first may be cleaved into smaller lengths. The different set of immobilized nucleotides may contain oligonucleotides needed for whole sequencing, partial sequencing, sequencing comparison, or sequence identification. Ultimately, the fluorescence from this second hybridization step can be detected by an epifluorescence microscope coupled to a CCD camera. (See U.S. Pat. No. 5,851,772 incorporated herein by reference).

Another method of detecting expression of a molecule of the invention is to use microfluidics technology. Microfluidics devices comprise fluidic channels of <1 μm and use an electrical field to control the flow rate of the fluid. Microfluidics technology can be applied to nucleic acid or protein microarrays using networks of microfluidics channels plus an integrated pump (Lenigk R, Liu R H, Athavale M, Chen Z, Ganser D, Yang J, Rauch C, Liu Y, Chan B, Yu H. Ray M, Marrero R, Grodzinski P: Plastic biochannel hybridization devices: a new concept for microfluidic DNA arrays. Anal Biochem 2002, 311:40-49; Wang Y, Vaidya B, Farquar H D, Stryjewski W. Hammer R P, McCarley R L, Soper S A, Cheng Y W, Barany F: Microarrays assembled in microfluidic chips fabricated from poly(methyl methacrylate) for the detection of low-abundant DNA mutations. Anal Chem 2003, 75:1130-1140; Barry R, Scrivener E, Soloviev M, Terrett J: Chip-Based Proteomics Technologies. Int Genomic/Proteomic Technology 2002, 14-22; Scrivener E, Barry R, Platt A, Calvert R, Masih G, Hextall P, Soloviev M, Terrett J: Peptidomics: A new approach to affinity protein microarrays. Proteomics 2003, 3:122-128; Barry R, Diggle T, Terrett J, Soloviev M: Competitive assay formats for high-throughput affinity arrays. J Biomol Screen 2003, 8:257-263). Alternatively, cavitation microstreaming, which involves the use of a sound field to induce the vibration of air-bubbles (at a solid surface) present within a fluid, can be used (Liu R H, Lenigk R, Druyor-Sanchez R L, Yang J, Grodzinski P: Hybridization enhancement using cavitation microstreaming. Anal Chem 2003, 75:1911-1917).

In some embodiments, the invention provides an “expression pattern” from normal or healthy subjects as defined herein, which indicates the level or amount of gene expression of one or more genes of interest in a normal sample. This is often referred to as a “standard expression pattern” i.e. a pattern of one or more genes of interest taken from a normal or non-atopic subject. By comparing the expression patterns in samples taken from test subjects with these standard expression patterns, the test subject's susceptibility or pre-disposition to a particular allergic disorder can be determined by locating the presence or absence of an “altered” expression pattern i.e. one that is not the same as the “standard expression pattern”.

The term “known standard pattern” includes patterns derived from healthy cells, advantageously from a similar origin as the source. In some embodiments, the standard pattern is an average of many samples of a certain cell type and/or a certain cellular compartment. In another embodiment, the standard pattern may be derived from a subject prior to the onset of an allergic disease or from cells not affected by the allergic disease. Or, in another embodiment the standard pattern can be an average of the patterns obtained from numerous sources, e.g., the standard pattern may be an average of patterns obtained from 2 or more non-atopic subjects.

The language “aberrant levels” or “abnormal pattern” includes any level, amount, or concentration of an mRNA in a cell, cellular compartment, or organelle which is different to the level of the mRNA of a sample taken from a non-atopic subject.

In some preferred embodiments, the methods of the present invention include the formation of a panel of specific sequences or genes comprising at least CAMK2D and CDH1. Additional panels can be constructed which would include any one of SLC37A3, PALM2-AKAP2, NSMCE1, TSPAN13, SYTL3, SFRS8, FIP1L1, MAML3, TRIM4, SIAH1, ITPR1, ITSN2, CLCF1, CRLF1, CLIC5, IGJ, NFKBIZ, DLC1, GBP5, PEG10, HOMER2, ZBTB8, MOBKL2C, EDG3, MELK, PHC3, TTC3, KLK1, KCNV2, IL1F9, GBP1, SEL1, IL1R2, IFI44L or LIX1L.

In other embodiments, the panel can further comprise one or more specific sequences selected from the group consisting of DACT1, IL17RB, KRT1, LNPEP, MAL, NCOA3, OAZ, PECAM1, PLXDC1, RASGRP3, SLC39A8, XBP1, NDFIP2, RAB27B, GNG8, GJB2 and CISH.

In some embodiments, the “level of expression” of a nucleic acid or gene is determined by detecting the amount of protein encoded by a gene of interest in an allergic subject and comparing it to the amount of nucleic acid or protein in a normal subject. The terms “protein of interst” or “proteins of the invention” refers to the proteins transcribed and translated from the genes of interest or encoded by the genes of interest.

The term “relative amount” or “relative level” as used herein refers to the level, amount or concentration of each nucleic acid (eg mRNA) or protein encoded by one or more of the genes of interest, when normalised or standardised to a known amount of said protein. There are a number of methods known in the art for measuring the relative amount of proteins. For example, immunoassays such as the various types of enzyme linked immunosorbent assays (ELISAs) and radioimmunoassays (RIA) are well known in the art. Other techniques such as Western blotting, dot blotting, FACS analyses, and the like may also be used.

Most preferably, the method of determining the relative amount will be capable of generating quantitative results directly.

In some embodiments, the relative amount of protein in a sample is measured by contacting the sample derived from a subject with an antibody capable of binding to a specific protein or an immunogenic fragment or epitope thereof, and then detecting the formation of an antigen-antibody complex using a detection system.

Preferred detection systems contemplated herein include any known method for detecting proteins or the antibodies bound thereto in a sample isolated from a subject, such as, for example, SDS/PAGE, isoelectric focussing, 2-dimensional gel electrophoresis comprising SDS/PAGE and isoelectric focussing, an immunoassay, a detection based system using an antibody or non-antibody ligand of the protein, such as, for example, a small molecule (e.g. a chemical compound, agonist, antagonist, allosteric modulator, competitive inhibitor, or non-competitive inhibitor, of the protein). In accordance with these embodiments, the antibody or small molecule may be used in any standard solid phase or solution phase format amenable to the detection of proteins. Optical or fluorescent detection, such as, for example, using mass spectrometry, MALDI-TOF, biosensor technology, evanescent fibre optics, or fluorescence resonance energy transfer, is clearly encompassed by the present invention. Detection systems suitable for use in high throughput screening of mass samples, particularly a high throughput spectroscopy resonance method (e.g. MALDI-TOF, electrospray MS or nano-electrospray MS), are particularly contemplated.

Immunoassay formats are particularly preferred, eg., selected from the group consisting of, an immunoblot, a Western blot, a dot blot, an enzyme linked immunosorbent assay (ELISA), radioimmunoassay (RIA), enzyme immunoassay. Modified immunoassays utilizing fluorescence resonance energy transfer (FRET), isotope-coded affinity tags (ICAT), matrix-assisted laser desorption/ionization time of flight (MALDI-TOF), electrospray ionization (ESI), biosensor technology, evanescent fiber-optics technology or protein chip technology are also useful.

Standard solid phase ELISA formats are particularly useful in determining the concentration of a protein from a variety of samples.

Reference herein to antibody or antibodies includes whole polyclonal and monoclonal antibodies, and parts thereof, either alone or conjugated with other moieties. Antibody parts include Fab and F(ab)₂ fragments and single chain antibodies. The antibodies may be made in vivo in suitable laboratory animals, or, in the case of engineered antibodies (Single Chain Antibodies or SCABS, etc) using recombinant DNA techniques in vitro.

Means for preparing and characterizing antibodies are well known in the art. (See; eg., Antibodies: A Laboratory Manual, Cold Spring Harbor Laboratory, 1988, incorporated herein by reference). Conveniently, the antibodies may be prepared against a synthetic peptide based on the protein or peptide encoded by genes such as CAMK2D, CDH1, SLC37A3, PALM2-AKAP2, NSMCE1, TSPAN13, SYTL3, SFRS8, FIP1L1, MAML3, TRIM4, SIAH1, ITPR1, ITSN2, CLCF1, CRLF1, CLIC5, IGJ, NFKBIZ, DLC1, GBP5, PEG10, HOMER2, ZBTB8, MOBKL2C, EDG3, MELK, PHC3, TTC3, KLK1, KCNV2, IL1F9, GBP1, SEL1, IL1R2, IFI44L and LIX1L.

The antibodies used in the detection systems described herein generally bind specifically to their respective targets. The phrase “binds specifically” to a polypeptide means that the binding of the antibody to the proteins of the invention is determinative of the presence of the proteins, in a heterogeneous population of proteins and other biologics. Thus, under designated immunoassay conditions, the specified antibodies bind to a particular protein at least two times the background and more typically more than 10 to 100 times background. Typically, antibodies of the invention bind to a protein of interest with a Kd of at least about 0.1 mM, more usually at least about 1 μM, preferably at least about 0.1 μM, and most preferably at least, 0.0 μM.

In one form of detection system a sample is immobilized onto a solid matrix, such as, for example a polystyrene or polycarbonate microwell or dipstick, a membrane, or a glass support (eg. a glass slide). An antibody that specifically binds a protein of interest is then brought into direct contact with the immobilised sample, and forms a direct bond with any of its target protein present in said sample. The added antibody is generally labelled with a detectable reporter molecule, such as for example, a fluorescent label (eg. FITC or Texas Red) or an enzyme (eg. horseradish peroxidase (HRP)), alkaline phosphatase (AP) or β-galactosidase. Alternatively, or in addition, a second labelled antibody can be used that binds to the first antibody or to the isolated/recombinant antigen. Following washing to remove any unbound antibody or antigen, as appropriate, the label is detected either directly, in the case of a fluorescent label, or through the addition of a substrate, such as for example hydrogen peroxide, TMB, or toluidine, or 5-bromo-4-chloro-3-indol-beta-D-galaotopyranbside (x-gal).

Such ELISA based systems are particularly suitable for quantification of the amount of the proteins of interest in a sample, such as, for example, by calibrating the detection system against known amounts of a standard.

In another form, an ELISA consists of immobilizing an antibody that specifically binds a protein of the invention on a solid matrix, such as, for example, a membrane, a polystyrene or polycarbonate microwell, a polystyrene or polycarbonate dipstick or a glass support. A sample is then brought into physical relation with said antibody, and the antigen in the sample is bound or “captured”. The bound protein can then be detected using a labelled antibody. For example if the protein is captured from a human sample, an anti-human antibody is used to detect the captured protein. Alternatively, a third labelled antibody can be used that binds the second (detecting) antibody.

It will be apparent to the skilled person that the detection systems described herein are amenable to high throughput formats, such as, for example automation of screening processes or a microarray format as described in Mendoza et al., 1999, Biotechniques, 27(4): 778-788. Furthermore, variations of the above described detection system will be apparent to those skilled in the art, such as, for example, a competitive ELISA.

As described elsewhere, Western blotting is also useful for detecting and measuring the relative amounts of proteins of the invention in a sample. In such a detection system protein from a sample is separated using sodium dodecyl sulphate (SDS) polyacrylamide gel electrophoresis (SDS-PAGE) using techniques well known in the art and described in, for example, Scopes (In: Protein Purification: Principles and Practice, Third Edition, Springer Verlag, 1994). Separated proteins are then transferred to a solid support, such as, for example, a, membrane or more specifically PVDF membrane, using methods well known in the art, for example, electrotransfer. This membrane may then be blocked and probed with a labelled antibody or ligand that specifically binds a protein of interest. Alternatively, a labelled secondary, or even tertiary, antibody or ligand can be used to detect the binding of a specific primary antibody. The membranes can then be stripped and reprobed with, for example, anti-β-actin antibody. The immunoreactive bands can then be subjected to densitometric analysis and the relative amounts of protein calculated by correction against the level of β actin within each sample.

High-throughput methods for detecting the presence or absence of proteins of interest or antibodies bound thereto are particularly preferred.

In some embodiments, MALDI-TOF is used for the rapid identification of a protein. Accordingly, there is no need to detect the proteins of interest using an antibody or ligand that specifically binds to the protein of interest. Rather, proteins from a sample are separated using gel electrophoresis using methods well known in the art and those proteins at approximately the correct molecular weight and/or isoelectric point are analysed using MALDI-TOF to determine the presence or absence of a protein of interest.

Alternatively, MALDI or ESI or a combination of approaches is used to determine the concentration of a particular protein in a sample, such as, for example PBMC.

Biosensor devices generally employ an electrode surface in combination with current or impedance measuring elements to be integrated into a device in combination with the assay substrate (such as that described in U.S. Pat. No. 5,567,301). An antibody or ligand that specifically binds to a protein of interest is preferably incorporated onto the surface of a biosensor device and a sample isolated from a subject is contacted to said device. A change in the detected current or impedance by the biosensor device indicates protein binding to said antibody or ligand. Some forms of biosensors known in the art also rely on surface plasmon resonance to detect protein interactions, whereby a change in the surface plasmon resonance surface of reflection is indicative of a protein binding to a ligand or antibody (U.S. Pat. Nos. 5,485,277 and 5,492,840).

Biosensors are of particular use in high throughput analysis due to the ease of adapting such systems to micro- or nano-scales. Furthermore, such systems are conveniently adapted to incorporate several detection reagents, allowing for multiplexing of diagnostic reagents in a single biosensor unit. This permits the simultaneous detection of several epitopes in a small amount of body fluids.

Evanescent biosensors are also preferred as they do not require the pretreatment of a sample prior to detection of a protein of interest. An evanescent biosensor generally relies upon light of a predetermined wavelength interacting with a fluorescent molecule, such as for example, a fluorescent antibody attached near the probe's surface, to emit fluorescence at a different wavelength upon binding of the diagnostic protein to the antibody or ligand.

To produce protein chips, the proteins, peptides, polypeptides, antibodies or ligands that are able to bind specific antibodies or proteins of interest are bound to a solid support such as for example glass, polycarbonate, polytetrafluoroethylene, polystyrene, silicon oxide and metal or silicon nitride. This immobilization is either direct (eg. by covalent linkage, such as, for example, Schiff's base formation, disulfide linkage, or amide or urea bond formation) or indirect. Methods of generating a protein chip are known in the art and are described in for example US Patent Application No. 20020136821, 20020192654, 20020102617 and U.S. Pat. No. 6,391,625. In order to bind a protein to a solid support it is often necessary to treat the solid support so as to create chemically reactive groups on the surface, such as, for example, with an aldehyde-containing silane reagent. Alternatively, an antibody or ligand may be captured on a microfabricated polyacrylamide gel pad and accelerated into the gel using microelectrophoresis as described in, Arenkov et al., 2000, Anal. Biochem., 278:123-131.

A protein chip is preferably generated such that several proteins, ligands or antibodies are arrayed on said chip. This format permits the simultaneous screening for the presence of several proteins in a sample.

Alternatively, a protein chip may comprise only one protein, ligand or antibody, and be used to screen one or more patient samples for the presence of one polypeptide of interest. Such a chip may also be used to simultaneously screen an array of samples for a specific protein of interest.

Preferably, a sample to be analysed using a protein chip is attached to a reporter molecule, such as, for example, a fluorescent molecule, a radioactive molecule, an enzyme, or an antibody that is detectable using methods well known in the art. Accordingly, by contacting a protein chip with a labelled sample and subsequent washing to remove any unbound proteins the presence of a bound protein is detected using methods well known in the art, such as, for example using a DNA microarray reader.

Alternatively, biomolecular interaction analysis-mass spectrometry (BIA-MS) is used to rapidly detect and characterise a protein present in complex biological samples at the low- to sub-fmole level (Nelson et al., 2000, Electrophoresis, 21: 1155-1163). One technique useful in the analysis of a protein chip is surface enhanced laser desorption/ionization-time of flight-mass spectrometry (SELDI-TOF-MS) technology to characterise a protein bound to the protein chip. Alternatively, the protein chip is analysed using ESI as described in US Patent Application 20020139751.

As will be apparent to the skilled artisan, protein chips are particularly amenable to multiplexing of detection reagents. Accordingly, several antibodies or ligands each able to specifically-bind a different peptide or protein may be bound to different regions of said protein chip. Analysis of a biological sample using said chip then permits the detecting of multiple proteins of interest.

In a further embodiment, the samples are analysed using ICAT, essentially as described in US Patent Application No. 20020076739. This system relies upon the labelling of a protein sample from one source (i.e. a healthy subject) with a reagent and the labelling of a protein sample from another source (i.e. an allergic subject) with a second reagent that is chemically identical to the first reagent, but differs in mass due to isotope composition. It is preferable that the first and second reagents also comprise a biotin molecule. Equal concentrations of the two samples are then mixed, and peptides recovered by avidin affinity chromatography. Samples are then analysed using mass spectrometry. Any difference in peak heights between the heavy and light peptide ions directly correlates with a difference in protein abundance in a sample. The identity of such proteins may then be determined using a method well known in the art, such as, for example MALDI-TOF, or ESI.

Microfluidic technology may also be used in the analysis of proteins (Figeys D, Gygi S P, McKinnon G, Aebersold R: An integrated microfluidics-tandem mass spectrometry system for automated protein analysis. Anal Chem 1998, 70:3728-3734; Figeys D, Aebersold R: High sensitivity analysis of proteins and peptides by capillary electrophoresis-tandem mass spectrometry: recent developments in technology and applications. Electrophoresis 1998, 19:885-892). For example, microfluidics can be linked with a mass spectrometric analysis of proteins or peptides. Thus, peptides can be adsorbed onto hydrophobic membranes, desalted, and through the use of microfluidics eluted in a controlled manner to allow the direct mass spectrometric analysis of picomole amounts of peptides by electrospray ionisation mass spectrometry procedures (Lion N, Gellon J O, Jensen H, Girault H H: On-chip protein sample desalting and preparation for direct coupling with electrospray ionization mass spectrometry. J Chromatogr A 2003, 1003:11-19). Combinatorial peptidomics (Soloviev M, Barry R, Scrivener E, Terrett J: Combinatorial peptidomics: a generic approach for protein expression profiling. J Nanobiotechnology 2003, 1:4) may also be used with integrated microfluidic systems.

As will be apparent to those skilled in the art a diagnostic or prognostic detection system as described herein may be a multiplexed assay. As used herein the term “multiplex”, shall be understood not only to mean the detection of two or more diagnostic or prognostic markers in a single sample simultaneously, but also to encompass consecutive detection of two or more diagnostic or prognostic markers in a single sample, simultaneous detection of two or more diagnostic or prognostic markers in distinct but matched samples, and consecutive detection of two or more diagnostic or prognostic markers in distinct but matched samples. As used herein the term “matched samples” shall be understood to mean two or more samples derived from the same initial sample, or two or more samples isolated at the same point in time.

Once the determination of the level of expression of a gene or protein of the invention has been achieved numerous applications are then available. These applications include, for example, predicting the development of an allergic disorder in a mammal, diagnosing an allergic disorder in a mammal, monitoring a mammal for progress of therapy for an allergic disorder, determining the potential responsiveness of a mammal suffering from a disorder to treatment for the disorder, predicting the risk of a mammal suffering from a disorder progressing to a more severe and/or persistent form of the allergic disorder, determining the immunological phenotype of an allergic disorder in a mammal, and identifying a mammal capable of responding to a specific immunotherapy.

In one aspect, the level of expression of a gene in a mammal is used to diagnose an allergic disorder in the mammal. This can be achieved by comparing the level of expression of the gene in a cell of the mammal with the level of expression of the gene of a non-allergic mammal of the same species, which cell has been contacted with the same allergen. If the level of expression of the two genes is different this is indicative that the test animal has an allergic disorder.

As used herein, the terms “diagnosis” or “diagnosing” refer to the method of distinguishing one allergic disorder from another allergic disorder, or determining whether an allergic disorder is present in an animal (atopic) relative to the “normal” or “non-allergic.” (non-atopic) state and/or determining the nature of an allergic disorder.

In another aspect the invention relates to a method for predicting the development of an allergic disorder in a mammal. The term “predicting the development” when used with reference to an allergic disorder means that the mammal does not have an allergic disorder or does not have clinical symptoms of an allergic disorder, but they have a propensity to develop an allergic disorder. As defined supra, terms “propensity” to develop an allergic disorder, “predisposition”, or “susceptibility”, or any similar phrase, means that an animal which can develop allergy has certain “allergy-associated genes” which are “activated” such that they are predictive of an animal's incidence of developing a particular disorder (e.g. asthma). The expression of these “allergy-associated genes” in mammals predisposed to an allergic disorder in comparison to non-allergic mammals is predictive of the development of an allergic disorder even in pre-symptomatic or pre-diseased mammals.

In some embodiments, the term “predicting the development” also includes mammals that have an allergic disorder and the methods disclosed herein are used to more accurately determine the severity of the disorder or predict its progression.

As described supra, the methods of the invention are capable of identifying subjects that have a pre-disposition or susceptibility to developing an allergic disease. Once mammalian subjects that are pre-disposed or susceptible to developing an allergic disease have been identified they can be treated and/or prevented from developing said allergy.

The terms “treatment,” “treating,” or “treat,” include the administration of a control agent (e.g. an agent capable of altering or effecting the relative amounts of proteins of interest) to a subject, who has an allergic disease or is at risk of suffering from an allergic disease, such that the allergic disease (or at least one symptom of the allergic disease) is cured, healed, prevented, alleviated, relieved, altered, remedied, ameliorated, improved or otherwise affected, preferably in an advantageous manner.

As used herein “prevention” means any prevention of an allergic disorder in a subject and includes preventing the disorder from occurring in an animal that has not yet been diagnosed as having it. The effect may be prophylactic in terms of completely or partially preventing the disorder or a sign or symptom thereof.

The language “effective amount” of a control agent is that amount necessary or sufficient to treat or prevent a particular allergic disease, e.g., to prevent the various morphological and somatic symptoms of the allergic disease. The effective amount can vary depending on such factors as the size and weight of the subject, the type of condition, or the particular agent. For example, the choice of the pharmaceutical composition can affect what constitutes an “effective amount.” One of ordinary skill in the art would be able to study the aforementioned factors and make the determination regarding the effective amount of the pharmaceutical composition without undue experimentation.

The control agents of the invention may be administered by any suitable route, and the person skilled in the art will readily be able to determine the most suitable route and dose for the allergic disease to be treated. Dosage will be at the discretion of the attendant physician or veterinarian, and will depend on the nature and state of the allergic disease to be treated, the age and general state of health of the subject to be treated, the route of administration, and any previous treatment which may have been administered.

Control agents useful in the present invention may be located by standard assays. Protocols for carrying out such assays are well known to those of skill in the art and need not be described in great detail here. The term “control agent” or “drug candidate” or “modulator” or “modifying agent” or grammatical equivalents as used herein describes any molecule, eg., protein, oligopeptide, small organic molecule, polysaccharide, polynucleotide, etc., to be tested for the capacity to directly or indirectly control the expression of the genes of interest e.g., a nucleic acid or protein sequence. In preferred embodiments, the control agents alter or modify the expression profiles of the nucleic acids shown in Table 1 or the proteins encoded by these nucleic acids. In some embodiments, the control agents will be capable of increasing the endogenous amount of particular proteins, while in other embodiments the control agents will merely supplement the endogenous amount of proteins.

In some embodiments, the control agents will reduce the endogenous amount of particular proteins.

The term “drug candidates” encompass numerous chemical classes, though typically they are organic molecules, preferably small organic compounds having a molecular weight of more than 100 and less than about 2,500 daltons. Preferred small molecules are less than 2000, or less than 1500 or less than 1000 or less than 500 Daltons. Candidate control agents comprise functional groups necessary for structural interaction with proteins, particularly hydrogen bonding, and typically include at least an amine, barbonyl, hydroxyl or carboxyl group, preferably at least two of the functional chemical groups. The candidate control agents often comprise cyclical carbon or heterocyclic structures and/or aromatic or polyaromatic structures substituted with one or more of the above functional groups. Candidate control agents are also found among biomolecules including peptides, saccharides, fatty acids, steroids, purines, pyrimidines, derivatives, structural analogs or combinations thereof. Particularly preferred are peptides.

Modulators of protein expression can also be nucleic acids, as defined below. As described above generally for proteins, nucleic acid modulating agents are naturally occurring nucleic acids, random nucleic acids, or “biased” random nucleic acids. For example, digests of prokaryotic or eukaryotic genomes are used as is outlined above for proteins.

In certain embodiments, the activity of a protein of interest is down-regulated, or entirely inhibited, by the use of antisense polynucleotide, i.e., a nucleic acid complementary to, and which can preferably hybridize specifically to, a coding mRNA nucleic acid sequence, e.g., protein, mRNA, or a subsequence thereof. Binding of the antisense polynucleotide to the mRNA reduces the translation and/or stability of the mRNA.

In the context of this invention, antisense nucleic acids can comprise naturally-occurring nucleotides, or synthetic species formed from naturally-occurring subunits or their close homologs. Antisense nucleic acids may also have altered sugar moieties or inter-sugar linkages. Exemplary among these are the phosphorothioate and other sulphur containing species which are known for use in the art. Analogs are comprehended by this invention so long as they function effectively to hybridize with the mRNA transcribed from the genes of interest. See, eg., Isis Pharmaceuticals, Carlsbad, Calif.; Sequitor, Inc., Natick, Mass.

Such antisense nucleic acids can readily be synthesized using recombinant means, or are synthesized in vitro. Equipment for such synthesis is sold by several vendors, including Applied Biosystems. The preparation of other oligonucleotides such as phosphorothioates and alkylated derivatives is also well known to those of skill in the art.

Antisense molecules as used herein include antisense or sense oligonucleotides. Sense oligonucleotides can, eg., be employed to block transcription by binding to the anti-sense strand. The antisense and sense oligonucleotide comprise a single-stranded nucleic acid sequence (either RNA or DNA) capable of binding to target mRNA (sense) or DNA (antisense) sequences. Antisense or sense oligonucleotides, according to the present invention, comprise a fragment generally at least about 14 nucleotides, preferably from about 14 to 30 nucleotides. The ability to derive an antisense or a sense oligonucleotide, based upon a cDNA sequence encoding a given protein is described in, eg., Stein & Cohen (Cancer Res. 48:2659 (1988 and van der Krol et al. 1988, Bio Techniques, 6:958).

In addition to antisense nucleic acids, ribozymes are used to target and inhibit transcription of nucleotide sequences. A ribozyme is an RNA molecule that catalytically cleaves other RNA molecules. Different kinds of ribozymes have been described, including group I ribozymes, hammerhead ribozymes, hairpin ribozymes, RNase P, and axhead ribozymes (see, eg., Castanotto et al., 1994, Adv. in Pharmacology, 25: 289-317) for a general review of the properties of different 5 ribozymes).

Methods of preparing ribozymes are well known to those of skill in the art (see, eg., WO94/26877; Ojwang et al., 1993, Proc. Natl. Acad. Sci. USA., 90:6340-6344; Yamada et al., 1994, Human Gene Therapy, 1:39-45; Leavitt et al., 1995, Proc. Natl. Acad. Sci. USA., 92:699-703; Leavitt et al., 1994, Human Gene Therapy, 5:1151-120; and Yamada et al., 1994, Virology, 205: 121-126).

Polynucleotide modulators of the genes of interest are introduced into a cell containing the target nucleotide sequence by formation of a conjugate with a ligand binding molecule, as described in WO91/04753. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or block entry of the sense or antisense oligonucleotide or its conjugated version into the cell. Alternatively, a polynucleotide modulator is introduced into a cell containing the target nucleic acid sequence, eg., by formation of a polynucleotide-lipid complex, as described in WO90/10448.

Gene expression monitoring is conveniently used to test candidate modulators (eg., protein, nucleic acid or small molecule). After the candidate control agent has been added and the cells allowed to incubate for some period of time, the sample containing a target sequence to be analysed is added to the biochip. If required, the target sequence is prepared using known techniques. For example, the sample are treated to lyse the cells, using known lysis buffers, electroporation, etc., with purification and/or amplification such as PCR performed as appropriate. For example, an in vitro transcription with labels covalently attached to the nucleotides is performed. Generally, the nucleic acids are labelled with biotin-FITC or PE, or with cy3 or cy5.

In a preferred embodiment, the target sequence is labelled with, eg., a fluorescent, a chemiluminescent, a chemical, or a radioactive signal, to provide a means of detecting the target sequence's specific binding to a probe. The label also are an enzyme, such as, alkaline phosphatase or horseradish peroxidase, which when provided with an appropriate substrate produces a product that are detected. Alternatively, the label is a labelled compound or small molecule, such as an enzyme inhibitor, that binds but is not catalyzed or altered by the enzyme. The label also is a moiety or compound, such as, an epitope tag or biotin which specifically binds to streptavidin. For the example of biotin, the streptavidin is labelled as described above, thereby, providing a detectable signal for the bound target sequence. Unbound labelled streptavidin is typically removed prior to analysis.

As will be appreciated by those in the art, these assays are direct hybridization assays or can comprise “sandwich assays”, which include the use of multiple probes, as is generally outlined in U.S. Pat. Nos. 5,681,702, 5,597,909, 5,545,730, 5,594,117, 5,591,584, 5,571,670, 5,580,731, 5,571,670, 5,591,584, 5,624,802, 5,635,352, 5,594,118, 5,359,100, 5,124,246 and 5,681,697, all of which are hereby incorporated by reference. In this embodiment, in general, the target nucleic acid is prepared as outlined above, and then added to the biochip comprising a plurality of nucleic acid probes, under conditions that allow the formation of a hybridization complex.

A variety of hybridization conditions are used in the present invention, including high, moderate and low stringency conditions as outlined above. The assays are generally run under stringency conditions which allow formation of the label probe hybridization complex only in the presence of target. Stringency is controlled by altering a step parameter that is a thermodynamic variable, including, but not limited to, temperature, formamide concentration, salt concentration, chaotropic salt concentration pH, organic solvent concentration, etc. These parameters may also be used to control non-specific binding, as is generally outlined in U.S. Pat. No. 5,681,697. Thus it is desirable to perform certain steps at higher stringency conditions to reduce non-specific binding.

The reactions outlined herein are accomplished in a variety of ways. Components of the reaction are added simultaneously, or sequentially, in different orders, with preferred embodiments outlined below. In addition, the reaction may include a variety of other reagents. These include salts, buffers, neutral proteins, e.g. albumin, detergents, etc. which are used to facilitate optimal hybridization and detection, and/or reduce non-specific or background interactions. Reagents that otherwise improve the efficiency of the assay, such as protease inhibitors, nuclease inhibitors, anti-microbial agents, etc., may also be used as appropriate, depending on the sample preparation methods and purity of the target.

The assay data are analysed to determine the expression levels, and changes in expression levels as between states, of individual genes, forming a gene expression profile.

Screens are performed to identify modulators of the genes of interest phenotype. In one embodiment, screening is performed to identify modulators that can induce or suppress a particular expression profile, thus preferably generating the associated phenotype. In another embodiment, eg., for diagnostic applications, having identified differentially expressed genes important in a particular state, screens are performed to identify modulators that alter expression of individual genes.

In addition screens are done for genes that are induced in response to a candidate agent. After identifying a modulator based upon its ability to suppress an expression pattern leading to a normal expression pattern, or to modulate a single gene expression profile so as to mimic the expression of the gene from normal tissue, a screen as described above are performed to identify genes that are specifically modulated in response to the agent.

Thus, in one embodiment, a test compound is administered to a population of cells know to express a particular pattern of gene expression. By “administration” or “contacting” herein is meant that the candidate control agent is added to the cells in such a manner as to allow the agent to act upon the cell, whether by uptake and intracellular action, or by action at the cell surface. In some embodiments, nucleic acid encoding a proteinaceous candidate agent (i.e., a peptide) are put into a viral construct such as an adenoviral or retroviral construct, and added to the cell, such that expression of the peptide agent is accomplished. Regulatable gene administration systems can also be used.

Once the test compound has been administered to the cells, the cells are washed if desired and are allowed to incubate under preferably physiological conditions for some period of time. The cells are then harvested and a new gene expression profile is generated, as outlined herein.

Assays to identify compounds with modulating activity are usually performed in vitro. For example, a polypeptide is first contacted with a potential modulator and incubated for a suitable amount of time, eg., from 0.5 to 48 hours. In one embodiment, the polypeptide levels are determined in vitro by measuring the level of protein or mRNA. The level of protein is measured using immunoassays such as western blotting, ELISA and the like with an antibody that selectively binds to the polypeptide or a fragment thereof. For measurement of mRNA, amplification, e.g., using PCR, LCR, or hybridization assays, e.g., northern hybridization, RNAse protection, dot blotting, are preferred. The level of protein or mRNA is detected using directly or indirectly labelled detection agents, eg., fluorescently or radioactively labelled nucleic acids, radioactively or enzymatically labelled antibodies, and the like, as described herein.

Alternatively, a reporter gene system can be devised using protein promoters operably linked to reporter genes such as luciferase, green fluorescent protein, CAT, or beta-gal. The reporter construct is typically transfected into a cell. After treatment with a potential modulator, the amount of reporter gene transcription, translation, or activity is measured according to standard techniques known to those of skill in the art.

Once initial candidate compounds or control agents are identified, variants are further screened to better evaluate structure activity relationships. In a preferred embodiment, binding assays are done. In general, purified or isolated gene product is used; that is, the gene products of one or more differentially expressed nucleic acids are made. For example, antibodies are generated to the protein gene products, and standard immunoassays are run to determine the amount of protein present.

Thus, in a preferred embodiment, the methods comprise combining a protein of interest and a candidate compound, and determining the binding of the compound to the protein.

Generally, in a preferred embodiment of the methods herein, a protein of interest or the candidate control agent is non-diffusably bound to an insoluble support having isolated sample receiving areas (e.g. a microliter plate, an array, etc.). The insoluble supports are made of any composition to which the compositions are bound, is readily separated from soluble material, and is otherwise compatible with the overall method of screening. The surface of such supports are solid or porous and of any convenient shape. Examples of suitable insoluble supports include microtitre plates, arrays, membranes and beads. These are typically made of glass, plastic (e.g., polystyrene), polysaccharides, nylon or nitrocellulose, Teflon™, etc. microtitre plates and arrays are especially convenient because a large number of assays are carried out simultaneously, using small amounts of reagents and samples. The particular manner of binding of the composition is not crucial so long as it is compatible with the reagents and overall methods of the invention, maintains the activity of the composition and is non-diffusable. Preferred methods of binding include the use of antibodies (which do not sterically block either the ligand binding site or activation sequence when the protein is bound to the support), direct binding to “sticky” or ionic supports, chemical cross-linking, the synthesis of the protein or agent-on the surface, etc. Following binding of the protein or agent, excess unbound material is removed by washing. The sample receiving areas may then be blocked through incubation with bovine serum albumin (BSA), casein or other innocuous protein or other moiety.

In a preferred embodiment, the protein of interest is bound to the support, and a test compound is added to the assay. Alternatively, the candidate agent is bound to the support and the protein is added. Novel binding agents include specific antibodies, non-natural binding agents identified in screens of chemical libraries, peptide analogs, etc. Of particular interest are screening assays for agents that have a low toxicity for human cells. A wide variety of assays are used for this purpose, including labelled in vitro protein-protein binding assays, electrophoretic mobility shift assays, immunoassays for protein binding, functional assays (phosphorylation assays, etc.) and the like.

The determination of the binding of the test modulating compound to the protein of interest is done in a number of ways. In a preferred embodiment, the compound is labelled, and binding determined directly, e.g., by attaching all or a portion of the protein to a solid support, adding a labelled candidate agent (e.g., a fluorescent label), washing off excess reagent, and determining whether the label is present on the solid support. Various blocking and washing steps are utilized as appropriate.

In some embodiments, only one of the components is labelled, e.g., the proteins (or proteinaceous candidate compounds) are labelled. Alternatively, more than one component is labelled with different labels, e.g., ¹²⁵I for the proteins and a fluorophore for the compound. Proximity reagents, e.g., quenching or energy transfer reagents are also useful.

In one embodiment, the binding of the test compound is determined by competitive binding assay. The competitor is a binding moiety known to bind to the target molecule (i.e., a protein of interest), such as an antibody, peptide, binding partner, ligand, etc. Under certain circumstances, there are competitive binding between the compound and the binding moiety, with the binding moiety displacing the compound. In one embodiment, the test compound is labelled. Either the compound, or the competitor, or both, is added first to the protein for a time sufficient to allow binding, if present. Incubations are performed at a temperature which facilitates optimal activity, typically between 4 and 40 C. Incubation periods are typically optimized, e.g., to facilitate rapid high throughput screening. Typically between 0.1 and 1 hour will be sufficient. Excess reagent is generally removed or washed away. The second component is then added, and the presence or absence of the labelled component is followed, to indicate binding.

In a preferred embodiment, the competitor is added first, followed by the test compound. Displacement of the competitor is an indication that the test compound is binding to the protein of interest and thus is capable of binding to, and potentially modulating, the activity of the protein. In this embodiment, either component is labelled. Thus, e.g., if the competitor is labelled, the presence of label in the wash solution indicates displacement by the agent. Alternatively, if the test compound is labelled, the presence of the label on the support indicates displacement.

In an alternative preferred embodiment, the test compound is added first, with incubation and washing, followed by the competitor. The absence, of binding by the competitor may indicate that the test compound is bound to the protein of interest with a higher affinity. Thus, if the test compound is labelled, the presence of the label on the support, coupled with a lack of competitor binding, may indicate that the test compound is capable of binding to the protein.

In a preferred embodiment, the methods comprise differential screening to identity agents that are capable of modulating the activity of the proteins. In this embodiment, the methods comprise combining a protein of interest and a competitor in a first sample. A second sample comprises a test compound, a protein of interest, and a competitor. The binding of the competitor is determined for both samples, and a change, or difference in binding between the two samples indicates the presence of an agent capable of binding to the protein of interest and potentially modulating its activity. That is, if the binding of the competitor is different in the second sample relative to the first sample, the agent is capable of binding to the protein of interest.

Alternatively, differential screening is used to identify drug candidates that bind to the native protein of interest, but cannot bind to modified protein. The structure of the protein is modelled, and used in rational drug design to synthesize agents that interact with that site. Drug candidates that affect the activity of a protein of interest is also identified by screening drugs for the ability to either enhance or reduce the activity of the protein.

Positive controls and negative controls are used in the assays. Preferably control and test samples are performed in at least triplicate to obtain statistically significant results. Incubation of all samples is for a time sufficient for the binding of the agent to the protein. Following incubation, samples are washed free of non-specifically bound material and the amount of bound, generally labelled agent determined. For example, where a radiolabel is employed, the samples are counted in a scintillation counter to determine the amount of bound compound.

A variety of other reagents are included in the screening assays. These include reagents like salts, neutral proteins, e.g. albumin, detergents, etc. which are used to facilitate optimal protein-protein binding and/or reduce non-specific or background interactions. Also reagents that otherwise improve the efficiency of the assay, such as protease inhibitors, nuclease inhibitors, anti-microbial agents, etc., are used. The mixture of components is added in an order that provides for the requisite binding.

In a preferred embodiment, the invention provides methods for screening for a compound capable of modulating the activity of a protein of interest. The methods comprise adding a test compound, as defined above, to a cell comprising test proteins. Preferred cell types include almost any cell. The cells contain a recombinant nucleic acid that encodes a protein of interest. In a preferred embodiment, a library of candidate agents is tested on a plurality of cells.

Kits for use in connection with the subject invention may also be provided. Such kits preferably include at least a set of known standards of the genes of interest or their encoded proteins and a set of probes that may, in certain kits, be present on the surface of an array, as discussed above. Kits may also contain instructions for using the kit to detect nucleic acid or protein using the methods described above.

The instructions are generally recorded on a suitable recording medium. For example, the instructions may be printed on a substrate, such as paper or plastic, etc. As such, the instructions may be present in the kits as a package insert, in the labelling of the container of the kit or components thereof (i.e., associated with the packaging or sub-packaging), etc. In other embodiments, the instructions are present as an electronic storage data file present on a suitable computer readable storage medium, e.g., CD-ROM, diskette, etc, including the same medium on which the program is presented.

In yet other embodiments, the instructions are not themselves present in the kit, but means for obtaining the instructions from a remote source, eg. via the Internet, are provided. An example of this embodiment is a kit that includes a web address where the instructions can be viewed from or from where the instructions can be downloaded.

Still further, the kit may be one in which the instructions are obtained are downloaded from a remote source, as in the Internet or World Wide Web. Some form of access security or identification protocol may be used to limit access to those entitled to use the subject invention. As with the instructions, the means for obtaining the instructions and/or programming is generally recorded on a suitable recording medium.

Appropriate control agents might also be formulated for administration. For example, carriers, diluents and other excipients can be admixed with the control agents to enable administration. The type of carrier, diluent or excipient will depend on the route of administration, and again the person skilled in the art will readily be able to determine the most suitable formulation for each particular case.

Methods and pharmaceutical carriers for preparation of pharmaceutical compositions or control agents are well known in the art, as set out in textbooks such as Remington's Pharmaceutical Sciences, 20th Edition, Williams & Wilkins, Pennsylvania, USA.

In the manufacture of control agents according to embodiments of the invention, a control agent of the invention is typically admixed with, inter alia, a pharmaceutically acceptable carrier. The carrier must, of course, be acceptable in the sense of being compatible with any other ingredients in the pharmaceutical composition and should not be deleterious to the mammal being treated. The carrier may be a solid or a liquid, or both, and is preferably formulated with the molecule of the invention as a unit-dose formulation, for example, a tablet, which may contain from about 0.01 or 0.5% to about 95% or 99% by weight of the molecule. The pharmaceutical compositions may be prepared by any of the well-known techniques of pharmacy including, but not limited to, admixing the components, optionally including one or more accessory ingredients.

Pharmaceutical compositions and/or control agents suitable for oral administration may be presented in discrete units, such as capsules, cachets, lozenges, or tablets, each containing a predetermined amount of the agent of the invention; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the agent and a suitable carrier (which may contain one or more accessory ingredients as noted above). In general, a pharmaceutical composition according to embodiments of the invention is prepared by uniformly and intimately admixing the agent of the invention with a liquid or finely divided solid carrier, or both, and then, if necessary, shaping the resulting mixture. For example, a tablet may be prepared by compressing or moulding a powder or granules containing the mixture of the agent and pharmaceutically acceptable carrier, optionally with one or more accessory ingredients. Compressed tablets may be prepared by compressing, in a suitable machine, the mixture in a free-flowing form, such as a powder or granules optionally mixed with a binder, lubricant, inert diluent, and/or surface active/dispersing agent(s). Moulded tablets may be made by moulding, in a suitable machine, the powdered compound moistened with an inert liquid binder.

Pharmaceutical compositions suitable for buccal (sub-lingual) administration include lozenges comprising a agent of the invention in a flavoured base, usually sucrose and acacia or tragacanth; and pastilles comprising the agent of the invention in an inert base such as gelatin and glycerin or sucrose and acacia.

Pharmaceutical compositions according to some embodiments of the invention are suitable for parenteral administration and comprise sterile aqueous and non-aqueous injection solutions of a agent of the invention, which preparations are preferably isotonic with the blood of the intended recipient. These preparations may contain anti-oxidants, buffers, bacteriostats and solutes which render the composition isotonic with the blood of the intended recipient. Aqueous and non-aqueous sterile suspensions may include suspending agents and thickening agents. The compositions may be presented in unit/dose or multi-dose containers, for example sealed ampoules and vials, and may be stored in a freeze-dried (lyophilised) condition requiring only the addition of the sterile liquid carrier, for example, saline or water-for-injection immediately prior to use. Extemporaneous injection solutions and suspensions may be prepared from sterile powders, granules and tablets of the kind previously described. For example, an injectable, stable, sterile composition comprising an agent of the invention in a unit dosage form in a sealed container may be provided. The unit dosage form typically comprises from about 10 mg to about 10 grams of the agent of the invention. When the agent is substantially water-insoluble, a sufficient amount of emulsifying agent, which is physiologically acceptable may be employed in sufficient quantity to emulsify the agent in an aqueous carrier. One such useful emulsifying agent is phosphatidyl choline.

Pharmaceutical compositions suitable for rectal administration are preferably presented as unit dose suppositories. These may be prepared by admixing a agent of the invention with one or more conventional solid carriers, for example, cocoa butter, and then shaping the resulting mixture.

Pharmaceutical compositions suitable for topical application to the skin preferably take the form of an ointment, cream, lotion, paste, gel, spray, aerosol, or oil. Carriers which may be used include petroleum jelly, lanoline, polyethylene glycols, alcohols, and transdermal enhancers.

Pharmaceutical compositions suitable for transdermal administration may be presented as discrete patches adapted to remain in intimate contact with the epidermis of the recipient for a prolonged period of time. Compositions suitable for transdermal administration may also be delivered by iontophoresis (see, for example, Pharmaceutical Research 3 (6):318 (1986)) and typically take the form of an optionally buffered aqueous solution of an agent of the invention. Suitable formulations comprise citrate or bis/tris buffer (pH 6) or ethanol/water and contain from 0.1 to 0.2 M active ingredient.

In addition to any of the ingredients listed above, the composition may further comprise other agents. For example, agents such as binders, sweeteners, thickeners, flavouring agents, disintegrating agents, coating agents, preservatives, lubricants, and/or time delay agents.

As mentioned above, the control agents of the invention are associated with allergic disorders and hence an agent of the invention, and compositions comprising a control agent of the invention, may be used in the treatment or prevention of an allergic disorder.

In order to use a control agent of the invention in the treatment or prevention of an allergic disorder, the agent must be administered to a mammal.

An agent of the invention may be administered to the mammal by any suitable route, and the person skilled in the art will readily be able to determine the most suitable route and dose for the condition to be treated. Dosage will be at the discretion of the attendant physician or veterinarian, and will depend on the route of administration, the nature and state of the condition to be treated, the age and general state of health of the subject to be treated, and any previous treatment which may have been administered.

An agent of the invention may be administered to the mammal periodically or repeatedly and may be administered by one or more of the following routes: oral, rectal, topical, inhalation (eg., via an aerosol) buccal (eg., sub-lingual), vaginal, parenteral (eg., subcutaneous, intramuscular, intradermal, intraarticular, intrapleural, intraperitoneal, intracerebral, intraarterial, or intravenous), topical (ie., both skin and mucosal surfaces, including airway surfaces) and transdermal administration. The most suitable route in any given case will depend on the nature and severity of the condition being treated and on the nature of the particular molecule of the invention which is administered.

In some embodiments an allergic disorder may be treated or prevented by administering an agent capable of modulating the expression of a nucleic acid molecule of the invention or which specifically binds to a polypeptide of the invention.

Another method of treating or preventing an allergic disorder in a mammal is to administer to the mammal an agent which specifically binds to a polypeptide encoded by a nucleic acid molecule of the invention.

An antibody that “specifically binds to” or is “specific for” a particular polypeptide or an epitope on a particular polypeptide is one that binds to that particular polypeptide or epitope on a particular polypeptide without substantially binding to any other polypeptide or polypeptide epitope.

The term “antibody” is used in the broadest sense and includes fragments of antibodies which specifically bind to a particular polypeptide or an epitope on a particular polypeptide. The term “monoclonal antibody” as used herein refers to an antibody obtained from a population of substantially homogeneous antibodies, i.e., the individual antibodies comprising the population are identical except for possible naturally-occurring mutations that may be present in minor amounts.

“Antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies (Zapata et al., Protein Eng., 8(10):1057-1062 [1995]); single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen-combining sites and is still capable of cross-linking antigen.

“Fv” is the minimum antibody fragment which contains a complete antigen-recognition and binding site. This region consists of a dimer of one heavy- and one light-chain variable domain in tight, non-covalent association. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (CH1) of the heavy chain. Fab fragments differ from Fab′ fragments by the addition of a few residues at the carboxy terminus of the heavy chain CH 1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains.

Depending on the amino acid sequence of the constant domain of their heavy chains, immununoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2.

“Single-chain Fv” or “sFv” antibody fragments comprise the V_{H} and V_{L} domains of antibody, wherein these domains are present in a single polypeptide chain. Preferably, the Fv polypeptide further comprises a polypeptide linker between the V_{H} and V_{L} domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example. EP 404,097; WO 93/11161: and Hollinger et al., Proc. Natl. Acad. Sci. USA, 90:6444-6448 (1993).

By “comprising” is meant including, but not limited to, whatever follows the word comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The following examples, which describe exemplary techniques and experimental results, are provided for the purpose of illustrating the invention, and should not be construed as limiting.

### Example 1

### Use of Microarray Analysis to Determine Specific expression of mRNA in Allergic and Non-Allergic Subjects in Response to Allergen

Blood samples were obtained from allergic individuals, who were selected on the basis of positive skin prick test reactivity to House Dust Mite (HDM), together with samples from non-allergic controls who were tested for the presence of HDM-specific IgE in serum and were all negative. The presence of IgE to HDM was defined by the EAST (CAP) system (Pharmacia, Australia), and the allergic volunteers in this study displayed RAST (CAP) scores ≧2.

Freshly isolated peripheral blood mononuclear cells (PBMC) were resuspended at 1×10⁶ cells/ml and 1 ml of the cell suspension was cultured for 6, 12, 24, or 48 hours at 37° C., 5 CO₂ in round bottom tubes or multi-well plates in serum-free medium AIM-V⁴ (Life Technologies, Mulgrave, Australia) supplemented with 4×10⁻⁵ 2-mercaptoethanol, with or without the addition of 10 μg/ml of whole extract of HDM (Dermatophagoides pteronyssinus, CSL Limited, Parkville, Australia);

At each time point, equal sized aliquots of cells were centrifuged and the cell pellets were used immediately for total RNA extraction. Alternatively, Dynabeads™ were used to positively select CD8 T cells followed by CD4 T cells and then RNA was extracted. Extraction of the RNA was by performed by standard techniques. Total RNA was extracted using TRIZOL (Invitrogen) followed by an RNAeasy minikit (QIAGEN).

The extracted RNA was pooled from the individuals in each group (allergic and non-allergic) and then labelled and hybridised to Affymetrix™ U133a or U133plus2 arrays using the standard Affymetrix™ protocols (http://www.affymetrix.com/index.affx). Samples of the individual RNAs in the pools were kept separate for subsequent quantitative RT-PCR validation studies (see Example 2 below).

Data from these microarray experiments are shown in Tables 2 to 8 as fluorescent microarray units (stimulated vs unstimulated cultures).

Table 2 shows a list of the gene expression levels in HDM-sensitive atopic (+) and non-HDM-sensitive atopic (−) CD4 T-cells purified from peripheral blood mononuclear cells which were cultured in the absence (C) or presence of house dust mite allergen (HDM) for 24 hours. Gene expression levels are expressed in fluorescent microarray units. Values in bold italics indicate where a difference was observed in atopic and non-atopic gene expression patterns.

Table 3 shows a list of the gene expression levels in HDM-sensitive atopic (+) and non-HDM-sensitive atopic (−) CD8 T-cells purified from peripheral blood mononuclear cells which were cultured in the absence (C) or presence of house dust mite allergen (HDM) for 24 hours. Gene expression levels are expressed in fluorescent microarray units. Values in bold italics indicate where a difference was observed in atopic and non-atopic gene expression patterns.

Table 4 shows a list of the gene expression levels in HDM-sensitive atopic (+) and non-HDM-sensitive atopic (−) T-cell depleted peripheral blood mononuclear cells which were cultured in the absence (C) or presence of house dust mite allergen (HDM) for 24 hours. Gene expression levels are expressed in fluorescent microarray units. Values in bold italics indicate where a difference was observed in atopic and non-atopic gene expression patterns.

Table 5 shows a list of the gene expression levels in HDM-sensitive atopic (+) and non-HDM-sensitive atopic (−) peripheral blood mononuclear cells which were cultured in the absence (C) or presence of house dust mite allergen (HDM) for 6 hours. Gene expression levels are expressed in fluorescent microarray units. Values in bold italics indicate where a difference was observed in atopic and non-topic gene expression patterns.

Table 6 shows a list of the gene expression levels in HDM-sensitive atopic (+) and non-HDM-sensitive atopic (−) peripheral blood mononuclear cells which were cultured in the absence (C) or presence of house dust mite allergen (HDM) for 12 hours. Gene expression levels are expressed in fluorescent microarray units. Values in bold italics indicate where a difference was observed in atopic and non-atopic gene expression patterns.

Table 7 shows a list of the gene expression levels in HDM-sensitive atopic (+) and non-HDM-sensitive atopic (−) peripheral blood mononuclear cells which were cultured in the absence (C) or presence of house dust mite allergen (HDM) for 24 hours. Gene expression levels are expressed in fluorescent microarray units. Values in bold italics indicate where a difference was observed in atopic and non-atopic gene expression patterns.

Table 8 shows a list of the gene expression levels in HDM-sensitive atopic (+) and non-HDM-sensitive atopic (−) peripheral blood mononuclear cells which were cultured in the absence (C) or presence of house dust mite allergen (HDM) for 48 hours. Gene expression levels are expressed in fluorescent microarray units. Values in bold italics indicate where a difference was observed in atopic and non-atopic gene expression patterns.

Data were analysed with the rma algorithm using the statistical package R (Irizarry R. A. et al. 2003, Biostatistics 4(2):249-64). Genes were considered differentially expressed (between stimulated and unstimulated cultures) if the fold-change value was greater than the cut-off value (background noise). Cut-off values were determined based on the standard deviation of the noise for each experiment. Genes with large fold-change values between allergic individuals and non-allergic individuals were then identified.

Interpretation of these data is as follows: expression of genes that are indicative of allergic disorder are those in which the figure for atopic (allergy suffers) are higher than the figure for non-allergic individuals (non-atopic individuals). For example, as shown in Table 7, MELK is expressed at higher levels in allergic subjects who were exposed to house dust mite (HDM) allergen for 48 hours compared with the levels in non-atopic subjects.

TABLE 2

| | Atopic status | | | | | | | |
|---|---|---|---|---|---|---|---|---|
| | + | + | + | + | − | − | − | − |
| | Stimulus | | | | | | | |
| | C | HDM | C | HDM | C | HDM | C | HDM |
| CAMK2D | 23 | 81 | 24 | 67 | 29 | 28 | 29 | 18 |
| CDH1 | 8 | 25 | 8 | 29 | 6 | 9 | 8 | 11 |
| SLC37A3 | 24 | 68 | 26 | 78 | 29 | 32 | 24 | 25 |
| PALM2- | 106 | 266 | 79 | 165 | 135 | 155 | 120 | 91 |
| AKAP2 | | | | | | | | |
| NSMCE1 | 222 | 503 | 240 | 572 | 231 | 233 | 225 | 213 |
| TSPAN13 | 10 | 44 | 9 | 48 | 14 | 15 | 10 | 15 |
| SYTL3 | 14 | 38 | 26 | 30 | 19 | 12 | 25 | 20 |
| SFRS8 | 130 | 131 | 122 | 123 | 122 | 118 | 116 | 116 |
| FIP1L1 | 79 | 103 | 92 | 86 | 114 | 113 | 97 | 89 |
| MAML3 | 9 | 8 | 10 | 6 | 15 | 8 | 9 | 9 |
| TRIM4 | 82 | 81 | 84 | 64 | 82 | 76 | 60 | 65 |
| SIAH1 | 176 | 171 | 192 | 151 | 156 | 130 | 144 | 130 |
| ITPR1 | 24 | 23 | 29 | 19 | 28 | 20 | 16 | 18 |
| ITSN2 | 44 | 46 | 43 | 39 | 46 | 49 | 43 | 48 |
| CLCF1 | 13 | 10 | 11 | 11 | 9 | 9 | 8 | 10 |
| CRLF1 | 15 | 13 | 12 | 12 | 13 | 16 | 18 | 13 |
| CLIC5 | 22 | 16 | 20 | 14 | 20 | 21 | 27 | 18 |
| IGJ | 5 | 39 | 4 | 60 | 9 | 14 | 5 | 11 |
| NFKBIZ | 321 | 850 | 306 | 671 | 310 | 417 | 377 | 360 |
| DLC1 | 5 | 12 | 6 | 20 | 6 | 8 | 6 | 7 |
| GBP5 | 48 | 122 | 51 | 112 | 68 | 104 | 72 | 86 |
| PEG10 | 5 | 6 | 7 | 5 | 5 | 5 | 5 | 5 |
| HOMER2 | 9 | 13 | 9 | 13 | 10 | 12 | 9 | 11 |
| ZBTB8 | 5 | 6 | 5 | 6 | 5 | 6 | 6 | 5 |
| MOBKL2C | 28 | 28 | 34 | 25 | 38 | 36 | 24 | 41 |
| EDG3 | 8 | 7 | 6 | 8 | 7 | 6 | 9 | 10 |
| MELK | 9 | 10 | 8 | 9 | 8 | 9 | 9 | 11 |
| PHC3 | 15 | 17 | 15 | 14 | 19 | 18 | 13 | 15 |
| TTC3 | 81 | 100 | 101 | 106 | 89 | 95 | 89 | 93 |

TABLE 3

| | Atopic status | | | | | | | |
|---|---|---|---|---|---|---|---|---|
| | + | + | + | + | − | − | − | − |
| | Stimulus | | | | | | | |
| | C | HDM | C | HDM | C | HDM | C | HDM |
| CAMK2D | 25 | 28 | 22 | 53 | 22 | 23 | 26 | 20 |
| CDH1 | 8 | 10 | 7 | 8 | 7 | 7 | 8 | 6 |
| SLC37A3 | 23 | 34 | 22 | 59 | 22 | 23 | 22 | 23 |
| PALM2- | 82 | 133 | 63 | 93 | 69 | 68 | 64 | 61 |
| AKAP2 | | | | | | | | |
| NSMCE1 | 205 | 252 | 226 | 327 | 215 | 208 | 202 | 197 |
| TSPAN13 | 17 | 15 | 10 | 13 | 14 | 13 | 14 | 13 |
| SYTL3 | 15 | 15 | 30 | 20 | 14 | 13 | 25 | 16 |
| SFRS8 | 119 | 125 | 122 | 122 | 114 | 116 | 114 | 120 |
| FIP1L1 | 92 | 104 | 102 | 90 | 99 | 100 | 100 | 100 |
| MAML3 | 14 | 9 | 12 | 11 | 14 | 12 | 22 | 12 |
| TRIM4 | 84 | 77 | 76 | 69 | 51 | 50 | 67 | 48 |
| SIAH1 | 140 | 137 | 168 | 163 | 128 | 118 | 120 | 126 |
| ITPR1 | 34 | 24 | 31 | 32 | 26 | 27 | 33 | 27 |
| ITSN2 | 50 | 51 | 59 | 50 | 56 | 55 | 48 | 54 |
| CLCF1 | 11 | 9 | 12 | 13 | 10 | 9 | 10 | 11 |
| CRLF1 | 13 | 16 | 11 | 17 | 14 | 15 | 15 | 13 |
| CLIC5 | 15 | 21 | 21 | 21 | 22 | 22 | 24 | 16 |
| IGJ | 9 | 13 | 7 | 10 | 13 | 13 | 9 | 10 |
| NFKBIZ | 302 | 475 | 313 | 484 | 220 | 273 | 216 | 240 |
| DLC1 | 7 | 11 | 7 | 33 | 7 | 7 | 6 | 6 |
| GBP5 | 79 | 173 | 63 | 137 | 91 | 79 | 82 | 98 |
| PEG10 | 8 | 5 | 5 | 6 | 7 | 8 | 6 | 5 |
| HOMER2 | 10 | 18 | 9 | 14 | 8 | 12 | 10 | 10 |
| ZBTB8 | 7 | 10 | 7 | 8 | 7 | 6 | 8 | 6 |
| MOBKL2C | 42 | 44 | 37 | 43 | 46 | 44 | 32 | 33 |
| EDG3 | 8 | 10 | 8 | 9 | 9 | 8 | 8 | 7 |
| MELK | 8 | 9 | 6 | 10 | 10 | 10 | 9 | 7 |
| PHC3 | 17 | 15 | 17 | 15 | 18 | 17 | 15 | 14 |
| TTC3 | 90 | 90 | 72 | 88 | 101 | 105 | 99 | 84 |

TABLE 4

| | Atopic status | | | | | | | |
|---|---|---|---|---|---|---|---|---|
| | + | + | + | + | − | − | − | − |
| | Stimulus | | | | | | | |
| | C | HDM | C | HDM | C | HDM | C | HDM |
| CAMK2D | 39 | 35 | 35 | 39 | 40 | 30 | 42 | 30 |
| CDH1 | 8 | 9 | 7 | 9 | 6 | 8 | 5 | 7 |
| SLC37A3 | 9 | 16 | 11 | 15 | 11 | 11 | 11 | 10 |
| PALM2- | 490 | 904 | 406 | 978 | 513 | 471 | 598 | 484 |
| AKAP2 | | | | | | | | |
| NSMCE1 | 205 | 249 | 210 | 244 | 210 | 205 | 195 | 197 |
| TSPAN13 | 100 | 116 | 110 | 116 | 94 | 81 | 72 | 75 |
| SYTL3 | 10 | 12 | 13 | 13 | 12 | 11 | 16 | 12 |
| SFRS8 | 88 | 93 | 90 | 82 | 87 | 85 | 86 | 100 |
| FIP1L1 | 111 | 105 | 97 | 112 | 101 | 100 | 102 | 95 |
| MAML3 | 13 | 16 | 20 | 17 | 26 | 13 | 22 | 19 |
| TRIM4 | 54 | 57 | 58 | 52 | 66 | 51 | 56 | 47 |
| SIAH1 | 73 | 67 | 79 | 65 | 81 | 72 | 74 | 70 |
| ITPR1 | 30 | 32 | 36 | 34 | 44 | 28 | 39 | 29 |
| ITSN2 | 29 | 36 | 35 | 43 | 36 | 31 | 34 | 33 |
| CLCF1 | 14 | 13 | 15 | 15 | 13 | 16 | 14 | 19 |
| CRLF1 | 21 | 23 | 17 | 22 | 15 | 14 | 17 | 17 |
| CLIC5 | 19 | 16 | 16 | 18 | 15 | 16 | 19 | 19 |
| IGJ | 64 | 133 | 50 | 146 | 93 | 104 | 50 | 83 |
| NFKBIZ | 264 | 400 | 241 | 389 | 267 | 317 | 308 | 305 |
| DLC1 | 11 | 9 | 10 | 11 | 10 | 11 | 9 | 7 |
| GBP5 | 89 | 84 | 76 | 90 | 85 | 60 | 89 | 91 |
| PEG10 | 18 | 57 | 22 | 98 | 22 | 14 | 14 | 15 |
| HOMER2 | 21 | 66 | 21 | 84 | 18 | 24 | 20 | 20 |
| ZBTB8 | 9 | 32 | 10 | 37 | 13 | 9 | 10 | 9 |
| MOBKL2C | 37 | 114 | 34 | 119 | 43 | 53 | 48 | 45 |
| EDG3 | 11 | 11 | 10 | 10 | 9 | 13 | 11 | 10 |
| MELK | 9 | 9 | 9 | 10 | 13 | 9 | 9 | 10 |
| PHC3 | 12 | 17 | 15 | 16 | 19 | 17 | 16 | 16 |
| TTC3 | 66 | 65 | 65 | 48 | 71 | 62 | 70 | 58 |

TABLE 5

| | Atopic status | | | | | | | |
|---|---|---|---|---|---|---|---|---|
| | + | + | + | + | − | − | − | − |
| | Stimulus | | | | | | | |
| | C | HDM | C | HDM | C | HDM | C | HDM |
| CAMK2D | 60 | 64 | 52 | 39 | 62 | 44 | 53 | 44 |
| CDH1 | 37 | 44 | 24 | 24 | 18 | 27 | 22 | 41 |
| SLC37A3 | 25 | 28 | 23 | 31 | 29 | 21 | 23 | 25 |
| PALM2- | 335 | 816 | 386 | 857 | 367 | 567 | 534 | 572 |
| AKAP2 | | | | | | | | |
| NSMCE1 | 260 | 303 | 260 | 295 | 260 | 264 | 279 | 278 |
| TSPAN13 | 128 | 251 | 179 | 298 | 153 | 271 | 149 | 271 |
| SYTL3 | 45 | 53 | 37 | 37 | 33 | 39 | 24 | 45 |
| SFRS8 | 64 | 171 | 72 | 98 | 115 | 68 | 144 | 52 |
| FIP1L1 | 120 | 168 | 54 | 111 | 136 | 94 | 174 | 115 |
| MAML3 | 36 | 32 | 30 | 35 | 81 | 37 | 73 | 25 |
| TRIM4 | 69 | 123 | 84 | 103 | 95 | 69 | 126 | 78 |
| SIAH1 | 89 | 128 | 67 | 108 | 105 | 87 | 108 | 101 |
| ITPR1 | 20 | 21 | 13 | 23 | 22 | 16 | 23 | 15 |
| ITSN2 | 40 | 51 | 50 | 44 | 34 | 48 | 35 | 38 |
| CLCF1 | 26 | 18 | 22 | 25 | 19 | 18 | 19 | 21 |
| CRLF1 | 29 | 29 | 27 | 35 | 41 | 32 | 30 | 33 |
| CLIC5 | 34 | 36 | 37 | 37 | 35 | 38 | 30 | 44 |
| IGJ | 124 | 144 | 134 | 181 | 85 | 112 | 88 | 86 |
| NFKBIZ | 733 | 2160 | 906 | 1920 | 672 | 1800 | 740 | 1745 |
| DLC1 | 12 | 15 | 15 | 15 | 15 | 16 | 13 | 16 |
| GBP5 | 140 | 149 | 220 | 127 | 222 | 105 | 383 | 145 |
| PEG10 | 14 | 32 | 26 | 90 | 26 | 19 | 21 | 24 |
| HOMER2 | 18 | 21 | 22 | 25 | 26 | 20 | 27 | 16 |
| ZBTB8 | 13 | 13 | 14 | 17 | 17 | 15 | 11 | 17 |
| MOBKL2C | 73 | 83 | 124 | 117 | 71 | 59 | 100 | 89 |
| EDG3 | 20 | 23 | 23 | 28 | 23 | 25 | 15 | 21 |
| MELK | 18 | 16 | 19 | 17 | 15 | 16 | 17 | 22 |
| PHC3 | 41 | 57 | 35 | 40 | 41 | 36 | 39 | 35 |
| TTC3 | 85 | 95 | 77 | 52 | 76 | 70 | 91 | 56 |

TABLE 6

| | Atopic status | | | | | | | | | | | |
|---|---|---|---|---|---|---|---|---|---|---|---|---|
| | + | + | + | + | + | + | − | − | − | − | − | − |
| | Stimulus | | | | | | | | | | | |
| | C | HDM | C | HDM | C | HDM | C | HDM | C | HDM | C | HDM |
| CAMK2D | 47 | 73 | 63 | 68 | 45 | 57 | 54 | 62 | 63 | 56 | 39 | 45 |
| CDH1 | 17 | 28 | 14 | 24 | 15 | 24 | 15 | 18 | 13 | 22 | 14 | 21 |
| SLC37A3 | 24 | 38 | 22 | 41 | 19 | 29 | 16 | 24 | 24 | 27 | 19 | 23 |
| PALM2-AKAP2 | 301 | 720 | 603 | 1026 | 352 | 776 | 354 | 507 | 406 | 605 | 361 | 543 |
| NSMCE1 | 293 | 429 | 286 | 445 | 259 | 363 | 290 | 270 | 261 | 313 | 287 | 252 |
| TSPAN13 | 102 | 114 | 151 | 183 | 103 | 114 | 110 | 153 | 98 | 133 | 98 | 114 |
| SYTL3 | 35 | 68 | 23 | 38 | 23 | 23 | 32 | 25 | 30 | 30 | 25 | 23 |
| SFRS8 | 74 | 89 | 115 | 94 | 158 | 157 | 48 | 30 | 175 | 37 | 128 | 152 |
| FIP1L1 | 136 | 175 | 73 | 94 | 190 | 153 | 174 | 76 | 131 | 83 | 155 | 202 |
| MAML3 | 24 | 21 | 26 | 20 | 16 | 14 | 27 | 18 | 34 | 22 | 12 | 14 |
| TRIM4 | 100 | 77 | 89 | 91 | 69 | 79 | 89 | 72 | 90 | 72 | 71 | 70 |
| SIAH1 | 124 | 123 | 113 | 122 | 116 | 120 | 144 | 133 | 149 | 133 | 140 | 118 |
| ITPR1 | 21 | 28 | 24 | 21 | 15 | 14 | 17 | 19 | 23 | 23 | 18 | 16 |
| ITSN2 | 32 | 70 | 38 | 49 | 37 | 46 | 46 | 38 | 49 | 46 | 45 | 43 |
| CLCF1 | 19 | 23 | 20 | 23 | 23 | 28 | 24 | 16 | 23 | 19 | 31 | 23 |
| CRLF1 | 29 | 33 | 27 | 40 | 27 | 33 | 33 | 31 | 33 | 31 | 35 | 26 |
| CLIC5 | 22 | 30 | 36 | 26 | 38 | 39 | 35 | 33 | 27 | 35 | 36 | 33 |
| IGJ | 95 | 163 | 136 | 231 | 87 | 147 | 77 | 83 | 78 | 123 | 88 | 166 |
| NFKBIZ | 559 | 1402 | 823 | 1586 | 544 | 1072 | 648 | 1176 | 633 | 1148 | 422 | 963 |
| DLC1 | 14 | 15 | 14 | 19 | 13 | 19 | 15 | 20 | 14 | 14 | 16 | 12 |
| GBP5 | 100 | 105 | 99 | 110 | 244 | 219 | 129 | 131 | 112 | 121 | 176 | 248 |
| PEG10 | 18 | 37 | 30 | 81 | 17 | 18 | 19 | 18 | 18 | 18 | 16 | 14 |
| HOMER2 | 21 | 22 | 18 | 24 | 24 | 26 | 21 | 16 | 21 | 17 | 23 | 27 |
| ZBTB8 | 12 | 15 | 14 | 24 | 13 | 14 | 15 | 14 | 17 | 15 | 13 | 11 |
| MOBKL2C | 102 | 179 | 115 | 124 | 167 | 209 | 179 | 114 | 146 | 111 | 172 | 143 |
| EDG3 | 24 | 32 | 17 | 28 | 30 | 34 | 18 | 35 | 16 | 32 | 23 | 34 |
| MELK | 21 | 15 | 19 | 17 | 19 | 17 | 15 | 20 | 19 | 14 | 14 | 15 |
| PHC3 | 28 | 29 | 30 | 40 | 35 | 35 | 25 | 26 | 37 | 22 | 27 | 34 |
| TTC3 | 77 | 74 | 55 | 51 | 101 | 76 | 97 | 72 | 56 | 44 | 101 | 92 |

TABLE 7

| | Atopic status | | | | | | | | | | | |
|---|---|---|---|---|---|---|---|---|---|---|---|---|
| | + | + | + | + | + | + | − | − | − | − | − | − |
| | Stimulus | | | | | | | | | | | |
| | C | HDM | C | HDM | C | HDM | C | HDM | C | HDM | C | HDM |
| CAMK2D | 50 | 71 | 53 | 43 | 48 | 65 | 62 | 56 | 58 | 64 | 54 | 36 |
| CDH1 | 15 | 26 | 12 | 19 | 12 | 32 | 15 | 17 | 13 | 18 | 14 | 23 |
| SLC37A3 | 24 | 40 | 22 | 25 | 20 | 37 | 22 | 23 | 21 | 28 | 18 | 23 |
| PALM2-AKAP2 | 300 | 736 | 408 | 805 | 417 | 668 | 377 | 407 | 360 | 414 | 334 | 402 |
| NSMCE1 | 397 | 565 | 340 | 422 | 284 | 409 | 387 | 373 | 318 | 329 | 310 | 294 |
| TSPAN13 | 113 | 108 | 120 | 157 | 96 | 138 | 96 | 126 | 105 | 133 | 106 | 130 |
| SYTL3 | 24 | 22 | 19 | 16 | 24 | 24 | 19 | 22 | 25 | 27 | 25 | 23 |
| SFRS8 | 67 | 72 | 106 | 101 | 132 | 128 | 63 | 90 | 97 | 51 | 144 | 116 |
| FIP1L1 | 141 | 98 | 124 | 29 | 141 | 168 | 121 | 88 | 168 | 35 | 154 | 147 |
| MAML3 | 23 | 15 | 28 | 16 | 14 | 11 | 17 | 14 | 30 | 15 | 15 | 12 |
| TRIM4 | 79 | 67 | 54 | 58 | 73 | 77 | 75 | 49 | 73 | 60 | 54 | 45 |
| SIAH1 | 182 | 186 | 122 | 108 | 143 | 125 | 166 | 134 | 181 | 140 | 149 | 125 |
| ITPR1 | 26 | 24 | 19 | 15 | 18 | 16 | 20 | 19 | 24 | 23 | 15 | 13 |
| ITSN2 | 31 | 32 | 39 | 37 | 41 | 45 | 32 | 38 | 39 | 41 | 40 | 35 |
| CLCF1 | 29 | 16 | 20 | 38 | 22 | 23 | 26 | 18 | 19 | 25 | 27 | 25 |
| CRLF1 | 35 | 34 | 24 | 30 | 32 | 29 | 37 | 36 | 27 | 26 | 27 | 30 |
| IGJ | 90 | 147 | 158 | 229 | 99 | 173 | 66 | 90 | 81 | 132 | 100 | 168 |
| NFKBIZ | 506 | 1083 | 523 | 986 | 444 | 922 | 414 | 795 | 473 | 795 | 479 | 794 |
| DLC1 | 14 | 16 | 14 | 17 | 14 | 16 | 13 | 18 | 13 | 20 | 15 | 15 |
| GBP5 | 90 | 217 | 115 | 339 | 172 | 453 | 112 | 415 | 107 | 220 | 172 | 559 |
| PEG10 | 17 | 21 | 24 | 29 | 18 | 19 | 17 | 20 | 19 | 20 | 14 | 15 |
| HOMER2 | 18 | 30 | 14 | 25 | 28 | 41 | 23 | 21 | 19 | 28 | 20 | 24 |
| ZBTB8 | 15 | 17 | 18 | 16 | 14 | 14 | 13 | 12 | 16 | 12 | 14 | 12 |
| MOBKL2C | 79 | 115 | 94 | 185 | 129 | 150 | 85 | 130 | 101 | 116 | 138 | 97 |
| EDG3 | 15 | 24 | 15 | 29 | 21 | 33 | 21 | 23 | 19 | 23 | 22 | 26 |
| MELK | 21 | 18 | 20 | 24 | 19 | 19 | 21 | 22 | 16 | 26 | 16 | 19 |
| PHC3 | 32 | 30 | 24 | 27 | 32 | 38 | 27 | 29 | 25 | 28 | 35 | 34 |
| TTC3 | 120 | 79 | 72 | 37 | 89 | 98 | 86 | 50 | 74 | 46 | 115 | 90 |
| KLK1 | 50 | 67 | 41 | 68 | 59 | 59 | 64 | 49 | 68 | 44 | 60 | 52 |
| KCNV2 | 13 | 26 | 15 | 49 | 22 | 24 | 35 | 23 | 29 | 21 | 38 | 25 |
| GBP1 | 369 | 550 | 803 | 1210 | 606 | 848 | 317 | 956 | 426 | 1070 | 532 | 1122 |

TABLE 8

| | Atopic status | | | | | | | | | | | |
|---|---|---|---|---|---|---|---|---|---|---|---|---|
| | + | + | + | + | + | + | − | − | − | − | − | − |
| | Stimulus | | | | | | | | | | | |
| | C | HDM | C | HDM | C | HDM | C | HDM | C | HDM | C | HDM |
| CAMK2D | 71 | 68 | 44 | 50 | 47 | 46 | 83 | 62 | 47 | 62 | 35 | 47 |
| CDH1 | 16 | 40 | 14 | 28 | 13 | 17 | 12 | 19 | 15 | 17 | 12 | 19 |
| SLC37A3 | 21 | 35 | 16 | 31 | 17 | 29 | 19 | 31 | 17 | 26 | 17 | 27 |
| PALM2-AKAP2 | 254 | 529 | 247 | 582 | 285 | 552 | 379 | 490 | 255 | 437 | 240 | 504 |
| NSMCE1 | 459 | 545 | 443 | 549 | 399 | 442 | 441 | 447 | 434 | 436 | 407 | 396 |
| TSPAN13 | 105 | 132 | 139 | 160 | 125 | 151 | 135 | 156 | 102 | 120 | 133 | 146 |
| SYTL3 | 20 | 20 | 16 | 18 | 20 | 17 | 21 | 21 | 25 | 17 | 21 | 19 |
| SFRS8 | 93 | 51 | 66 | 66 | 116 | 109 | 90 | 77 | 119 | 49 | 126 | 114 |
| FIP1L1 | 78 | 99 | 84 | 159 | 147 | 130 | 64 | 121 | 136 | 88 | 111 | 148 |
| MAML3 | 28 | 16 | 23 | 18 | 18 | 12 | 29 | 17 | 22 | 20 | 11 | 13 |
| TRIM4 | 51 | 50 | 47 | 45 | 46 | 38 | 64 | 41 | 43 | 36 | 42 | 36 |
| SIAH1 | 199 | 138 | 149 | 116 | 131 | 91 | 159 | 112 | 184 | 116 | 143 | 113 |
| ITPR1 | 21 | 14 | 18 | 21 | 15 | 14 | 26 | 16 | 21 | 21 | 16 | 16 |
| ITSN2 | 29 | 35 | 48 | 37 | 39 | 33 | 33 | 33 | 42 | 33 | 29 | 37 |
| CLCF1 | 20 | 17 | 20 | 20 | 23 | 23 | 21 | 25 | 18 | 20 | 25 | 26 |
| CRLF1 | 32 | 30 | 36 | 37 | 33 | 27 | 32 | 31 | 37 | 33 | 41 | 27 |
| CLIC5 | 27 | 28 | 33 | 27 | 29 | 36 | 28 | 32 | 30 | 46 | 33 | 30 |
| IGJ | 87 | 185 | 88 | 167 | 92 | 154 | 59 | 146 | 51 | 100 | 85 | 173 |
| NFKBIZ | 510 | 678 | 339 | 696 | 312 | 543 | 435 | 621 | 412 | 689 | 380 | 612 |
| DLC1 | 13 | 20 | 17 | 17 | 13 | 17 | 13 | 17 | 15 | 18 | 15 | 17 |
| GBP5 | 81 | 681 | 85 | 698 | 202 | 936 | 88 | 964 | 81 | 741 | 176 | 1102 |
| PEG10 | 18 | 19 | 24 | 28 | 20 | 17 | 20 | 17 | 17 | 19 | 15 | 14 |
| HOMER2 | 18 | 24 | 21 | 31 | 28 | 45 | 28 | 33 | 21 | 36 | 24 | 24 |
| ZBTB8 | 19 | 25 | 22 | 32 | 17 | 19 | 21 | 25 | 16 | 18 | 12 | 17 |
| MOBKL2C | 75 | 112 | 82 | 160 | 120 | 140 | 78 | 113 | 86 | 131 | 56 | 87 |
| EDG3 | 14 | 20 | 14 | 17 | 19 | 24 | 16 | 17 | 23 | 19 | 21 | 17 |
| MELK | 37 | 102 | 44 | 138 | 51 | 92 | 47 | 79 | 43 | 71 | 41 | 72 |
| PHC3 | 25 | 32 | 19 | 35 | 31 | 37 | 36 | 28 | 31 | 21 | 29 | 30 |
| TTC3 | 77 | 124 | 67 | 81 | 135 | 100 | 108 | 75 | 155 | 61 | 127 | 92 |
| SEL1 | 32 | 37 | 35 | 41 | 36 | 48 | 30 | 74 | 27 | 37 | 16 | 55 |
| IL1R2 | 82 | 259 | 153 | 403 | 163 | 239 | 100 | 677 | 103 | 473 | 90 | 273 |
| IFI44L | 39 | 93 | 147 | 231 | 80 | 308 | 16 | 97 | 24 | 87 | 26 | 201 |
| LIX1L | 67 | 51 | 72 | 47 | 71 | 60 | 51 | 72 | 49 | 63 | 35 | 71 |

### Example 2

### qRT-PCR Validation of Results in Example 1

Real-time quantitative PCR was performed to measure expression levels of the index gene IL-4 in RNA extracts from cell pellets from the individual samples used to generate the pools for the kinetic experiment in Example 1, using ABI Prism 7900HT Sequence Detection System. The rationale was the necessity to confirm the “Th2 status” of each sample, using the criterion of positive expression of the gene, which is the essential growth factor for all Th2 cells.

Standard PCR premixes were prepared using QuantiTect SYBRGreen PCR Master Mix (QIAGEN), containing 2.5 mM MgCl₂ (final concentration). SYBR®Green binds to all double-stranded DNA, so no probe was needed. Primers were designed in-house (Sequences are listed below) and used at a concentration of 0.3 μM. Alternatively QuantiTect Primer Assays (QIAGEN Catalogue Nos QT00026201 (CAMK2D), QT00047593 (NSMCE1), QT00038892(TSPAN13), and QT00062755 (STYL3)) were used. Standard conditions were used, except that 15 minutes instead of 10 minutes was used for HotStar Taq polymerase activation. In addition, a dissociation step was included and melt curve analysis performed to confirm amplification of a single product. Amplified products were or will be sequenced to confirm specific amplification of the target of interest.

The in-house primers used for the PCR were:

| | |
|---|---|
| IL-4 Forward Primer: | |
| AAC AGC CTC ACA GAG CAG AAG ACT | SEQ ID NO. 1 |
| IL-4 Reverse Primer: | |
| CAG CGA GTG TCC TTC TCA TGG T | SEQ ID NO. 2 |

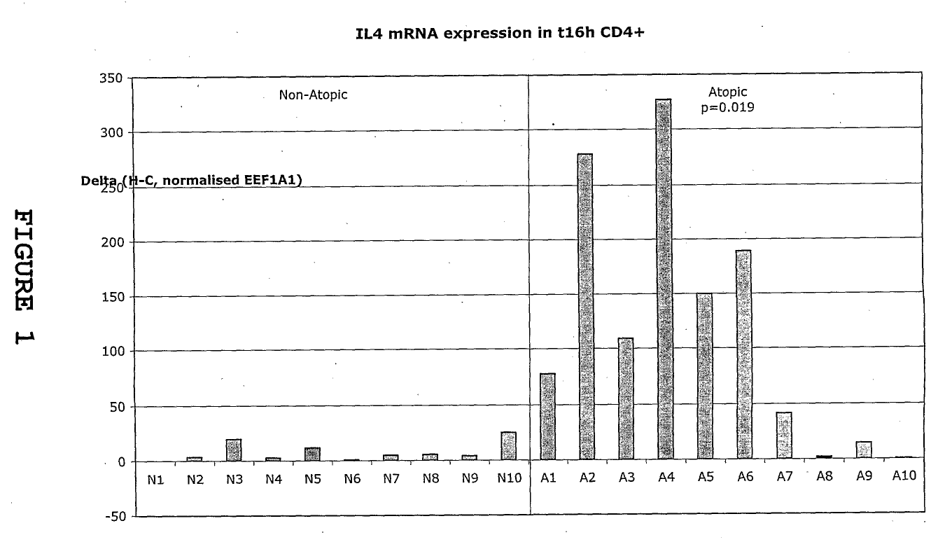

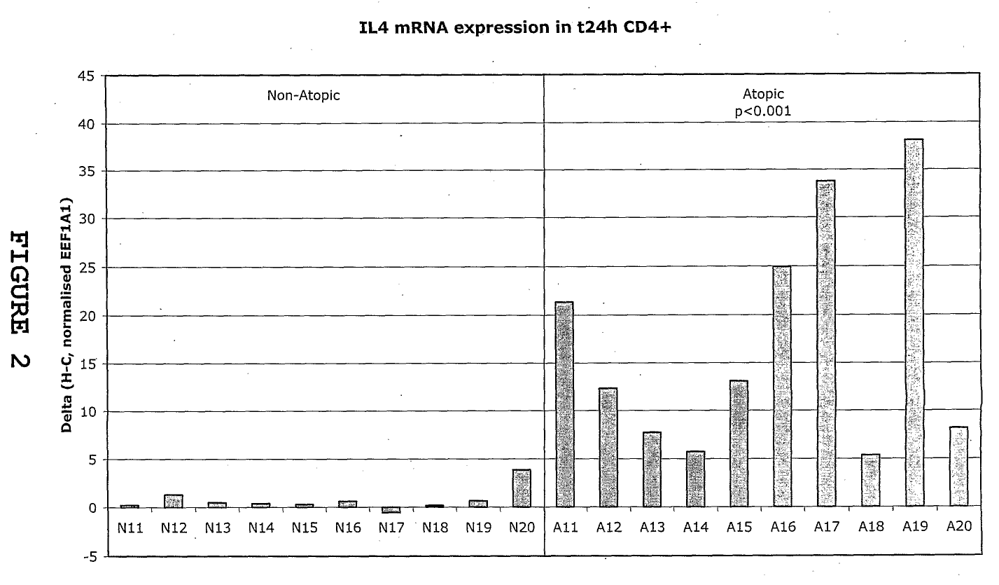

The data were normalised to the EEF1A1 housekeeping gene. Expression of IL-4 is illustrated in FIGS. 1 and 2. The results are shown as delta values (difference between unstimulated and HDM-stimulated cultures) for non-allergic (N) and allergic (A) individuals.

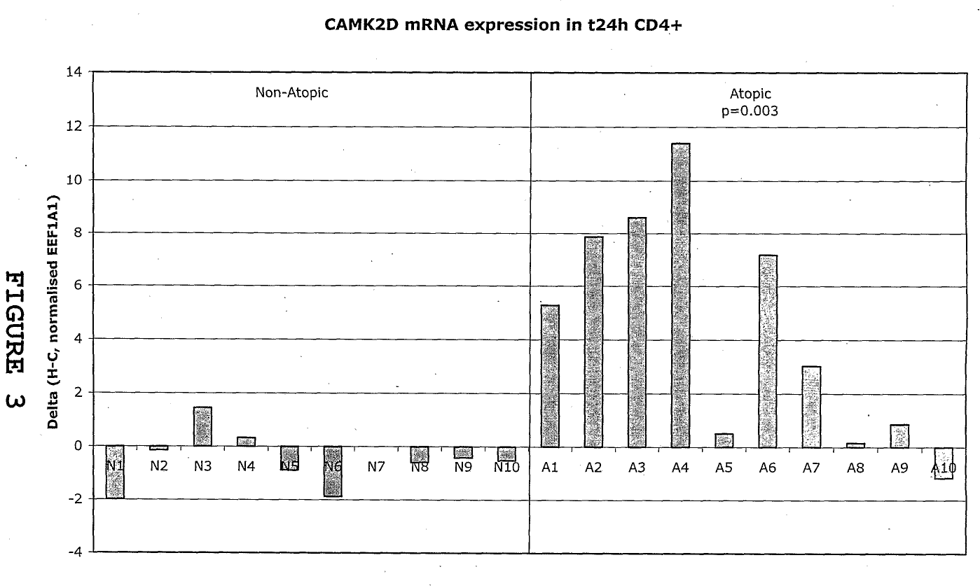

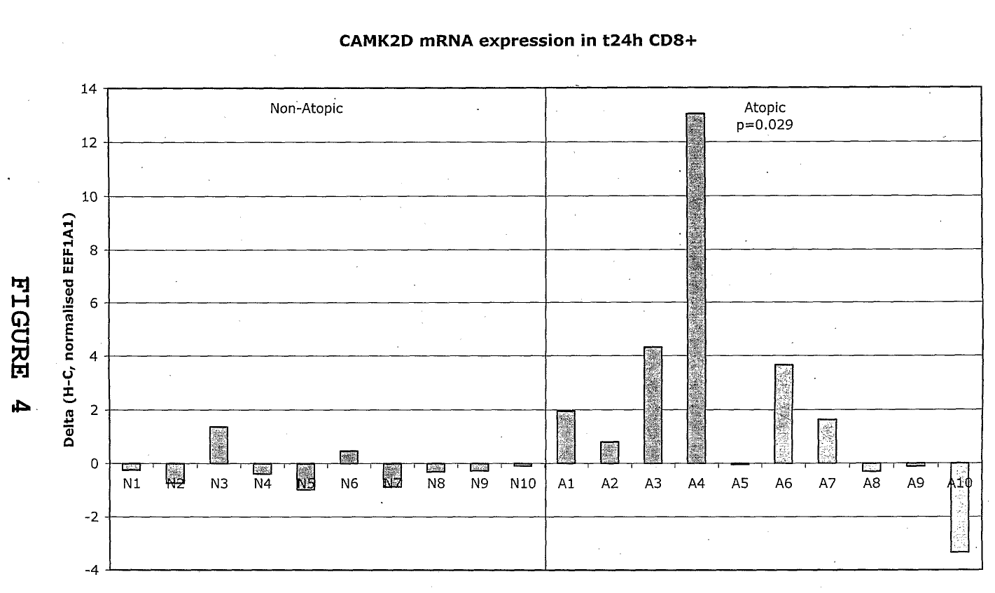

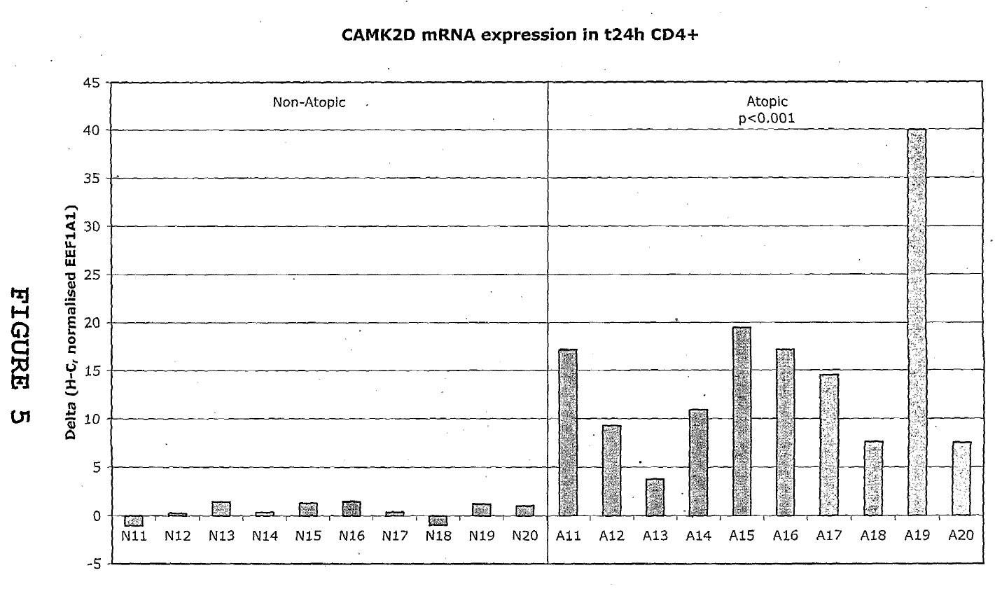

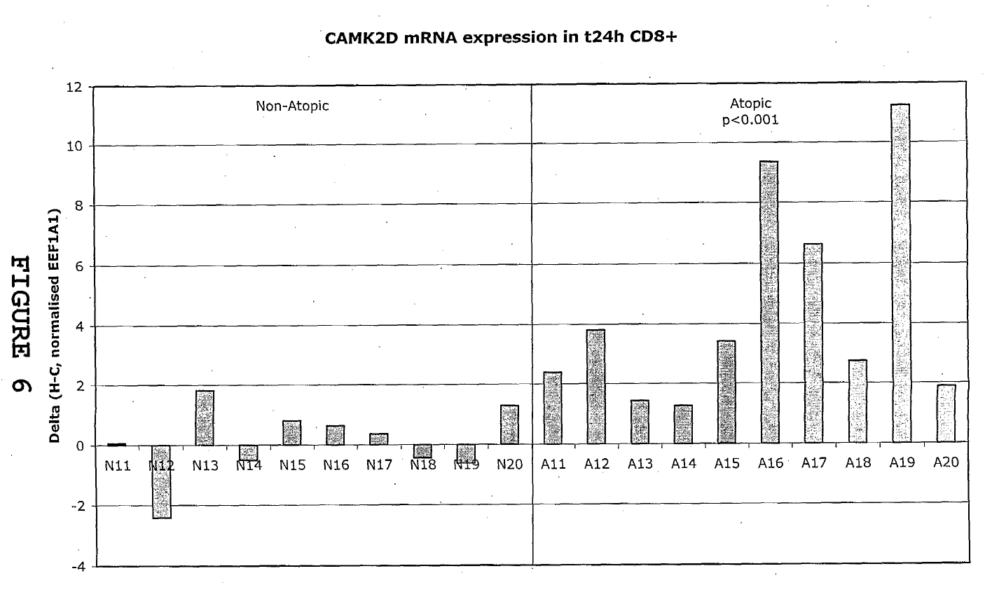

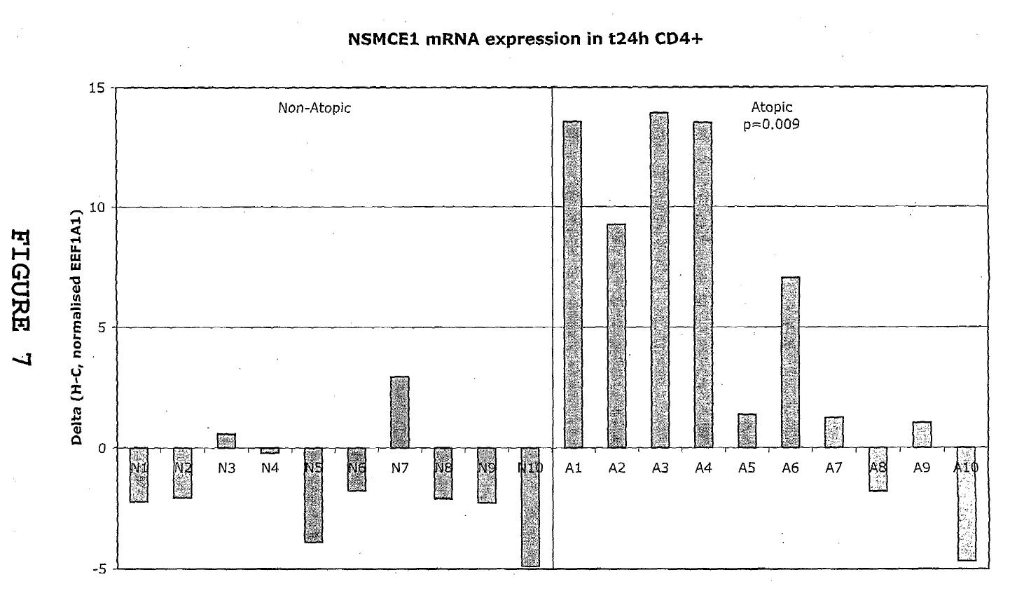

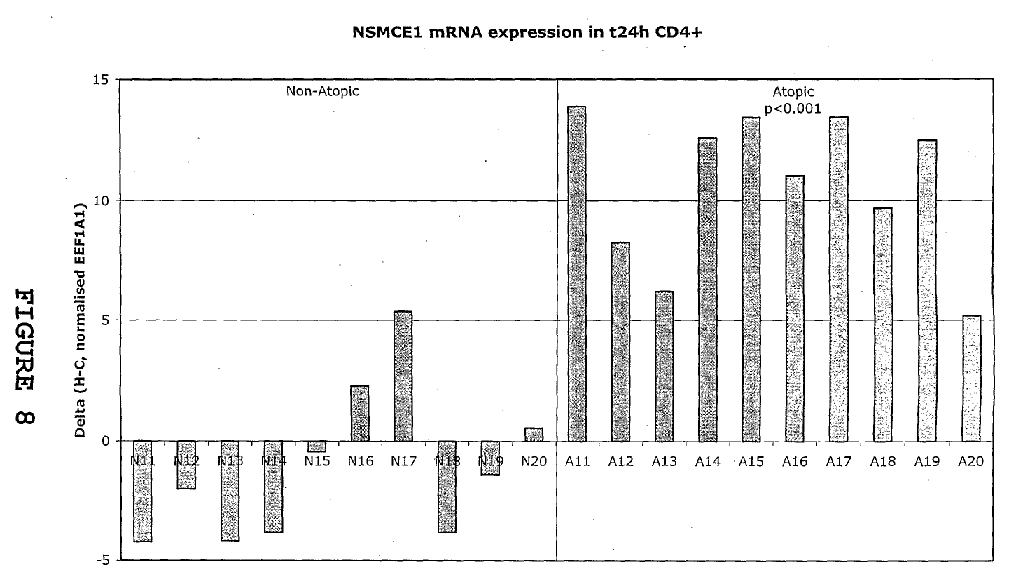

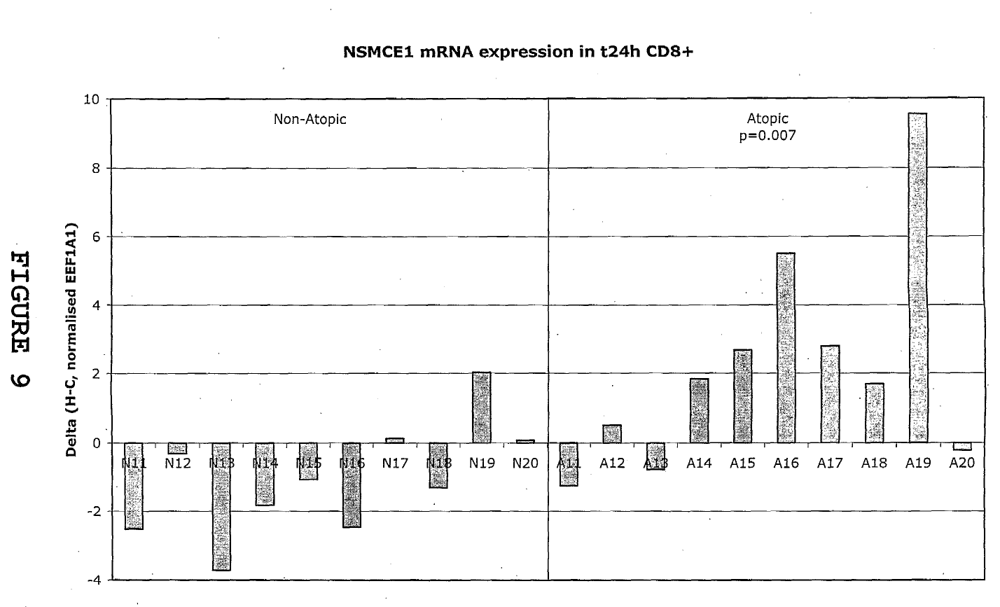

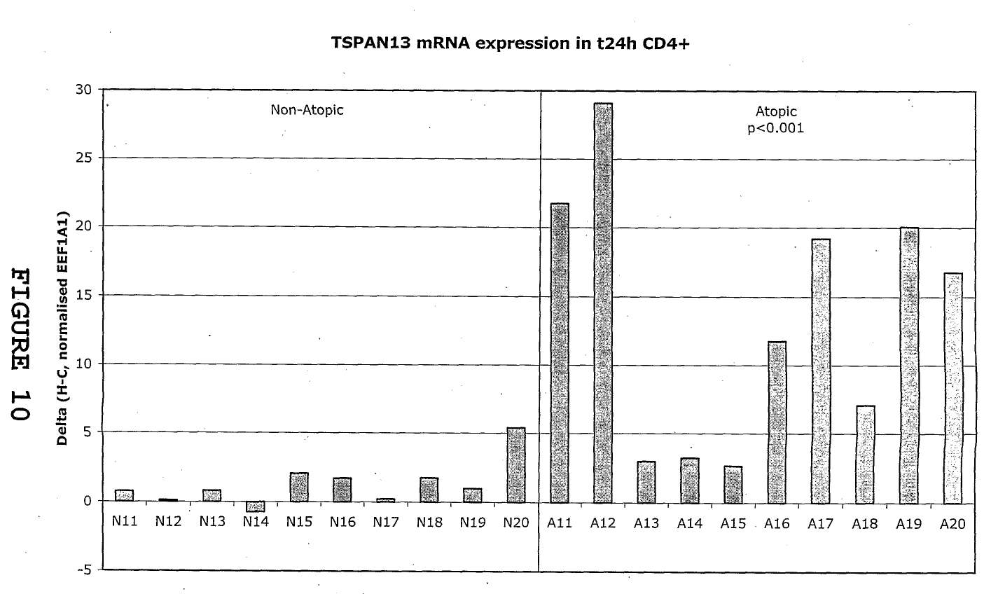

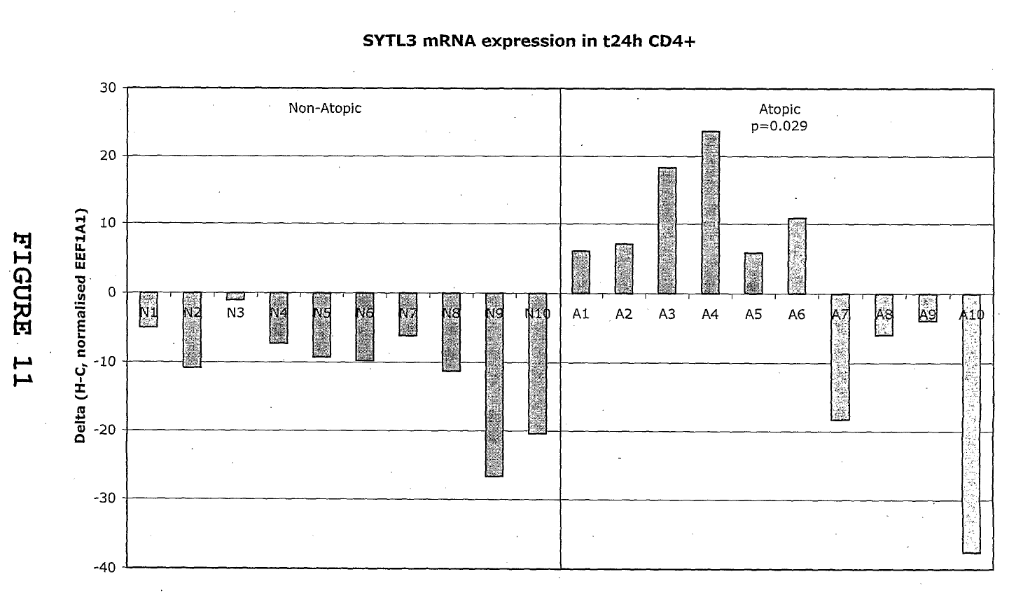

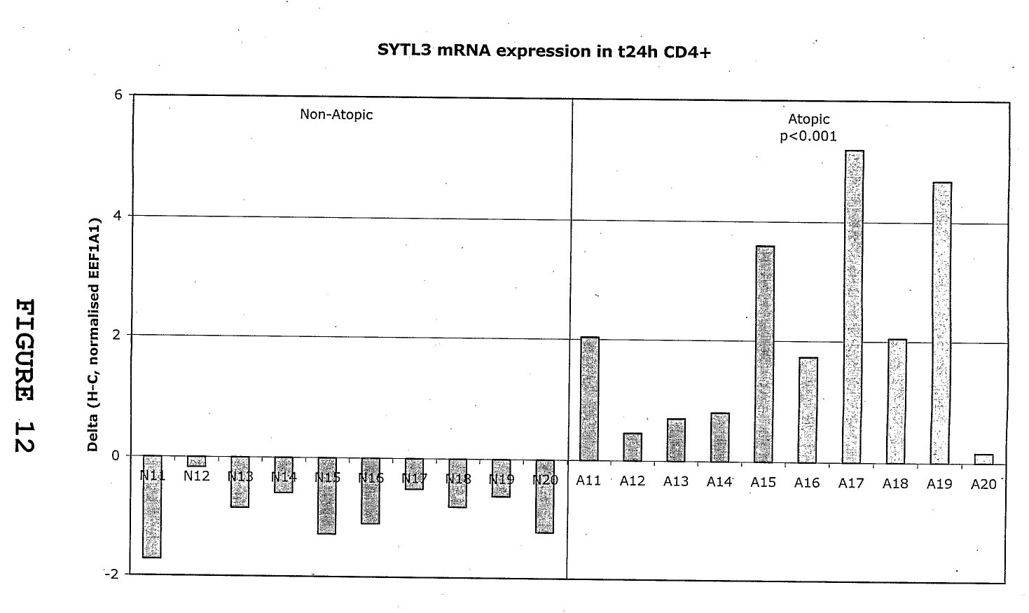

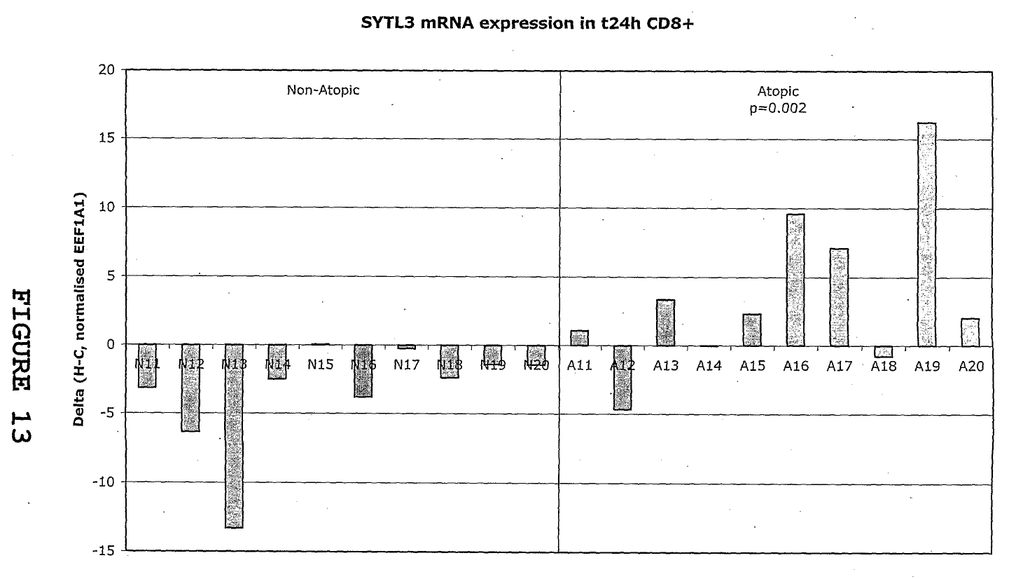

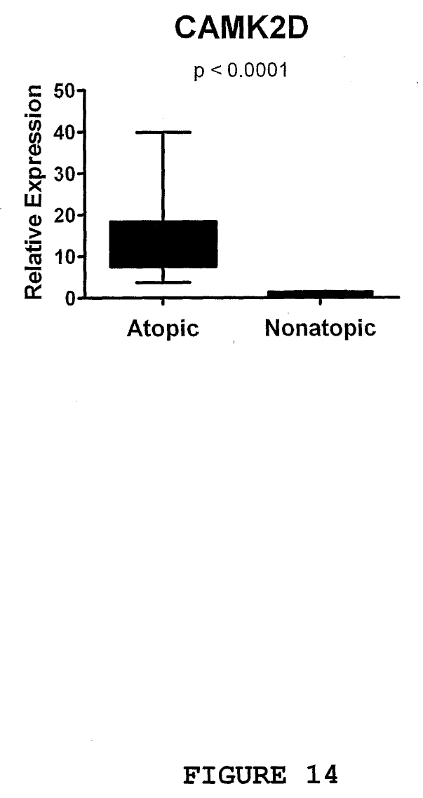

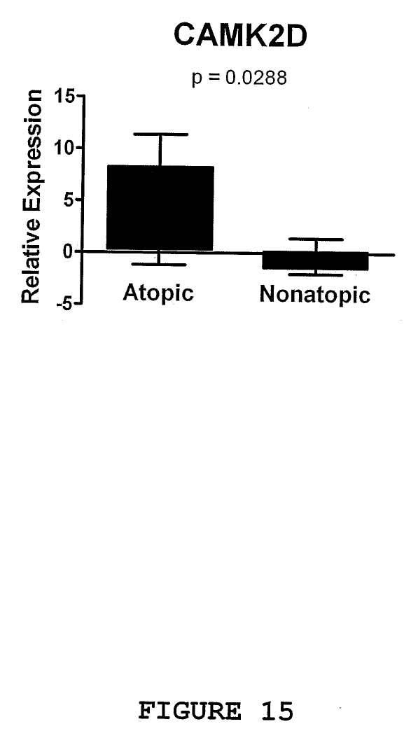

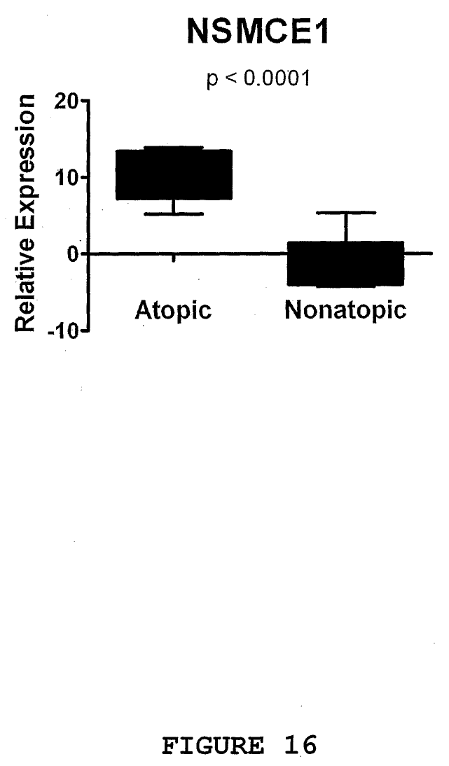

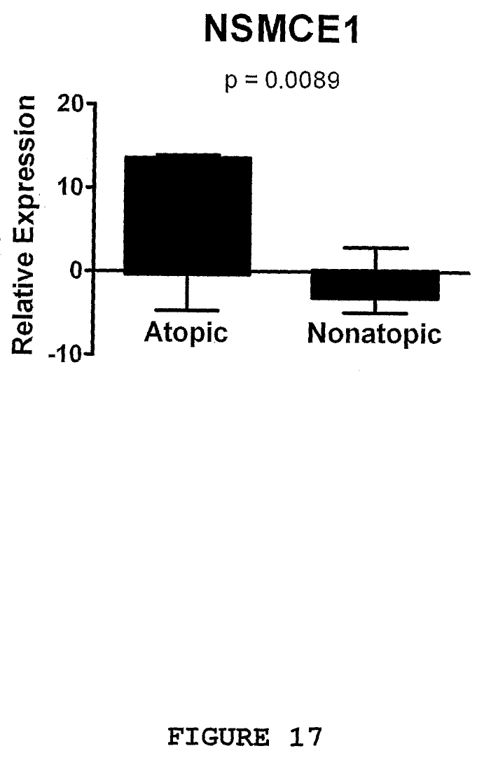

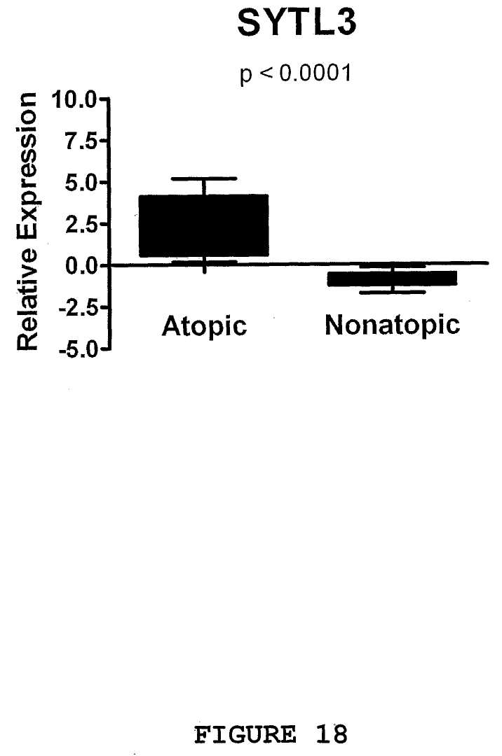

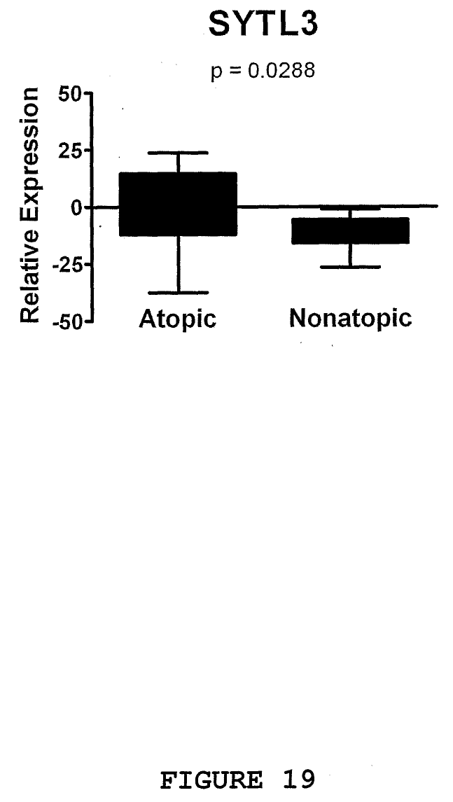

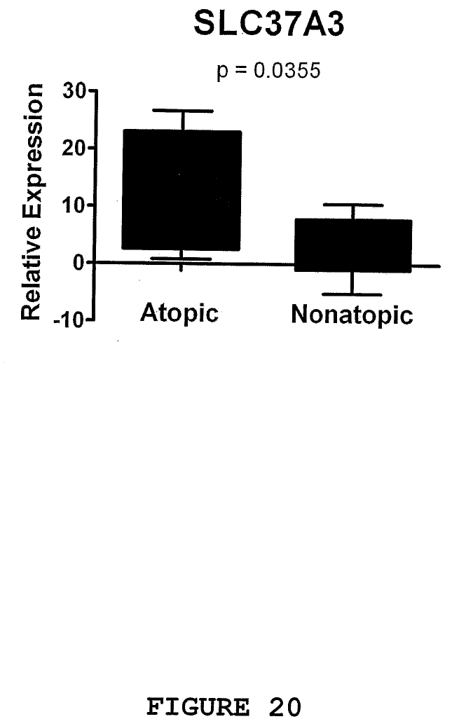

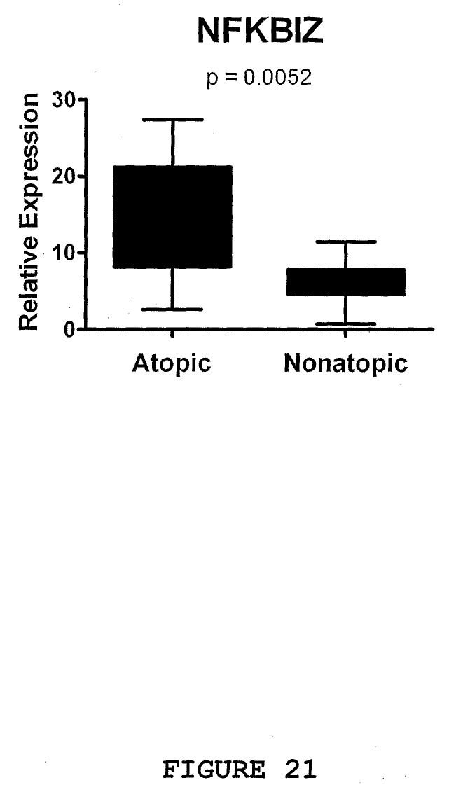

Validation experiments were performed for CAMK2D, NSMCE1, TSPAN13 and SYTL3 by quantitative RT-PCR. RNA from the individual samples employed to generate the pools used for microarray analysis at the 24 hr time point in purified CD4 or CD8 T cells was converted to cDNA, and then quantitative RT-PCR was performed using SYBR®Green and QuantiTect Primer Assays (QIAGEN). Data were normalised to the EEF1A1 housekeeping gene. The results shown in FIGS. 3 to 21 demonstrate that CAMK2D, NSMCE1, TSPAN13 and SYTL3 are significantly upregulated to HDM in allergic subjects compared to non-allergic subjects, while IL1F9, GBP1, SEL1, IL1R2, IFI44L, and LIX1L were upregulated to a greater extent in non-allergic subjects compared to allergic subjects.

### Example 3

### Confirmation of the Role Genes in the Overall TH2 Network Employing Alternative Statistical Methodology

Cellular processes are orchestrated by complex networks of interacting proteins (derived from genes), and different combinations of proteins are networked (or interconnected) together in sub networks called modules to perform specific tasks. The Th2 gene network responsible for allergic disease in an archetypal example. Recent developments in gene network theory (Barabasi and Oltvai (2004) Nat Rev Genet. 5: 101-13) and statistical methodologies (Zhang and Horvath (2005) Stat Appl Genet Mol. Biol. 4) can now be applied to microarray data allowing a network-level interpretation of microarray experiments. We have taken advantage of these developments to further validate the novel “Th2-associated genes” covered in this patent. In doing so we have generated larger data sets describing house dust mite responses in atopics and non-atopics obtained from additional microarray experiments performed on similar patient groups.

To identify functional modules in T cell responses to allergens in this data set, genes which were significantly modulated (p. value <0.05 from Bayesian T-test after false discovery rate correction for multiple testing (1239 genes in total; Smyth G K. (2004), Stat Appl Genet Mol. Biol. 3) in response to stimulation of peripheral blood T cells with house dust mite allergen were analysed further employing weighted gene co-expression network analysis methods (Zhang and Horvath (2005) Stat Appl Genet Mol. Biol. 4). Briefly, the absolute Pearson correlation was calculated between each pair of the 1239 genes, and the resulting data matrix was transformed into a measure of the gene-gene pairwise connection strengths (shown as “Connectivity” on figure). Average linkage hierarchical clustering was then used to identify modules of genes with high interconnectivity, and the resulting weighted gene co-expression network consisted of 1239 genes which were divided by the clustering algorithm (Carlson et al. (2006) BMC Genomics. 7: 40) into 16 separate functional modules.

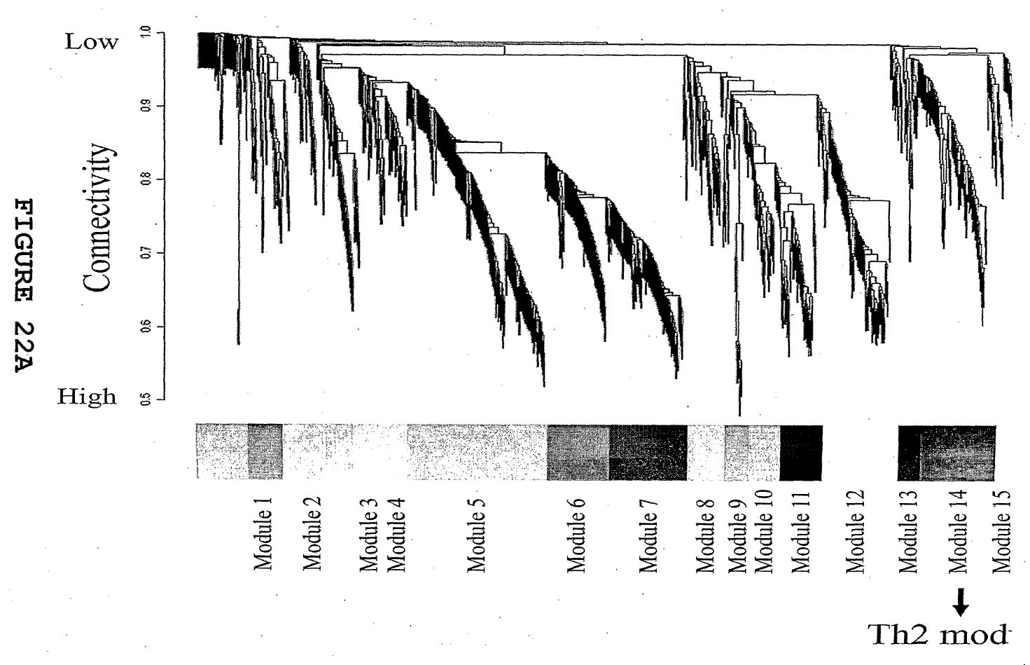

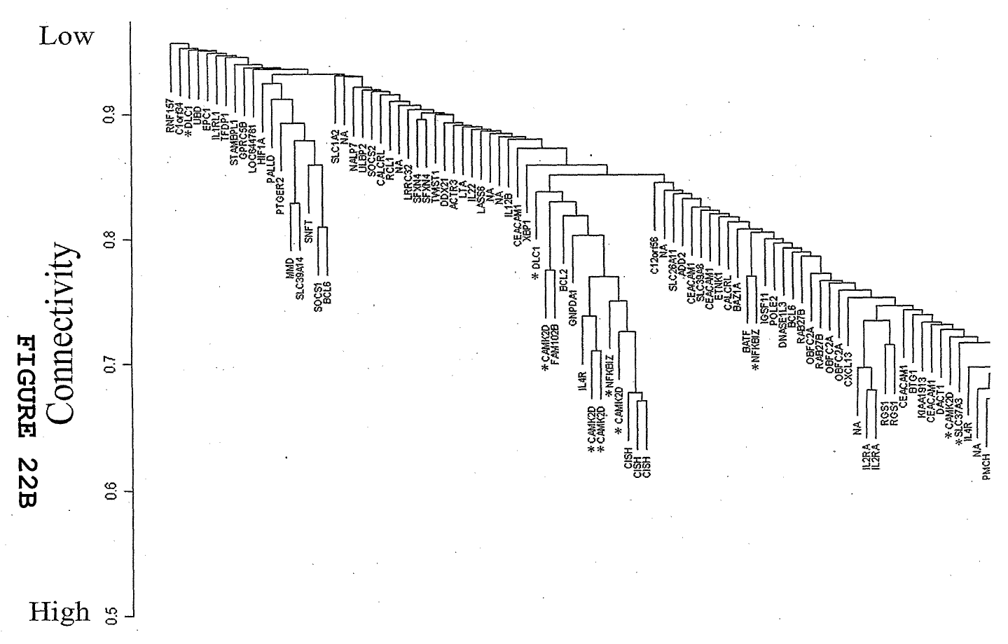

The co-expression network comprising the 16 functional modules is illustrated in its entirety in FIG. 22A, where the tree-like dendrogram connects genes together that have high interconnectivity (correlated expression levels), revealing separate branch-like structures of highly connected genes or network modules. Note that smaller values on the vertical axis indicate higher connectivity.

Closer inspection of the co-expression network revealed that the principal genes mediating Th2-driven allergic inflammation (IL-4, IL-4R, IL-5, IL-9, IL-13) formed a “Th2 effector” module (module 14 in FIG. 22A) with 104 other genes, and this subset of the network is expanded in FIG. 22B. The genes which are the subject of this patent are marked with “*” in FIG. 22B and comprise: CAMK2D, CDH1, DLC1, NFKBIZ, NSMCE1, SLC37A3.

1. A method for predicting the development of an allergic disorder in a mammal comprising the steps of: (a) contacting a cell of the mammal with an allergen; (b) contacting a cell of a non-allergic mammal with the same allergen used in step (a); (c) obtaining a sample of nucleic acid isolated from the cells in steps (a) and (b), wherein the nucleic acid is RNA or a cDNA copy of RNA; (d) determine the gene expression pattern of a panel of specific sequences comprising CAMK2D and CDH1 within each nucleic acid pool described in (c) that have been predetermined to either increase or decrease in response to allergy, where the gene expression pattern comprises the relative level of mRNA or cDNA abundance for the panel of specific sequences; and (e) compare the expression patterns in step (d), wherein the difference in the levels of expression is predictive of whether the mammal in step (a) will develop allergy.

2. A method for diagnosing an allergic disorder in a mammal comprising the steps of: (a) contacting a cell of the mammal with an allergen; (b) contacting a cell of a non-allergic mammal with the same allergen used in step (a); (c) obtaining a sample of nucleic acid isolated from the cells in steps (a) and (b), wherein the nucleic acid is RNA or a cDNA copy of RNA; (d) determine the gene expression pattern of a panel of specific sequences comprising CAMK2D and CDH1 within each nucleic acid pool described in (c) have been predetermined to either increase or decrease in response to allergy, where the gene expression pattern comprises the relative level of mRNA or cDNA abundance for the panel of specific sequences; and (e) compare the expression patterns in step (d), wherein the difference in the levels of expression is diagnostic that the mammal in step (a) is allergic.

3. A method according to claim 1, wherein the panel of specific sequences further comprises one or more of SLC37A3, PALM2-AKAP2, NSMCE1, TSPAN13, SYTL3, SFRS8, FIP1L1, MAML3, TRIM4, SIAH1, ITPR1, ITSN2, CLCF1, CRLF1, CLIC5, IGJ, NFKBIZ, DLC1, GBP5, PEG10, HOMER2, ZBTB8, MOBKL2C, EDG3, MELK, PHC3, TTC3, KLK1, KCNV2, IL1F9, GBP1, SEL1, IL1R2, IFI44L or LIX1L.

4. A method according to claim 1, wherein the cell is a peripheral blood mononuclear cell (PBMC).

5. A method for preventing or treating an allergic disorder in a mammal comprising the steps of:
(a) obtaining a pool of nucleic acid molecules isolated from the mammal's organ, tissue or cell, wherein the nucleic acid is RNA or a cDNA copy of RNA;
(b) determining the gene expression pattern of a panel of specific sequences within the pool of nucleic acid molecules described in (a) that have been predetermined to either increase or decrease in response to allergy, where the gene expression pattern comprises the relative level of mRNA or cDNA abundance for the panel of specific sequences and wherein said panel includes CAMK2D and CDH1;
(c) identify a gene expression pattern for one or more of the panel of specific sequences which is different when compared with the predetermined level of expression; and
(d) administering an agent capable of bringing the gene expression pattern to the predetermined level of expression.

6. A method of selecting an agent for the treatment of a mammal having an allergic disorder, comprising:
(a) contacting a cell of an allergic mammal with a test agent;
(b) contacting a cell of a non-allergic mammal with the same test agent used in step (a);
(c) obtaining a sample of nucleic acid isolated from the cells in steps (a) and (b), wherein the nucleic acid is RNA or a cDNA copy of RNA;
(d) determine the gene expression pattern of a panel of specific sequences comprising CAMK2D and CDH1 within each nucleic acid pool described in (c) that have been predetermined to either increase or decrease in response to allergy, where the gene expression pattern comprises the relative level of mRNA or cDNA abundance for the panel of specific sequences; and
(e) compare the expression patterns in step (d), and if the levels of expression of said panel are similar then the test agent is useful in the treatment of a mammal with an allergy.

7. A method of selecting a prophylactic agent for a mammal in which an allergic disorder is to be prevented, comprising:
(a) contacting a cell of suspected allergic mammal with a test agent;
(b) contacting a cell of a non-allergic mammal with the same test agent used in step (a);
(c) obtaining a sample of nucleic acid isolated from the cells in steps (a) and (b), wherein the nucleic acid is RNA or a cDNA copy of RNA;
(d) determine the gene expression pattern of a panel of specific sequences comprising CAMK2D and CDH1 within each nucleic acid pool described in (c) that have been predetermined to either increase or decrease in response to allergy, where the gene expression pattern comprises the relative level of mRNA or cDNA abundance for the panel of specific sequences; and
(e) compare the expression patterns in step (d), and if the levels of expression of said panel are similar then the test agent is useful as a prophylactic agent in the prevention of an allergy in the mammal.

8. A control agent capable of modulating the expression of a gene associated with an allergic disorder:
(a) contacting a cell of an allergic mammal with a test agent;
(b) contacting a cell of a non-allergic mammal with the same test agent used in step (a);
(c) obtaining a sample of nucleic acid isolated from the cells in steps (a) and (b), wherein the nucleic acid is RNA or a cDNA copy of RNA;
(d) determine the gene expression pattern of a panel of specific sequences comprising CAMK2D and CDH1 within each nucleic acid pool described in (c) that have been predetermined to either increase or decrease in response to allergy, where the gene expression pattern comprises the relative level of mRNA or cDNA abundance for the panel of specific sequences; and
(e) compare the expression patterns in step (d), and if the levels of expression of said panel are different in the presence of the test agent this indicates that the agent is capable of modulating the expression of CAMK2D and CDH1.

9. A method of monitoring a mammal during therapy for an allergic disorder, comprising:
(a) contacting a cell of the mammal before therapy with an allergen;
(b) contacting a cell of the mammal under therapy with the same allergen used in step (a);
(c) contacting a cell of a non-allergic mammal with the same allergen used in step (a);
(d) obtaining a sample of nucleic acid isolated from the cells in steps (a), (b) and (c), wherein the nucleic acid is RNA or a cDNA copy of RNA;
(e) determine the gene expression pattern of a panel of specific sequences comprising CAMK2D and CDH1 within each nucleic acid pool described in (d) that have been predetermined to either increase or decrease in response to allergy, where the gene expression pattern comprises the relative level of mRNA or cDNA abundance for the panel of specific sequences; and
(f) compare the expression patterns in step (e) and determine whether the level of expression has changed during therapy,
wherein a change in the level of expression during therapy is an indication of the progress of the therapy.

10. A method of determining the potential responsiveness of an animal suffering from an allergic disorder to treatment for the allergic disorder, comprising:
(a) contacting a cell of an allergic mammal with an allergen;
(b) contacting a cell of a non-allergic mammal with the same allergen used in step (a);
(c) obtaining a sample of nucleic acid isolated from the cells in steps (a) and (b), wherein the nucleic acid is RNA or a cDNA copy of RNA;
(d) determine the gene expression pattern of a panel of specific sequences comprising CAMK2D and CDH1 within each nucleic acid pool described in (c) that have been predetermined to either increase or decrease in response to allergy, where the gene expression pattern comprises the relative level of mRNA or cDNA abundance for the panel of specific sequences; and
(e) compare the expression patterns in step (d), wherein a difference in the levels of expression is indicative of the potential responsiveness of the animal to the therapy.

11. A method of predicting the risk of an animal suffering from an allergic disorder progressing to a more severe and/or persistent form of the allergic disorder, comprising:
(a) contacting a cell of an allergic mammal with an allergen;
(b) contacting a cell of a non-allergic mammal with the same allergen used in step (a);
(c) obtaining a sample of nucleic acid isolated from the cells in steps (a) and (b), wherein the nucleic acid is RNA or a cDNA copy of RNA;
(d) determine the gene expression pattern of a panel of specific sequences comprising CAMK2D and CDH1 within each nucleic acid pool described in (c) that have been predetermined to either increase or decrease in response to allergy, where the gene expression pattern comprises the relative level of mRNA or cDNA abundance for the panel of specific sequences; and
(e) compare the expression patterns in step (d), wherein any difference in the level of expression between the allergic mammal and non-allergic mammal is predictive of the risk of the allergic mammal developing a more severe and/or persistent form of the allergic disorder.

12. A method of determining the immunological phenotype of an allergic disorder in an animal, comprising:
(a) contacting a cell of an allergic mammal with an allergen;
(b) contacting a cell of a non-allergic mammal with the same allergen used in step (a);
(c) obtaining a sample of nucleic acid isolated from the cells in steps (a) and (b), wherein the nucleic acid is RNA or a cDNA copy of RNA;
(d) determine the gene expression pattern of a panel of specific sequences comprising CAMK2D and CDH1 within each nucleic acid pool described in (c) that have been predetermined to either increase or decrease in response to allergy, where the gene expression pattern comprises the relative level of mRNA or cDNA abundance for the panel of specific sequences; and
(e) compare the expression patterns in step (d), wherein the level of expression is indicative of the immunological phenotype of the animal.

13. An isolated molecule comprising one or more of:
a) the sequence of a nucleic acid selected from the group consisting of CAMK2D, CDH1, SLC37A3, PALM2-AKAP2, NSMCE1, TSPAN13, SYTL3, SFRS8, FIP1L1, MAML3, TRIM4, SIAH1, ITPR1, ITSN2, CLCF1, CRLF1, CLIC5, IGJ, NFKBIZ, DLC1, GBP5, PEG10, HOMER2, ZBTB8, MOBKL2C, EDG3, MELK, PHC3, TTC3, KLK1, KCNV2, IL1F9, GBP1, SEL1, IL1R2, IFI44L and LIX1L, or a biologically active fragment thereof;
b) an isolated nucleic acid molecule which is the complement of a sequence of a);
c) an isolated nucleic molecule which hybridises under stringent conditions to a nucleic acid molecule of a) or b); and/or
d) an isolated polypeptide encoded by a nucleic acid molecule of a), b) or c), for use in the treatment or prevention of an allergic disorder.

14. A therapeutic or prophylactic agent, comprising one or more of:
a) an isolated nucleic acid molecule having the sequence of a nucleic acid selected from the group consisting of CAMK2D, CDH1, SLC37A3, PALM2-AKAP2, NSMCE1, TSPAN13, SYTL3, SFRS8, FIP1L1, MAML3, TRIM4, SIAH1, ITPR1, ITSN2, CLCF1, CRLF1, CLIC5, IGJ, NFKBIZ, DLC1, GBP5, PEG10, HOMER2, ZBTB8, MOBKL2C, EDG3, MELK, PHC3, TTC3, KLK1, KCNV2, IL1F9, GBP1, SEL1, IL1R2, IFI44L and LIX1L, or a biologically active fragment thereof;
b) an isolated nucleic acid molecule which is the complement of a sequence of a);
c) an isolated nucleic molecule which hybridises under stringent conditions to a nucleic acid molecule of a) or b); and/or
d) an isolated polypeptide encoded by a nucleic acid molecule of a), b) or c), together with a pharmaceutically acceptable carrier.

15. An agent according to claim 15, wherein the agent is for use in the treatment or prevention of an allergic disorder.

16. A method of treating or preventing an allergic disorder, comprising the step of administering to a mammal one or more of:
a) an isolated nucleic acid molecule having the sequence of a gene selected from the group consisting of CAMK2D, CDH1, SLC37A3, PALM2-AKAP2, NSMCE1, TSPAN13, SYTL3, SFRS8, FIP1L1, MAML3, TRIM4, SIAH1, ITPR1, ITSN2, CLCF1, CRLF1, CLIC5, IGJ, NFKBIZ, DLC1, GBP5, PEG10, HOMER2, ZBTB8, MOBKL2C, EDG3, MELK, PHC3, TTC3, KLK1, KCNV2, IL1F9, GBP1, SEL1, IL1R2, IFI44L and LIX1L, or a biologically active fragment thereof;
b) an isolated nucleic acid molecule which is the complement of a sequence of a);
c) an isolated nucleic molecule which hybridises under stringent conditions to a nucleic acid molecule of a) or b);
d) an isolated polypeptide encoded by a nucleic acid molecule of a), b) or c); and/or
e) an agent capable of modulating the expression of a molecule of a), b), and/or c), or which specifically binds a polypeptide of d).

17. A method according to claim 17, wherein the agent is antisense to the nucleic acid sequence of a gene selected from the group consisting of CAMK2D, CDH1, SLC37A3, PALM2-AKAP2, NSMCE1, TSPAN13, SYTL3, SFRS8, FIP1L1, MAML3, TRIM4, SIAH1, ITPR1, ITSN2, CLCF1, CRLF1, CLIC5, IGJ, NFKBIZ, DLC1, GBP5, PEG10, HOMER2, ZBTB8, MOBKL2C, EDG3, MELK, PHC3, TTC3, KLK1, KCNV2, IL1F9, GBP1, SEL1, IL1R2, IFI44L and LIX1L, or a biologically active fragment thereof.

18. A method according to claim 2, wherein the panel of specific sequences further comprises one or more of SLC37A3, PALM2-AKAP2, NSMCE1, TSPAN13, SYTL3, SFRS8, FIP1L1, MAML3, TRIM4, SIAH1, ITPR1, ITSN2, CLCF1, CRLF1, CLIC5, IGJ, NFKBIZ, DLC1, GBP5, PEG10, HOMER2, ZBTB8, MOBKL2C, EDG3, MELK, PHC3, TTC3, KLK1, KCNV2, IL1F9, GBP1, SEL1, IL1R2, IFI44L or LIX1L.

19. A method according claim 2, wherein the cell is a peripheral blood mononuclear cell (PBMC).
